(19) United States
(12) Patent Application Publication
(10) Pub. No.: US 2025/0012806 A1
(43) Pub. Date: Jan. 9, 2025

(54) METHODS AND SYSTEMS FOR SUPER-RESOLUTION STUDY OF THERAPIES

(71) Applicant: Toreador Therapeutics, Inc., San Francisco, CA (US)

(72) Inventors: Jeanmarie GUENOT, San Francisco, CA (US); Klaus FIEBIG, San Francisco, CA (US)

(21) Appl. No.: 18/706,534

(22) PCT Filed: Nov. 2, 2022
(86) PCT No.: PCT/US2022/048753

§ 371 Date: May 1, 2024

Related U.S. Application Data
(60) Provisional application No. 63/371,355, filed on Aug. 12, 2022.
(60) Provisional application No. 63/275,253, filed on Nov. 3, 2021.

(51) Int. Cl. G01N 33/68; G01N 33/50; G01N 33/532; G01N 33/574

(52) U.S. Cl. CPC G01N 33/6803; G01N 33/5088; G01N 33/532; G01N 33/57484; G01N 2800/50

(57) ABSTRACT

Disclosed herein are methods of generating a database of cellular features from disease-state cells and non-disease state cells using super-resolution microscopy. Also disclosed herein are methods of designing antibody-based therapies and identifying cancer targets for therapeutic intervention by using super-resolution microscopy. Methods of characterizing cell and tissue samples using super-resolution microscopy are also disclosed.

### CROSS-REFERENCE

The present application claims the benefit of U.S. Provisional Application No. 63/275,253, filed on Nov. 3, 2021, and U.S. Provisional Application No. 63/371,355, filed on Aug. 12, 2022, which are incorporated by reference in their entirety.

### SUMMARY

Disclosed herein are methods of generating a database comprising cellular features from a disease-state cell and non-disease state cell comprising imaging a sample that has been contacted by an antibody using a super-resolution microscope and generating a database comprising at least one cellular feature of a cell in the sample and determining if the at least one cellular feature is indicative of the disease-state cell or the non-disease-state cell.

In some embodiments, the at least one cellular feature comprises an image of an extracellular molecule. In some embodiments, the at least one cellular feature comprises two- or three-dimensional coordinates of an extracellular molecule. In some embodiments, the at least one cellular feature comprises intensities for an extracellular molecule. In some embodiments, the at least one cellular feature comprises all or part of a schematic or topical representation of an extracellular molecule on a cell or tissue. In some embodiments, the at least one cellular feature comprises proximity between two or more extracellular molecules. In some embodiments, the two or more extracellular molecules are of the same target or different targets. In some embodiments, the at least one cellular feature comprises interactions of at least two extracellular molecules. In some embodiments, the at least one cellular feature comprises a distance or distances between two or more extracellular molecules. In some embodiments, the at least one cellular feature comprises an individual extracellular molecule or pairs of extracellular molecules. In some embodiments, the individual extracellular molecule or pairs of extracellular molecules are adjacent or in contact with each other. In some embodiments, the individual extracellular molecule or pairs of extracellular molecules are within a distance of about 1 nanometer of each other. In some embodiments, the individual extracellular molecule or pairs of extracellular molecules are with a distance of about 1 nanometer to about 100 nanometers of each other. In some embodiments, the at least one cellular feature comprises individual, pairs, or triplets of extracellular molecules. In some embodiments, the individual, pairs, or triplets of extracellular molecules are adjacent or in contact with each other. In some embodiments, the individual, pairs, or triplets of extracellular molecules are within a distance of about 1 nanometer of each other. In some embodiments, the individual, pairs, or triplets of extracellular molecules are within a distance of about 1 nanometer to about 100 nanometers of each other. In some embodiments, the at least one cellular feature comprises pairs, triplets, quadruplets, quintuplets, or higher order clusters of extracellular molecules. In some embodiments, the pairs, triplets, quadruplets, quintuplets, or higher order clusters of extracellular molecules are adjacent or in contact with each other. In some embodiments, the pairs, triplets, quadruplets, quintuplets or higher order clusters of extracellular molecules comprise the same or different extracellular molecules. In some embodiments, the pairs, triplets, quadruplets, quintuplets, or higher order clusters or extracellular molecules are within a distance of about 1 nanometer of each other. In some embodiments, the pairs, triplets, quadruplets, quintuplets, or higher order clusters of extracellular molecules are within a distance of about 1 nanometer to about 100 nanometers of each other. In some embodiments, the at least one cellular feature comprises a geometric relationship between two or more extracellular molecules. In some embodiments, the at least one cellular feature comprises movement of at least one extracellular molecule. In some embodiments, the movement is internalization or movement to an extracellular surface. In some embodiments, the at least one cellular feature comprises internalization of at least one extracellular molecule. In some embodiments, the at least one cellular feature comprises a change in two- or three-dimensional coordinates of an extracellular molecule.

In some embodiments, the at least one cellular feature comprises a change in clustering of an extracellular molecule. In some embodiments, the at least one cellular feature comprises an oligomerization state of an extracellular molecule. In some embodiments, the at least one cellular feature comprises a clustering state of an extracellular molecule. In some embodiments, the at least one cellular feature comprises a clustering state of an extracellular molecule. In some embodiments, the extracellular molecule is an extracellular molecule listed in Table 1. In some embodiments, the at least one cellular feature is present on a cell in tissue. In some embodiments, the at least one cellular feature is present on a cell in cancer or on normal tissue. In some embodiments, the at least one cellular feature is present on a normal cell, on a cancer cell or both normal and cancer cells. In some embodiments, the at least one cellular feature is present on a normal cell, a cancer cell or a cell in the tumor microenvironment. In some embodiments, the at least one cellular feature is present on a cell in the tumor microenvironment. In some embodiments, the cell in the tumor microenvironment is selected from a B cell, a T cell, a Natural Killer (NK) cell, a myeloid cell, a fibroblast, and a pericyte. In some embodiments, the cell in the tumor microenvironment is selected from a stromal cell, an epithelial cell or an adipocyte. In some embodiments, the at least one cellular feature predicts the presence of a cancer cell or a cell in the tumor microenvironment. In some embodiments, the at least one cellular feature predicts the presence of a normal cell. In some embodiments, the at least one cellular feature is present on the same cell in tissue. In some embodiments, the at least one cellular feature is present on at least two different cells in tissue. In some embodiments, the at least one cellular feature is within a distance or between cell distances of the at least two different cells. In some embodiments, the at least one cellular feature is present on a tissue structure in a normal or a diseased state. In some embodiments, the diseased state comprises a cancer diseased state, an immunological diseased state, a neurological diseased state, an antiviral diseased state, a cardiovascular diseased state, or an autoimmune diseased state. In some embodiments, the tissue structure comprises epithelium, a duct, or a blood vessel. In some embodiments, the at least one cellular feature comprises a change in expression of an extracellular molecule. In some embodiments, the at least one cellular feature comprises a change in a glycosylation pattern of an extracellular molecule. In some embodiments, the at least one cellular feature comprises a change in enzymatic activity of an extracellular molecule. In some embodiments, the at least one cellular feature comprises a change in intracellular or intercellular communication. In some embodiments, the at least one cellular feature comprises a change in intracellular or intercellular signaling or communication. In some embodiments, the at least one cellular feature comprises a change in cell adhesion, mechanics or movement. In some embodiments, the at least one cellular feature comprises tissue localization of an extracellular molecule. In some embodiments, the tissue localization comprises a diseased tissue or tumor microenvironment. In some embodiments, the tissue localization comprises a cancer structure or tumor microenvironment. In some embodiments, the extracellular molecule internalizes when contacted by the antibody. In some embodiments, different samples are assessed for common cellular features. In some embodiments, different samples are assessed for different cellular features. In some embodiments, the method further comprises characterizing the at least one cellular feature based on an intensity of an imaging agent in the image. In some embodiments, the at least one cellular feature is assessed after administration of at least one ligand. In some embodiments, the at least one cellular feature is assessed after administration of at least one therapeutic or at least one therapeutic regimen.

In some embodiments, the super-resolution microscope comprises a deterministic super resolution microscope. In some embodiments, the super-resolution microscope is a stimulated emission depletion (STED) microscope or a ground state depletion (GSD) microscope. In some embodiments, the super-resolution microscope comprises a stochastic super-resolution microscope. In some embodiments, the super-resolution microscope is a MINFLUX microscope. In some embodiments, the super-resolution microscope is a stochastic optical reconstruction microscopy (STORM) microscope. In some embodiments, the imaging comprises tracking the one or more ligands, therapeutics, or cell surface receptors over a period of time.

In some embodiments, the cell comprises a fixed cell. In some embodiments, the cell is fixed in tissue. In some embodiments, the cell is in live tissue. In some embodiments, the cell comprises a live cell. In some embodiments, the cell comprises a mammalian cell. In some embodiments, the disease state cells are derived from a tumor cell line. In some embodiments, the normal state cells are derived from a normal cell line. In some embodiments, the cell is imaged on a tissue microarray, coverslip, flow cell, chip, or microfluidics chamber. In some embodiments, the tissue microarray, coverslip, flow cell, chip, or microfluidics chamber comprises frozen tissue. In some embodiments, the tissue microarray, coverslip, flow cell, chip, or microfluidics chamber comprises live tissue. In some embodiments, the tissue microarray, coverslip, flow cell, chip, or microfluidics chamber comprises fixed tissue. In some embodiments, the tissue microarray coverslip, flow cell, chip, or microfluidics chamber is formed via a laser microdissection process.

In some embodiments, the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc. In some embodiments, the antibody is a bispecific antibody. In some embodiments, the antibody is a multispecific antibody. In some embodiments, the antibody comprises at least one, at least two, at least three, at least four, at least five, or at least six VHH domains or more. In some embodiments, the antibody comprises at least one, at least two, at least three, at least four, at least five, or at least six VL domains or more. In some embodiments, the antibody comprises at least one, at least two, at least three, at least four, at least five, or at least six VH domains or more. In some embodiments, the antibody is an immune cell engager. In some embodiments, the antibody comprises an effector domain (e.g., CD3 or CD16A). In some embodiments, the antibody-based therapy is on a CAR-T cell or myeloid cell or NK cell or other cell-based therapy. In some embodiments, the antibody is in a vaccine. In some embodiments, the antibody is a translated nucleic acid strand or strands. In some embodiments, the antibody comprises a single chain polypeptide. In some embodiments, the antibody comprises a homodimer. In some embodiments, the antibody comprises a heterodimer.

In some embodiments the database of imaged cellular features are imaged at a resolution of at least about 100 nanometers. In some embodiments, the database of imaged cellular features are imaged at a resolution of at least about 50 nanometers. In some embodiments, the database of imaged cellular features are imaged at a resolution of at least about 40 nanometers. In some embodiments, the database of imaged cellular features are imaged at a resolution of at least about 30 nanometers. In some embodiments, the database of imaged cellular features are imaged at a resolution of at least about 20 nanometers. In some embodiments, the database of imaged cellular features are imaged at a resolution of at least about 10 nanometers. In some embodiments, the database of imaged cellular features are imaged at a resolution of at least about 9 nanometers. In some embodiments, the database of imaged cellular features are imaged at a resolution of at least about 8 nanometers. In some embodiments, the database of imaged cellular features are imaged at a resolution of at least about 7 nanometers. In some embodiments, the database of imaged cellular features are imaged at a resolution of at least about 6 nanometers. In some embodiments, the database of imaged cellular features are imaged at a resolution of at least about 5 nanometers. In some embodiments, the database of imaged cellular features are imaged at a resolution of at least about 4 nanometers. In some embodiments, the database of imaged cellular features are imaged at a resolution of at least about 3 nanometers. In some embodiments, the database of imaged cellular features are imaged at a resolution of at least about 2 nanometers. In some embodiments, the database of imaged cellular features are imaged at a resolution of at least about 1 nanometers. In some embodiments, the database of imaged cellular features are imaged at a single molecule resolution. In some embodiments, the database of imaged cellular features are imaged at a single fluorophore resolution.

In some embodiments, the database of imaged cellular features are imaged in a high throughput format. In some embodiments, the high throughput format comprises use of at least one 96 well plate. In some embodiments, the high throughput format comprises use of at least one 192 well plate. In some embodiments, the high throughput format comprises use of at least one 384 well plate. In some embodiments, the high throughput format comprises use of at least one 1536 well plate. In some embodiments, the high throughput format comprises the use of slides, coverslips, or flow cells containing multiple channels. In some embodiments, multiple cellular features are imaged at substantially the same time. In some embodiments, multiple cellular features are imaged sequentially. In some embodiments, multiple cellular features are imaged at substantially the same time and others are imaged sequentially. In some embodiments, the multiple cellular features are on surfaces of multiple cells. In some embodiments, imaging of a cellular feature is completed in less than about 5 minutes. In some embodiments, imaging the database of imaged cellular features is done autonomously.

In some embodiments, the extracellular molecule is a membrane-bound protein, or a ligand bound to a membrane. In some embodiments, the extracellular molecule is selected from a protein ligand, a sugar, a lipid, a ligand, an extracellular receptor, a membrane-bound protein, a soluble protein, a structural protein, and a protein or ligand bound to a membrane protein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a computer system that is programmed or otherwise configured to implement methods provided herein;

FIG. 2 shows an example of an application of the present disclosure wherein spatially-localized K_{d}s for one or more binding molecules (e.g., VHHs) binding to a cell surface are derived from single molecule tracking (SMT) experiments and then matched to cellular features in an image database;

FIG. 3 shows a stimulated emission depletion (STED) image of A549 cells stained with labeled anti-TROP2 VHH binder 1 (TROP2_1), wherein the image is 34.68×17.04 micrometers (μm) (1734×852 pixels) with a resolution of 20 nanometers (nm)/pixel;

FIG. 4 shows a STED image of A549 cells stained with labeled TROP2_1, wherein the image is 34.68×17.04 μm (1734×852 pixels) with a resolution of 20 nm/pixel;

FIGS. 5A-5B show SKBR3 cells stained with labeled TROP2_1 (FIG. 5A) and labeled anti-HER2 VHH binder 3 (HER2_3) (FIG. 5B), wherein the images are 34.60×43.06 μm (1730×2153 pixels) with a resolution of 20 nm/pixel; and

FIGS. 6A-6B show MCF7 cells stained with labeled TROP2_1 (FIG. 6A) and labeled HER2_3 (FIG. 6B), wherein the images are 63.86×58.30 μm (3193×2915 pixels) with a resolution of 20 nm/pixel.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

### Definitions

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

The terms “polypeptide” and “protein” are used interchangeably to refer to a polymer of amino acid residues, and are not limited to a minimum length. Polypeptides, including the provided polypeptides and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the polypeptides provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, a “antigen binding domain” or “antigen interacting domain” refers to an immunoglobulin derivative with antigen binding properties, i.e. immunoglobulin polypeptides or fragments thereof that contain an antigen binding site. In some cases, the binding domain comprises a variable domain of an antibody or fragments thereof. Each antigen-binding domain is formed by an antibody, i.e. immunoglobulin, variable heavy chain domain (VH) and an antibody variable light chain domain (VL) binding to the same epitope, whereas the variable heavy chain domain (VH) comprises three heavy chain complementarity determining regions (CDR): CDR1, CDR2 and CDR3; and the variable light chain domain (VL) comprises three light chain complementarity determining regions (CDR): CDR1, CDR2 and CDR3. In some instances, the binding domain according to some embodiments herein is devoid of immunoglobulin constant domains. In some instances, the variable light and heavy chain domains forming the antigen binding site is covalently linked with one another, e.g. by a peptide linker, or in other instances, the variable light and heavy chain domains non-covalently associate with one another to form the antigen binding site. Binding domains and interacting domains also refer to antibody fragments or antibody derivatives including, for example, Fab, Fab′, F(ab′)2, Fv fragments, single-chain Fv, tandem single-chain Fv((scFv)2, Bi-specific T-cell engagers (BiTE®), dual affinity retargeting antibodies (DART™), diabody, DuoBody® IgG molecules, single domain antibodies (e.g., VHH), TriTacs, and the like. Furthermore, in certain instances, the binding domain is multivalent, i.e. has two, three or more binding sites for one or more antigens.

In certain embodiments, the antigen binding or antigen interacting domains comprise sequences derived from the complementarity determining regions of antibodies. The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January;27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8;309 (3): 657-70, (“Aho” numbering scheme); and Whitelegg NR and Rees AR, “WAM: an improved algorithm for modelling antibodies on the WEB,” Protein Eng. 2000 December; 13 (12): 819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

In certain embodiments, the antigen binding or antigen interacting domains comprise sequences derived from the complementarity determining regions of antibodies. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

In certain embodiments, the antigen binding or antigen interacting domains are “humanized.” A “humanized” polypeptide or antibody is is one in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided polypeptides and antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. Though described herein as an antibody-based therapy, in some embodiments, the methods and systems of the present disclosure are used to image interactions between non-therapeutic antibodies or antibody derivatives (e.g., portions of antibodies) with a cell. For example, a fluorophore is coupled to a VHH domain and used to map the surface properties and cellular features of the cell. In this example, the surface properties and cellular features are compared between diseased and normal cells.

Non-limiting examples of a fluorophore or imaging agent includes, but are not limited to, KK114 (also known as Abberior STAR RED), Abberior STAR ORANGE, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, Oregon Green, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5-(or 6-) iodoacetamidofluorescein, 5- {[2 (and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, quantum dots, or other fluorophores.

The polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

The nucleic acids encoding the polypeptides described herein can be used to infect, transfect, transform, or otherwise render a suitable cell transgenic for the nucleic acid, thus enabling the production of polypeptide for commercial or therapeutic uses. Standard cell lines and methods for the production of antibodies or polypeptides from a large-scale cell culture are known in the art. See e.g., Li et al., “Cell culture processes for monoclonal antibody production.” Mabs. 2010 September-Oct; 2 (5): 466-477. In certain embodiments, the cell is a Eukaryotic cell. In certain embodiments, the Eukaryotic cell is a mammalian cell. In certain embodiments, the mammalian cell is a cell line useful for producing polypeptides or antibodies is a Chines Hamster Ovary cell (CHO) cell, an NSO murine myeloma cell, or a PER.C6® cell. In certain embodiments, the nucleic acid encoding the antibody is integrated into a genomic locus of a cell useful for producing polypeptides or antibodies. In certain embodiments, described herein is a method of making an antibody comprising culturing a cell comprising a nucleic acid encoding an antibody under conditions in vitro sufficient to allow production and secretion of said antibody.

Though the methods and systems described herein, in some embodiments, are described for use in disease-state differentiation, therapeutic design, and identification of therapeutic targets, the methods and systems described herein can be used in a variety of applications. For example, the databases, methods, and systems described herein can be used for patient selection (e.g., selection of a cancer type or study group). In another example, the databases, methods, and systems described herein can be used in combo therapy selection (e.g., selection of a combination of agents). In another example, the databases, methods, and systems described herein can be used for drug discovery (e.g., discovery of new targets or therapeutic compounds). In some embodiments, a plurality of extracellular receptors described herein is a plurality of the same extracellular receptor. In some embodiments, a plurality of extracellular receptors described herein is a plurality of different extracellular receptors. In another example, the databases, methods, and systems described herein can be used in determining a therapeutic can be repurposed for a different application (e.g., determination of a viability of an approved therapeutic for use in a different application from the application the therapeutic was approved for).

Though described herein with reference to antibody fragments or derivatives, the databases, methods, and systems of the present disclosure can be used to study the effects of full antibodies on a sample. For example, an antibody can be contacted to a sample and the cellular features of the sample can be recorded in a database. Examples of antibodies include, but are not limited to, IgA, IgD, IgE, IgG, IgM, IgT/Z, IgY, IgW, or the like. In some embodiments, the antibody is an antibody associated with a CAR T cell. In some embodiments the antibody is an antibody-drug conjugate. In some embodiments, a ribonucleic acid (RNA) aptamer is used in place of an antibody. In some embodiments, a natural receptor or ligand is used in place of an antibody.

As used herein, a molecular signature or a molecular fingerprint is defined as all or part of the configuration or states of one extracellular molecule on a cell surface comprised of at least one cellular feature. As used herein, a cell signature or cell fingerprint is defined as all or part of the configuration or states of two or more extracellular molecules on the cell surface comprised of at least one cellular feature. As used herein, a tissue signature or tissue fingerprint is defined as a molecular signature or fingerprint or cell signature or fingerprint on the surface of two or more cells (or collection of cells) in the tissue or sample.

### Methods of Generating a Database Comprising Cellular Features

Cancer cells are morphologically and functionally different from normal cells, and different cell signaling pathways are operative. Extracellular receptors may cluster in lipid rafts (or otherwise look different) during cell signaling and amplify the signal inside the cell. Certain features of extracellular receptors may differ between cancer and normal cells. In an aspect of the present disclosure, super-resolution microscopy (SRM) at scale and pharmaceutically relevant binders to known cancer antigens are used to build a high content normal/cancer cell (tissue) database. This may be directly translatable to a therapeutic strategy and may be used across research and development. The database may be used for target selection and validation (and new target discovery) for monospecific, bispecific, and multispecific therapeutics, drug design and validation, selection of combination therapies for clinical trials, cancer indication selection for clinical trials, and patient selection for clinical trials. Features are characterized in terms of whether the feature is present and, if so, determining its location, nearest neighbors, whether it is internalized, etc.

In some embodiments, the present disclosure provides a database of cellular interactions that can be used in a variety of different applications. In some embodiments, the database provides new information about the interaction of a therapy (e.g., an antibody-based therapy, a small molecule therapy, etc.) with both diseased and healthy cells. In some embodiments, the information can then be used to better target different types of cells, thus improving the efficacy of the screened therapies. In some embodiments, the information of the database can be used to characterize features in diseased and non-diseased cells, and subsequently interpreted for patterns that provide information regarding the diseased-state and non-diseased-state cells. In some embodiments, the information of the database is used for target selection, drug design, lead and development candidate selection, patient selection for clinical trials, and/or indication selection.

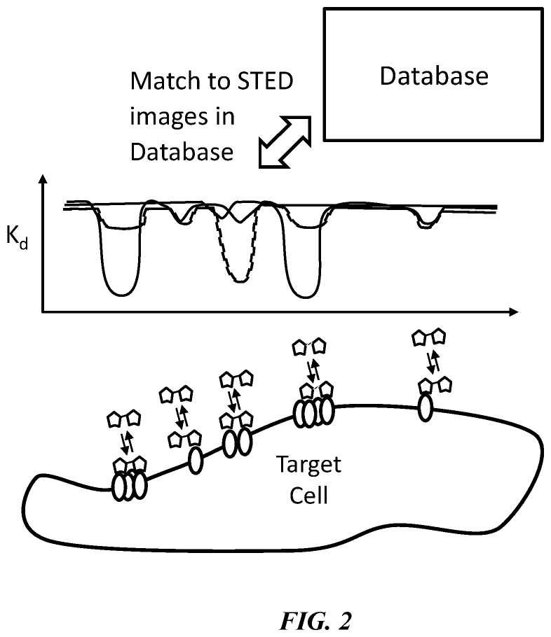

In another aspect of the present disclosure, spatially-localized affinities (Kas) for T cell engagers (TCEs) or other binding molecules (e.g., antibody-based therapies) to a cell surface are derived by single molecule tracking (SMT) experiments and matched to cellular features of the cell in the database to create a two-dimensional (2D) K_{d} profile for the cell surface in the database (see FIG. 2). SMT allows measuring of on-rates (kon) and off-rates (koff) of binding molecules including TCEs. This allows determining a spatially localized affinity (K_{d}=koff/kon) of a binding molecule or TCE on the surface of the cell (e.g., cancer cell or other cell). If the binding molecule or TCE includes repetitive single variable domain on a heavy chain (VHHs) and/or two or more different VHHs that bind different targets on the cell surface, then there will be an avidity effect if those targets cluster to form multimers. Hence, whenever targets cluster to form structures that are recognized by the binding molecule/TCE, then there is a higher affinity (lower K_{d}) at that location on the cell. Using the special K_{d} pattern and matching it to the cell surface target pattens (cellular features) of the cell observed in SRM images, one can then predict the binding behavior of the binding molecule/TCE to the surface of a separate cell or tissue by recording SRM images of the separate cell/tissue in the presence of the binding molecule/TCE, assuming the target presentation/cellular features are substantially the same for both cells/tissues. This strategy may be valuable for predicting the binding behavior of binding molecules/TCEs to normal vs. cancer cells or tissues, for example.

Disclosed herein are methods of generating a database comprising cellular features from a disease-state cell and non-disease state cell comprising imaging a sample that has been contacted by an antibody using a super-resolution microscope and generating a database comprising at least one cellular feature of a cell in the sample and determining if the at least one cellular feature is indicative of the disease-state cell or the non-disease-state cell.

### Cellular Feature

In some embodiments, the at least one cellular feature comprises an image of an extracellular molecule. In some embodiments, the at least one cellular feature comprises two- or three-dimensional coordinates of an extracellular molecule. In some embodiments, the at least one cellular feature comprises intensities for an extracellular molecule. In some embodiments, the at least one cellular feature comprises all or part of a molecular fingerprint, molecular signature, or map of the positions for an extracellular molecule. In some embodiments, the at least one cellular feature comprises all or part of a schematic or topical representation of an extracellular molecule on a cell or tissue. In some embodiments, the at least one cellular feature comprises proximity between two or more extracellular molecules. In some embodiments, the two or more extracellular molecules are of the same target or different targets. In some embodiments, the at least one cellular feature comprises interactions of at least two extracellular molecules. In some embodiments, the at least one cellular feature comprises a distance or distances between two or more extracellular molecules. In some embodiments, the at least one cellular feature comprises an individual extracellular molecule or pairs of extracellular molecules. In some embodiments, the individual or pairs of extracellular molecules are adjacent or in contact with each other. In some embodiments, the individual or pairs of extracellular molecules are within a distance of about 1 nanometer of each other. In some embodiments, the individual or pairs of extracellular molecules are with a distance of about 1 nanometer to about 100 nanometers of each other. In some embodiments, the at least one cellular feature comprises individual, pairs, or triplets of extracellular molecules. In some embodiments, the individual, pairs, or triplets of extracellular molecules are adjacent or in contact with each other. In some embodiments, the individual, pairs, or triplets of extracellular molecules are within a distance of about 1 nanometer of each other. In some embodiments, the individual, pairs, or triplets of extracellular molecules are within a distance of about 1 nanometer to about 100 nanometers of each other. In some embodiments, the at least one cellular feature comprises pairs, triplets, quadruplets, quintuplets, or higher order clusters of extracellular molecules. In some embodiments, the pairs, triplets, quadruplets, quintuplets or higher order clusters or extracellular molecules are adjacent or in contact with each other. In some embodiments, the pairs, triplets, quadruplets, quintuplets or higher order clusters of extracellular molecules comprise the same or different extracellular molecules. In some embodiments, the pairs, triplets, quadruplets, quintuplets, or higher order clusters or extracellular molecules are within a distance of about 1 nanometer of each other. In some embodiments, the pairs, triplets, quadruplets, quintuplets, or higher order clusters of extracellular molecules are within a distance of about 1 nanometer to about 100 nanometers of each other. In some embodiments, the at least one cellular feature comprises a geometric relationship between two or more extracellular molecules. In some embodiments, the at least one cellular feature comprises movement of at least one extracellular molecule. In some embodiments, the movement is internalization or movement to an extracellular surface. In some embodiments, the at least one cellular feature comprises internalization of at least one extracellular molecule. In some embodiments, the at least one cellular feature comprises a change in two- or three-dimensional coordinates of an extracellular molecule. In some embodiments, the at least one cellular feature comprises a change in clustering of an extracellular molecule. In some embodiments, the at least one cellular feature comprises a change in cluster formation of an extracellular molecule. In some embodiments, the at least one cellular feature comprises an oligomerization state of an extracellular molecule. In some embodiments, the at least one cellular feature comprises a clustering state of an extracellular molecule. In some embodiments, the at least one cellular feature comprises the names of proteins clustering with an extracellular molecule. In some embodiments, an extracellular molecule is an extracellular molecule listed in Table 1.

TABLE 1
Exemplary Extracellular Molecules

| | Target ID |
|---|---|
| Cancer Targets | Number |
| Cadherin 6 (CDH6) | 1 |
| Neural cell adhesion molecule (NCAM) (NCAM1) (CD56) | 2 |
| 4-1BB ligand (4-1BBL) (TNFSF9) (CD137L) | 3 |
| Activated leukocyte cell adhesion molecule (ALCAM) (CD166) | 4 |
| Activin receptor-like kinase 1 (ACVRL1) (ALK1) (HHT2) | 5 |
| ADAM metallopeptidase domain 9 (ADAM9) | 6 |
| Alpha fetoprotein (AFP) | 7 |
| Angiopoietin 2 (ANG2) (ANGPT2) | 8 |
| Angiotensin II type 1 (AT1) receptor (AGTR1) | 9 |
| Anti-Mullerian hormone receptor type II (AMHR2) | 10 |
| AXL receptor tyrosine kinase (AXL) (UFO) | 11 |
| B and T lymphocyte attenuator (BTLA) | 12 |
| B7-H3 (CD276) | 13 |
| BLyS (BAFF) receptors | 14 |
| Bone morphogenetic protein receptors | 15 |
| Butyrophilin subfamily 2 member A2 (BTN2A2); Butyrophilin subfamily 2 | 16 |
| member A1 (BTN2A1) | |
| Butyrophilin like 2 (BTNL2) | 17 |
| Butyrophilin subfamily 3 member A1 (BTN3A1) | 18 |
| CA 125 | 19 |
| CA19.9 (sialyl Lewis A) | 20 |
| Cadherin 17 (CDH17); Tumor necrosis factor (TNF) receptor superfamily member | 21 |
| 10b (TNFRSF10B) (DR5) (TRAILR2) (CD262) | |
| Calreticulin (CALR) | 22 |
| Cancer/testis antigen 1B (NY-ESO-1) (CTAG1B) | 23 |
| Carbonic anhydrase IX (CAIX) | 24 |
| Carcinoembryonic antigen (CEA)-related cell adhesion molecule 1 (CEACAM1) | 25 |
| (CD66a) | |
| Carcinoembryonic antigen (CEA)-related cell adhesion molecule 5 (CEACAM5) | 26 |
| (CD66e) | |
| Carcinoembryonic antigen (CEA)-related cell adhesion molecule 6 (CEACAM6) | 27 |
| (NCA) (CD66c) | |
| CC chemokine receptor 2 (CCR2) (CD192); CC chemokine receptor 5 (CCR5) | 28 |
| (CD195) | |
| CC chemokine receptor 4 (CCR4) (CD194) | 29 |
| CC chemokine receptor 5 (CCR5) (CD195) | 30 |
| CC chemokine receptor 8 (CCR8) | 31 |
| CD160 | 32 |
| CD19 | 33 |
| CD20 | 34 |
| CD200 | 35 |
| CD22 (SIGLEC2) | 36 |
| CD24 | 37 |
| CD248 endosialin (TEM1) | 38 |
| CD27 | 39 |
| CD28 | 40 |
| CD3 | 41 |
| Carcinoembryonic antigen (CEA) | 42 |
| Silver homolog (SILV) (PMEL17) (GP100) (PMEL) | 43 |
| Epidermal growth factor receptor (EGFR) (ErbB1) (HER1) | 44 |
| Epidermal growth factor receptor 2 (HER2) (EGFR2) (ErbB2) (neu) | 45 |
| Epidermal growth factor receptor variant III (EGFRvIII) | 46 |
| FMS-like tyrosine kinase 3 (FLT3) (CD135) | 47 |
| G protein-coupled receptor family C group 5 member D (GPRC5D) | 48 |
| Guanylate cyclase C (GCC) (GUCY2C) | 49 |
| Interleukin-3 (IL-3) receptor alpha (CD123) | 50 |
| Melanoma-associated antigen A4 (MAGEA4) | 51 |
| Mucin 17, cell surface associated (MUC17) (MUC3) | 52 |
| Programmed cell death 1 ligand 1 (PD-L1) (B7-H1) (CD274) | 53 |
| Six-transmembrane epithelial antigen of prostate 1 (STEAP1) | 54 |
| Somatostatin receptor 2 (SSTR2) | 55 |
| Tumor necrosis factor (TNF) receptor superfamily member 17 (BCMA) | 56 |
| (TNFRSF17) (CD269) | |
| CD30 | 57 |
| Fc gamma receptor IIIa (CD16a) (FCGR3A) (FcgammaRIIIa) | 58 |
| CD33 (SIGLEC3) | 59 |
| CD37 | 60 |
| CD38 | 61 |
| CD3d molecule delta (CD3-TCR complex) (CD3D) | 62 |
| Survivin (BIRC5) | 63 |
| CD4 | 64 |
| CD40 | 65 |
| CD46 | 66 |
| CD47 | 67 |
| Mesothelin (MSLN) | 68 |
| CD5 | 69 |
| CD52 | 70 |
| CD55SC-1 | 71 |
| CD70 (CD27L) | 72 |
| CD74 molecule major histocompatibility complex class II invariant chain | 73 |
| CD79b molecule immunoglobulin-associated beta (CD79B) (B29) | 74 |
| CD9 | 75 |
| CD96 molecule (CD96) | 76 |
| CDKN1A interacting zinc finger protein 1 (CIZ1) | 77 |
| Claudin 1 (CLDN1) | 78 |
| Claudin 18 (CLDN18) | 79 |
| Claudin 3 (CLDN3) | 80 |
| Claudin 6 (CLDN6) | 81 |
| CLDN18.2 | 82 |
| Clusterin (CLU) (APOJ) | 83 |
| c-Mer proto-oncogene tyrosine kinase (MERTK) | 84 |
| TYRO3 protein tyrosine kinase (TYRO3) (Sky) | 85 |
| c-Met receptor tyrosine kinase (c-MET) (MET) (HGFR) | 86 |
| c-Met receptor tyrosine kinase (c-MET) (MET) (HGFR) | 87 |
| Collagen triple helix repeat containing 1 (CTHRC1) | 88 |
| Colony-stimulating factor 1 receptor (CSF1R) (C-FMS) (CD115) | 89 |
| Complement 5 (C5) | 90 |
| Complement 5a (C5a) | 91 |
| Complement factor H (factor H) (CFH) | 92 |
| Complement receptor 5A (C5AR1) (C5AR) (CD88) | 93 |
| Connective tissue growth factor (CTGF) | 94 |
| C-type lectin domain family 12 member A (CLEC12A) (CLL1) (CD371) | 95 |
| C-type lectin domain family 2 member D (CLEC2D) (LLT1) | 96 |
| CXC chemokine receptor 4 (CXCR4) (NPY3R) | 97 |
| CXC chemokine receptor 5 (CXCR5) | 98 |
| Cytotoxic T-lymphocyte associated protein 4 (CTLA-4) (CTLA4) (CD152) | 99 |
| Programmed cell death 1 (PD-1) (PDCD1) (CD279) | 100 |
| Cytotoxic T-lymphocyte associated protein 4 (CTLA-4) (CTLA4) (CD152) | 101 |
| Tumor necrosis factor (TNF) receptor superfamily member 4 (OX40) (TNFRSF4) | 102 |
| (CD134) | |
| Delta like canonical Notch ligand 3 (DLL3) | 103 |
| Delta like canonical Notch ligand 4 (DLL4) | 104 |
| Delta like non-canonical Notch ligand 1 (DLK1) | 105 |
| Dickkopf homolog 1 (DKK1) | 106 |
| Dickkopf homolog 2 (DKK2) | 107 |
| Dickkopf homolog 3 (DKK3) | 108 |
| Dipeptidyl peptidase-4 (DPP-4) (CD26) | 109 |
| Ecto-5′-nucleotidase (CD73) (NT5E) | 110 |
| Ectonucleoside triphosphate diphosphohydrolase 1 (CD39) (ENTPD1) | 111 |
| EPH receptor A2 (EPHA2) | 112 |
| EPH receptor A3 (EPHA3) | 113 |
| Epidermal growth factor (EGF) | 114 |
| Epidermal growth factor receptor (EGFR) (ErbB1) (HER1) | 115 |
| Erb-b2 receptor tyrosine kinase 3 (HER3) (ERBB3) (EGFR3) | 116 |
| Epidermal growth factor receptor (EGFR) (ErbB1) (HER1) | 117 |
| Fc gamma receptor III (CD16) (FCGR3) | 118 |
| Leucine-rich repeat-containing G protein-coupled receptor 5 (LGR5) (GPR49) | 119 |
| Mucin 1 (MUC1) (CD227) | 120 |
| Epidermal growth factor receptor 2 (HER2) (EGFR2) (ErbB2) (neu) | 121 |
| Angiopoietin 2 (ANG2) (ANGPT2) | 122 |
| Epidermal growth factor receptor variant III (EGFRvIII) | 123 |
| Epithelial cell adhesion molecule (EpCAM) | 124 |
| Epithelial membrane protein 2 (EMP2) | 125 |
| Erb-b2 receptor tyrosine kinase 3 (HER3) (ERBB3) (EGFR3) | 126 |
| Estrogen receptor alpha 36 | 127 |
| Factor X (FX); Factor IX | 128 |
| Fc gamma receptor (FCGR) | 129 |
| Signal regulatory protein alpha (SIRPA) (CD172a) (SHPS-1) | 130 |
| Fc gamma receptor IIb (FCGR2B) (CD32B) | 131 |
| Fc receptor-like 5 (FCRL5) (FCRH5) | 132 |
| Fibroblast activation protein (FAP) | 133 |
| Fibroblast growth factor (FGF) receptor 2 (FGFR2) (KGFR) (CD332) | 134 |
| Fibroblast growth factor (FGF) receptor 2 b isoform (FGFR2b) | 135 |
| Fibroblast growth factor 23 (FGF23) | 136 |
| Fibronectin extra ED-B domain isoform (FnEDB) (oncofetal fibronectin) | 137 |
| FMS-like tyrosine kinase 3 (FLT3) (CD135) | 138 |
| Folate receptor 1 (FOLR1) (FR-alpha) (FOLR) | 139 |
| Frizzled homolog 10 (FZD10) | 140 |
| Frizzled homolog 7 (FZD7) | 141 |
| Fucosylated GM1 ganglioside | 142 |
| Galectin-9 (LGALS9) | 143 |
| Ganglioside (GD3) | 144 |
| Ganglioside GD2 (GD2) | 145 |
| GDNF family receptor alpha like (GFRAL) | 146 |
| Globohexaosylceramide (globo-H) | 147 |
| Glucocorticoid-induced tumor necrosis factor receptor (TNFR)-related protein | 148 |
| (GITR) (TNFRSF18) | |
| Glypican 1 (GPC1) | 149 |
| Glypican 3 (GPC3) | 150 |
| GnRH/LHRH receptor (GNRHR) | 151 |
| Granulocyte macrophage colony-stimulating factor (GM-CSF) (CSF2) | 152 |
| Growth differentiation factor 15 (GDF15) | 153 |
| Heat shock protein 70 (Hsp70) | 154 |
| Heparin-binding EGF-like growth factor (HBEGF) (DTR) | 155 |
| Hepatocyte growth factor/scatter factor (HGF/SF) | 156 |
| Hyaluronidase 2 (HYAL2) | 157 |
| Immunoglobulin G1 (IgG1) | 158 |
| Immunoglobulin superfamily member 11 (IGSF11) | 159 |
| Immunoglobulin-like domain containing receptor 2 (ILDR2) | 160 |
| Inducible T cell co-stimulator (ICOS) | 161 |
| Programmed cell death 1 (PD-1) (PDCD1) (CD279) | 162 |
| Insulin-like growth factor-1 (IGF-1) receptor (IGF1R) (CD221) | 163 |
| Insulin-like growth factor-2 (IGF-2) | 164 |
| Integrin alpha(V) (CD51) | 165 |
| Integrin alpha(V)beta(6) | 166 |
| Integrin alpha(V)beta(8); Transforming growth factor (TGF) beta 1 (TGFB1) | 167 |
| Integrin beta(1) (CD29) | 168 |
| Integrin beta(6) | 169 |
| Interferon beta (IFNB1) | 170 |
| Interleukin-1 (IL-1) alpha | 171 |
| Interleukin-1 (IL-1) beta | 172 |
| Interleukin-1 (IL-1) receptor accessory protein (IL-1RAP) | 173 |
| Interleukin-11 (IL-11) receptor | 174 |
| Interleukin-12 (IL-12) | 175 |
| Interleukin-15 (IL-15) receptor alpha chain (IL-15RA) | 176 |
| Interleukin-2 (IL-2) receptor | 177 |
| Interleukin-2 (IL-2) receptor alpha chain (CD25) (IL2RA) | 178 |
| Interleukin-2 (IL-2) receptor gamma chain (IL2RG) (CD132) | 179 |
| Interleukin-2 (IL-2) receptor beta chain (IL2RB) (CD122) | 180 |
| Interleukin-2 (IL-2) receptor | 181 |
| Interleukin-3 (IL-3) receptor alpha (CD123) | 182 |
| Interleukin-6 (IL-6) | 183 |
| Interleukin-6 (IL-6) receptor (CD126) | 184 |
| Interleukin-7 (IL-7) receptor (CD127) | 185 |
| Interleukin-8 (IL-8) (CXCL8) | 186 |
| Kappa myeloma antigen (KMA) | 187 |
| Kidney associated antigen 1 (KAAG1) | 188 |
| Killer cell immunoglobulin-like receptor three domains long cytoplasmic tail 2 | 189 |
| (KIR3DL2) (CD158K) | |
| Killer cell immunoglobulin-like receptor two domains long cytoplasmic tail 1 | 190 |
| (KIR2DL1) | |
| Killer cell lectin like receptor G1 (KLRG1) | 191 |
| Killer cell lectin-like receptor subfamily C member 1 (NKG2A) (CD159a) | 192 |
| (KLRC1) | |
| Kita-kyushu lung cancer antigen 1 (KK-LC-1) (CT83) | 193 |
| KRAS proto-oncogene, GTPase (KRAS) (K-Ras) | 194 |
| Leucine-rich repeat containing 15 (LRRC15) | 195 |
| Leucine-rich repeat containing 32 (LRRC32) (GARP) | 196 |
| Leucine-rich repeat-containing G protein-coupled receptor 5 (LGR5) (GPR49) | 197 |
| Leukemia inhibitory factor (LIF) | 198 |
| Leukocyte immunoglobin-like receptor subfamily B member 4 (LILRB4) (CD85k) | 199 |
| (ILT3) | |
| Leukocyte immunoglobulin-like receptor subfamily B member 2 (LILRB2) (LIR2) | 200 |
| Low-density lipoprotein receptor-related protein 5 (LRP5) | 201 |
| Low-density lipoprotein receptor-related protein 6 (LRP6) | 202 |
| Lymphocyte antigen 6 complex locus E (Ly6E) | 203 |
| Lymphocyte-activation gene 3 (LAG3) (CD223) | 204 |
| Lysine-specific demethylase 1 (LSD1) (KDM1A) | 205 |
| Lysosomal-associated membrane protein 1 (LAMP1) (CD107a) | 206 |
| Macrophage stimulating 1 receptor c-Met-related tyrosine kinase (MST1R) (RON) | 207 |
| (CD136) (CDw136) | |
| Major histocompatibility complex class I G (HLA-G) | 208 |
| Major histocompatibility complex class II DR (HLA-DR) | 209 |
| Mannose receptor C type 2 (MRC2) (UPARAP) (ENDO180) | 210 |
| Matrix metalloproteinase 9 (MMP9) | 211 |
| Melanoma cell adhesion molecule (MCAM) (MUC18) (CD146) | 212 |
| Melanoma-associated antigen A4 (MAGEA4) | 213 |
| Melanoma-associated chondroitin sulfate proteoglycan (MCSP) (CSPG4) (HMW- | 214 |
| MAA) | |
| Mesothelin (MSLN) | 215 |
| MHC class I polypeptide-related sequence A (MICA) | 216 |
| MHC class I polypeptide-related sequence B (MICB) | 217 |
| Motile sperm domain containing 2 (MOSPD2) | 218 |
| Mucin 1 (MUC1) (CD227) | 219 |
| Mucin 16 (MUC16) (CA125) | 220 |
| Mucin 5AC oligomeric mucus/gel-forming (MUC5AC) | 221 |
| Natural cytotoxicity triggering receptor 1 (NCR1) (NKP46) (CD335) | 222 |
| Sialic acid binding Ig like lectin 9 (SIGLEC9) | 223 |
| Nectin-4 (PRR4) | 224 |
| Interleukin-2 (IL-2) receptor beta chain (IL2RB) (CD122) | 225 |
| Neonatal Fc receptor (FCRN) (FCGRT) | 226 |
| Netrin 1 (NTN1) | 227 |
| Neuropilin 1 (NRP1) | 228 |
| Neutrophil elastase (ELANE) (NE) (HLE) | 229 |
| Nicotinamide phosphoribosyltransferase (NamPRT) (NAMPT) | 230 |
| NLR family pyrin domain containing 3 (NLRP3) (NALP3) (CIAS1) | 231 |
| Non-functioning P2X7 receptor (nfP2X7) | 232 |
| Nucleolin | 233 |
| Oncofetal antigen 5T4 | 234 |
| OX40 ligand (OX40L) (CD134L) | 235 |
| P selectin glycoprotein ligand-1 (PSGL-1) (CD162) (SELPLG) | 236 |
| Pancreatic adenocarcinoma upregulated factor (PAUF) (ZG16B) | 237 |
| P-cadherin (CDH3) | 238 |
| Periostin (POSTN) | 239 |
| Phosphatidylserine | 240 |
| Phosphodiesterase-I beta (ENPP3) (NPP3) (CD203c) | 241 |
| Phosphoinositide 3-kinase (PI3K) | 242 |
| Placental growth factor (PGF) (PLGF) | 243 |
| Plasminogen activator urokinase receptor (PLAUR) (uPAR) (suPAR) | 244 |
| Platelet derived growth factor receptor A (PDGFRA) (PDGFR2) (CD140A) | 245 |
| Poliovirus receptor related immunoglobulin domain containing (PVRIG) (CD112R) | 246 |
| Polymorphic epithelial mucin (PEM) (MUC-1) | 247 |
| Pre-B lymphocyte gene 1 (VPREB1) | 248 |
| Adenosine A2A receptor (ADORA2A) (A2AR) (ADORA2) | 249 |
| CD47 | 250 |
| Cytotoxic T-lymphocyte associated protein 4 (CTLA-4) (CTLA4) (CD152) | 251 |
| Lymphocyte-activation gene 3 (LAG3) (CD223) | 252 |
| Vascular endothelial growth factor (VEGF) receptor 1 (FLT1) (VEGFR-1); | 253 |
| Vascular endothelial growth factor (VEGF) receptor 3 (FLT4) (VEGFR-3) | |
| Interleukin-2 (IL-2) receptor beta chain (IL2RB) (CD122) | 254 |
| Transforming growth factor (TGF) beta 1 (TGFB1) | 255 |
| Tumor necrosis factor (TNF) receptor superfamily member 4 (OX40) (TNFRSF4) | 256 |
| (CD134) | |
| T cell immunoglobulin and mucin domain 3 (TIM3) (HAVCR2) | 257 |
| Prolactin receptor (PRLR) | 258 |
| Prostate-specific membrane antigen (PSMA) (FOLH1) (GCPII) | 259 |
| Protein tyrosine kinase 7 (PTK7) (CCK4) | 260 |
| Protein tyrosine phosphatase receptor type C (PTPRC) (CD45) (CD45R) | 261 |
| Receptor activator of NF-kappa B ligand (RANKL) (TNFSF11) | 262 |
| Receptor tyrosine kinase-like orphan receptor 1 (ROR1) | 263 |
| R-Spondin 3 (RSPO3) | 264 |
| S100 calcium binding protein A4 (S100A4) | 265 |
| S100 calcium binding protein A7 (S100A7) (psoriasin) | 266 |
| S100 calcium binding protein P (S100P) | 267 |
| Secretogranin III (SCG3) | 268 |
| Semaphorin 4D (SEMA4D) | 269 |
| Sialic acid binding Ig like lectin 15 (SIGLEC15) | 270 |
| Sialophorin (CD43) (SPN) | 271 |
| Signal regulatory protein alpha (SIRPA) (CD172a) (SHPS-1) | 272 |
| SLAM family member 7 (SLAMF7) (CS1) | 273 |
| Solute carrier family 3 member 2 (SLC3A2) (CD98) | 274 |
| Solute carrier family 34 sodium phosphate member 2 (SLC34A2) (NaPi2b) | 275 |
| Solute carrier family 39 zinc transporter member 6 (SLC39A6) (LIV-1) (ZIP6) | 276 |
| Solute carrier family 7 member 5 (SLC7A5) (LAT1) | 277 |
| Somatostatin receptor | 278 |
| Stabilin 1 (STAB1) (CLEVER-1) (FEEL-1) | 279 |
| Stage-specific embryonic antigen-4 (SSEA-4) | 280 |
| Stem cell factor (SCF) receptor tyrosine kinase (c-Kit) (KIT) (CD117) | 281 |
| Surface antigen in leukemia (SAIL) | 282 |
| T cell immunoglobulin and mucin domain 3 (TIM3) (HAVCR2) | 283 |
| T cell immunoreceptor with Ig and ITIM domains (TIGIT) | 284 |
| Tenascin C (TNC) (TN) | 285 |
| Thomsen-Friedenreich antigen (TF) | 286 |
| Tissue factor (TF) (CD142) (thromboplastin) | 287 |
| Tn antigen | 288 |
| Toll-like receptor 7 (TLR7) | 289 |
| Toll-like receptor 8 (TLR8) | 290 |
| Topoisomerase I (TOP1) | 291 |
| Tumor-associated calcium signal transducer 2 (TROP2) (TACSTD2) (EGP-1) | 292 |
| Transferrin receptor (TFRC) (TFR) (CD71) | 293 |
| Transforming growth factor (TGF) beta 1 (TGFB1) | 294 |
| Transforming growth factor (TGF) beta 1 (TGFB1) | 295 |
| Transforming growth factor (TGF) beta 2 (TGFB2) | 296 |
| Transforming growth factor (TGF) beta 3 (TGFB3) | 297 |
| Triggering receptor expressed on myeloid cells 1 (TREM1) | 298 |
| Triggering receptor expressed on myeloid cells 2 (TREM2) | 299 |
| Tumor necrosis factor (TNF) receptor superfamily member 10a (TNFRSF10A) | 300 |
| (DR4) (TRAILR1) (CD261) | |
| Tumor necrosis factor (TNF) receptor superfamily member 10b (TNFRSF10B) | 301 |
| (DR5) (TRAILR2) (CD262) | |
| Tumor necrosis factor (TNF) receptor superfamily member 17 (BCMA) | 302 |
| (TNFRSF17) (CD269) | |
| Fc gamma receptor IIIa (CD16a) (FCGR3A) (FcgammaRIIIa) | 303 |
| Tumor necrosis factor (TNF) receptor superfamily member 25 (TNFRSF25) (DR3) | 304 |
| (LARD) (TRAMP) (WSL-1) | |
| Tumor necrosis factor (TNF) receptor superfamily member 4 (OX40) (TNFRSF4) | 305 |
| (CD134) | |
| Tumor necrosis factor (TNF) receptor superfamily member 9 (4-1BB) (TNFRSF9) | 306 |
| (CD137) | |
| Tumor necrosis factor receptor 1 (TNFR1) (TNFRSF1A) (CD120a) | 307 |
| Tumor necrosis factor receptor 2 (TNFR2) (TNFRSF1B) | 308 |
| Tumor necrosis factor receptor superfamily member 13b (TNFRSF13B) (CD267) | 309 |
| (TACI) | |
| Tumor necrosis factor (TNF) receptor superfamily member 17 (BCMA) | 310 |
| (TNFRSF17) (CD269) | |
| Tumor necrosis factor receptor superfamily member 13c (BAFF-R) (TNFRSF13C) | 311 |
| Tumor-associated antigen GBU4-5 (GBU4-5) | 312 |
| Tumor-associated calcium signal transducer 2 (TROP2) (TACSTD2) (EGP-1) | 313 |
| Tumor-associated gp240 | 314 |
| Vascular endothelial growth factor (VEGF) | 315 |
| Vascular endothelial growth factor (VEGF) receptor 2 (VEGFR-2) (KDR/Flk-1) | 316 |
| Tyrosine kinase receptor 2 (Tie2) | 317 |
| Placental growth factor (PGF) (PLGF) | 318 |
| Vascular endothelial growth factor A (VEGF-A) | 319 |
| Neuropilin 1 (NRP1) | 320 |
| Vimentin (VIM) | 321 |
| V-region immunoglobulin-containing suppressor of T cell activation (VISTA) | 322 |
| V-set domain containing T cell activation inhibitor 1 (B7-H4) (VTCN1) | 323 |
| Wilms tumor 1 (WT1) | 324 |

In some embodiments, the at least one cellular feature is present on a cell in tissue. In some embodiments, the at least one cellular feature is present on a cell in cancer or on normal tissue. In some embodiments, the at least one cellular feature is present on a normal cell, on a cancer cell or both normal and cancer cells. In some embodiments, the at least one cellular feature is present on a normal cell, a cancer cell or a cell in the tumor microenvironment. In some embodiments, the at least one cellular feature is present on a cell in the tumor microenvironment. In some embodiments, the cell in the tumor microenvironment is selected from a B cell, a T cell, a Natural Killer (NK) cell, a myeloid cell, a fibroblast, and a pericyte. In some embodiments, the cell in the tumor microenvironment is selected from a stromal cell, an epithelial cell or an adipocyte. In some embodiments, the at least one cellular feature predicts the presence of a cancer cell or a cell in the tumor microenvironment. In some embodiments, the at least one cellular feature predicts the presence of a normal cell. In some embodiments, the at least one cellular feature is present on the same cell in tissue. In some embodiments, the at least one cellular feature is present on at least two different cells in tissue. In some embodiments, the at least one cellular feature is within a distance or between cell distances of the at least two different cells. In some embodiments, the at least one cellular feature is present on a tissue structure in a normal or a diseased state. In some embodiments, the diseased state comprises a cancer diseased state, an immunological diseased state, a neurological diseased state, an antiviral diseased state, a cardiovascular diseased state, or an autoimmune diseased state. In some embodiments, the tissue structure comprises epithelium, a duct, or a blood vessel. In some embodiments, the at least one cellular feature comprises a change in expression of an extracellular molecule. In some embodiments, the at least one cellular feature comprises a change in a glycosylation pattern of an extracellular molecule. In some embodiments, the at least one cellular feature comprises a change in enzymatic activity of an extracellular molecule. In some embodiments, the at least one cellular feature comprises a change in intracellular or intercellular communication. In some embodiments, the at least one cellular feature comprises a change in intracellular or intercellular signaling or communication. In some embodiments, the at least one cellular feature comprises a change in cell adhesion, mechanics or movement. In some embodiments, the at least one cellular feature comprises tissue localization of an extracellular molecule. In some embodiments, the tissue localization comprises a diseased tissue or tumor microenvironment. In some embodiments, the tissue localization comprises a cancer structure or tumor microenvironment. In some embodiments, the extracellular molecule internalizes when contacted by the antibody. In some embodiments, different samples are assessed for common cellular features. In some embodiments, different samples are assessed for different cellular features. In some embodiments, the method further comprises characterizing the at least one cellular feature based on an intensity of the imaging agent in the image. In some embodiments, the at least one cellular feature is assessed after administration of at least one ligand. In some embodiments, the at least one cellular feature is assessed after administration of at least one therapeutic or at least one therapeutic regimen.

### Super-Resolution Microscope

In some embodiments, the super-resolution microscope comprises a deterministic super resolution microscope. In some embodiments, the deterministic super-resolution microscopy uses light structuring to determine the sub-diffraction limit spatial information of the sample. For example, the deterministic super-resolution microscope can use a depletion ring to refine the excitation spot of the microscope. In some embodiments, the super-resolution microscope is a stimulated emission depletion (STED) microscope, a ground state depletion (GSD) microscope, saturated structed illumination microscopy (SSIM), super-resolution orthogonal deterministic imaging (SODI), or the like. In some embodiments, the super-resolution microscope is a MINFLUX microscope. For example, the super-resolution technique can be the MINFLUX technique developed by Abberior Instruments. MINFLUX is a super-resolution fluorescence microscopy method that can provide resolutions in the range of 1 to 3 nanometers (nm) by localizing individual switchable fluorophores with a probing donut-shaped excitation beam (see Gwosch et al., “MINFLUX nanoscopy delivers 3D multicolor nanometer resolution in cells”, Nature Methods, vol. 17, pp. 217-220, February 2020). In some embodiments, the super-resolution microscope uses a stochastic super-resolution technique (e.g., is a stochastic super-resolution microscope). In some embodiments, the stochastic super-resolution technique uses random chance to improve localization of emitters (e.g., by providing a flux such that only single emitters in a region are activated stochastically). In some embodiments, the super-resolution microscope is a stochastic optical reconstruction microscopy (STORM) microscope. In some embodiments, the stochastic super-resolution technique is direct stochastic optical reconstruction microscopy (dSTORM), photo-activated localization microscopy (PALM), fluorescence photo-activation localization microscopy (FPALM), spectral precision distance microscopy (SPDM), localization microscopy (e.g., single-molecule localization microscopy (SMLM)), cryogenic optical localization in three-dimensions (COLD), DNA-PAINT, or the like. In some embodiments, super-resolution microscopy or multiplexing methods as described by Jungmann et al. (U.S. Pat. No. 10,294,510B2 and EP 3027773B1) are used. In some embodiments, the imaging comprises tracking the antibody-based therapy or the cell surface receptor over a period of time.

To quantify extracellular proteins the fluorescence intensity observed can be normalized using the intrinsic molecular brightness of single fluorophore attached to the antibody. This can be done by labeling double strands of DNA with single fluorophores and attaching these to a small region of the cover slip used in imaging at a low concentration to ensure single molecules are spaced appropriately. During imaging an image is acquired of the small region using identical parameters as used for imaging cells and single molecule intensities from this region are used to calibrate the intensities acquired in the remaining image of the cells.

In some embodiments, the super-resolution microscope can be configured to take a time series of images of the sample. For example, the sample can be contacted with the antibody and imaged over time to provide a time series of the cellular feature. In this example, the time resolved data of the cellular feature provides additional data on the mechanism of action of the therapy as well as the efficacy. In some embodiments, the imaging comprises tracking the therapeutic, the cellular feature, or the cell surface receptors for a time period of at least about 0.001, 0.005, 0.01, 0.05, 0.1, 0.5, 1, 5, 10, 50, 100, 200, 300, 400, 500, 600, 700, 800, 900, 1,000, 5,000, 10,000, 50,000 or more milliseconds. In some embodiments, the imaging comprises tracking the therapeutic, the cellular feature, or the cell surface receptors for a time period of at most about 50,000, 10,000, 5,000, 1,000, 900, 800, 700, 600, 500, 400, 300, 200, 100, 50, 10, 5, 1, 0.5, 0.1, 0.05, 0.01, 0.005, 0.001, or fewer milliseconds.

### Cell/Tissue Microarray

In some embodiments, the cell comprises a fixed cell. In some embodiments, the cell is fixed in tissue. In some embodiments, the cell is in live tissue. In some embodiments, the cell comprises a live cell. In some embodiments, the cell comprises a mammalian cell. In some embodiments, the disease state cells are derived from a tumor cell line. In some embodiments, the normal state cells are derived from a normal cell line. In some embodiments, the cell is imaged on a tissue microarray, coverslip, flow cell, chip, or microfluidics chamber. In some embodiments, the tissue microarray, coverslip, flow cell, chip, or microfluidics chamber comprises frozen tissue. In some embodiments, the tissue microarray, coverslip, flow cell, chip, or microfluidics chamber comprises live tissue. In some embodiments, the tissue microarray, coverslip, flow cell, chip, or microfluidics chamber comprises fixed tissue. In some embodiments, the tissue microarray coverslip, flow cell, chip, or microfluidics chamber is formed via a laser microdissection process.

### Antibody

In some embodiments, the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc. In some embodiments, the antibody is a bispecific antibody. In some embodiments, the antibody is a multispecific antibody. In some embodiments, the antibody comprises at least one, at least two, at least three, at least four, at least five, or at least six VHH domains or more. In some embodiments, the antibody comprises at least one, at least two, at least three, at least four, at least five, or at least six VL domains or more. In some embodiments, the antibody comprises at least one, at least two, at least three, at least four, at least five, or at least six VH domains or more. In some embodiments, the antibody is an immune cell engager. In some embodiments, the antibody comprises an effector domain (e.g., CD3 or CD16A). In some embodiments, the antibody-based therapy is on a CAR-T cell or myeloid cell or NK cell or other cell-based therapy. In some embodiments, the antibody is in a vaccine. In some embodiments, the antibody is a translated nucleic acid strand or strands. In some embodiments, the antibody comprises a single chain polypeptide. In some embodiments, the antibody comprises a homodimer. In some embodiments, the antibody comprises a heterodimer.

### Resolution

In some embodiments, the database of imaged cellular features are imaged at or above (e.g., at a lower) a resolution of the super-resolution microscope. For example, a super-resolution microscope with a resolution limit of 5 nanometers can image a sample at a resolution of up to 5 nanometers. In some embodiments, the super-resolution microscope has a resolution of at least about 250, 225, 200, 175, 150, 125, 100, 90, 80, 70, 60, 50, 40, 30, 20, 10, 9, 8, 7, 6, 5, 4, 3, 2, 1, or less nanometers. In some embodiments, the super-resolution microscope has a resolution of at most about 1, 2, 3, 4, 5, 6, 7, 8, 9, 10, 20, 30, 40, 50, 60, 70, 80, 90, 100, 125, 150, 175, 200, 225, 250, or more nanometers. In some embodiments, the resolution of the super-resolution microscope is a range as defined by any two of the proceeding values. For example, in some embodiments the resolution of the super-resolution microscope is 1-50 nanometers.

In some embodiments, the database of imaged cellular features are imaged at a resolution of at least about 100 nanometers. In some embodiments, the database of imaged cellular features are imaged at a resolution of at least about 50 nanometers. In some embodiments, the database of imaged cellular features are imaged at a resolution of at least about 40 nanometers. In some embodiments, the database of imaged cellular features are imaged at a resolution of at least about 30 nanometers. In some embodiments, the database of imaged cellular features are imaged at a resolution of at least about 20 nanometers. In some embodiments, the database of imaged cellular features are imaged at a resolution of at least about 10 nanometers. In some embodiments, the database of imaged cellular features are imaged at a resolution of at least about 9 nanometers. In some embodiments, the database of imaged cellular features are imaged at a resolution of at least about 8 nanometers. In some embodiments, the database of imaged cellular features are imaged at a resolution of at least about 7 nanometers. In some embodiments, the database of imaged cellular features are imaged at a resolution of at least about 6 nanometers. In some embodiments, the database of imaged cellular features are imaged at a resolution of at least about 5 nanometers. In some embodiments, the database of imaged cellular features are imaged at a resolution of at least about 4 nanometers. In some embodiments, the database of imaged cellular features are imaged at a resolution of at least about 3 nanometers. In some embodiments, the database of imaged cellular features are imaged at a resolution of at least about 2 nanometers. In some embodiments, the database of imaged cellular features are imaged at a resolution of at least about 1 nanometers. In some embodiments, the database of imaged cellular features are imaged at a single molecule resolution. In some embodiments, the database of imaged cellular features are imaged at a single fluorophore resolution.

### High-Throughput Format

In some embodiments, the database of imaged cellular features are imaged in a high throughput format. In some embodiments, the high throughput format comprises use of at least one 96 well plate. In some embodiments, the high throughput format comprises use of at least one 192 well plate. In some embodiments, the high throughput format comprises use of at least one 384 well plate. In some embodiments, the high throughput format comprises use of at least one 1536 well plate. In some embodiments, the high throughput format comprises the use of slides, coverslips, or flow cells containing multiple channels. In some embodiments, multiple cellular features are imaged at substantially the same time. In some embodiments, multiple cellular features are imaged sequentially. In some embodiments, multiple cellular features are imaged at substantially the same time and others are imaged sequentially. In some embodiments, the multiple cellular features are on surfaces of multiple cells. In some embodiments, imaging of a cellular feature is completed in less than 5 minutes. In some embodiments, imaging the database of cellular features is done autonomously.

### Extracellular Molecule

In some embodiments, the extracellular molecule is a membrane-bound protein or a ligand bound to a membrane. In some embodiments, the extracellular molecule is selected from a protein ligand, a sugar, a lipid, a ligand, an extracellular receptor, a membrane-bound protein, a soluble protein, a structural protein, and a protein or ligand bound to a membrane protein.

### Samples

In some embodiments, a sample can be tested to determine if the sample has cellular features that are common with a second sample derived from the same source (e.g., patient) or a different source. In some embodiments, the assessment comprises one or more of genetic testing (e.g., nucleic acid detection or sequencing), protein testing (e.g., identification of a presence or absence of a predetermined protein), size testing (e.g., determining a size distribution of the cells present in the sample), label testing (e.g., determining a presence or absence of one or more labels on the cell), or the like, or any combination thereof. In some embodiments, the sample is assessed after administration of at least one therapeutic or at least one therapeutic regimen (e.g., an antibody-based therapeutic regimen, a small molecule-based therapeutic regimen, a biologic-based regimen, etc.). For example, the sample can be contacted with a therapeutic regimen and subsequently assessed in order to determine an effect of the regimen and if the sample is of value to fully characterize. In some embodiments, the sample is assessed after administration of a natural ligand or naturally occurring agent or cofactor. In some embodiments, pre-assessing the sample can reduce the full characterization of samples that do not display a property of interest (e.g., an impact or lack thereof of the therapeutic regimen), reducing wasted time and resources.

In some embodiments, the cell comprises a live cell. For example, the cell can be gathered from a living organism and used as gathered. In another example, the cell can be from a maintained cell line. In some embodiments, the cell is a eukaryotic cell. In some embodiments, the cell comprises a mammalian cell. In some embodiments, the mammalian cell is derived from a mouse, a rat, a rabbit, a dog, a cat, a horse, a cow, a sheep, a pig, a goat, a llama, an alpaca, a yak, a camel, or a human. In some embodiments, the cell comprises a non-living cell. In certain embodiments, the non-living cell is a dead cell. In some embodiments, the cell comprises a fixed cell. In some embodiments, the fixed cell is fixed as described elsewhere herein.

In some embodiments, the disease state cells are derived from a tumor cell line. Examples of tumor cell lines include, but are not limited to 3T3 cells, A549 cells, HeLa cells, Jurkat cells, and those available from commercial suppliers of cell lines such as, for example, MilliporeSigma, ThermoFischer, and the like.

In some embodiments, the cell is imaged on a tissue microarray. For example, the cell is a part of a tissue microarray and imaged as such. In some embodiments, the tissue microarray comprises a plurality to tissue samples contained within a single object. In some embodiments, the plurality of tissue samples is a plurality of different tissue samples. In some embodiments, the use of a tissue microarray enables screening of a variety of different tissue types in shorter time than individually screening each tissue type. In some embodiments, the tissue microarray comprises one or more of frozen tissue (e.g., tissue presently or previously preserved by freezing), live tissue (e.g., currently living tissue), fixed tissue (e.g., tissue which has been fixed in a matrix or otherwise preserved (e.g., by use of aldehydes, alcohols, oxidizing agents, etc.)), or the like, or any combination thereof. In some embodiments, the tissue microarray is formed via a laser microdissection process. In some embodiments, the tissue microarray is formed via a needle-coring process. In some embodiments, the tissue microarray is formed via a microtome process.

### Methods of Designing an Antibody-Based Therapy

Disclosed herein are methods of designing an antibody-based therapy comprising imaging an extracellular molecule on a cell for therapeutic intervention using a super-resolution microscope and characterizing a cellular feature of the imaged extracellular molecule and designing an antibody format based upon the characterizing of the cellular feature.

### Cellular Feature

In some embodiments, the cellular feature comprises an image of the extracellular molecule. In some embodiments, the cellular feature comprises two- or three-dimensional coordinates of the extracellular molecule. In some embodiments, the cellular feature comprises intensities for the extracellular molecule. In some embodiments, the cellular feature comprises all or part of a molecular fingerprint, molecular signature, or map of the positions for the extracellular molecule. In some embodiments, the cellular feature comprises all or part of a schematic or topical representation of an extracellular molecule on a cell or tissue. In some embodiments, the cellular feature comprises proximity between two or more extracellular molecules. In some embodiments, the two or more extracellular molecules are of the same target or different targets. In some embodiments, the cellular feature comprises interactions of at least two extracellular molecules. In some embodiments, the cellular feature comprises a distance or distances between two or more extracellular molecules. In some embodiments, the at least one cellular feature comprises an individual extracellular molecule or pairs of extracellular molecules. In some embodiments, the individual or pairs of extracellular molecules are adjacent or in contact with each other. In some embodiments, the individual or pairs of extracellular molecules are within a distance of about 1 nanometer of each other. In some embodiments, the individual or pairs of extracellular molecules are with a distance of about 1 nanometer to about 100 nanometers of each other. In some embodiments, the at least one cellular feature comprises individual, pairs, or triplets of extracellular molecules. In some embodiments, the individual, pairs, or triplets of extracellular molecules are adjacent or in contact with each other. In some embodiments, the individual, pairs, or triplets of extracellular molecules are within a distance of about 1 nanometer of each other. In some embodiments, the individual, pairs, or triplets of extracellular molecules are within a distance of about 1 nanometer to about 100 nanometers of each other. In some embodiments, the at least one cellular feature comprises pairs, triplets, quadruplets, quintuplets, or higher order clusters of extracellular molecules. In some embodiments, the pairs, triplets, quadruplets, quintuplets or higher order clusters or extracellular molecules are adjacent or in contact with each other. In some embodiments, the pairs, triplets, quadruplets, quintuplets or higher order clusters of extracellular molecules comprise the same or different extracellular molecules. In some embodiments, the pairs, triplets, quadruplets, quintuplets, or higher order clusters or extracellular molecules are within a distance of about 1 nanometer of each other. In some embodiments, the pairs, triplets, quadruplets, quintuplets, or higher order clusters of extracellular molecules are within a distance of about 1 nanometer to about 100 nanometers of each other. In some embodiments, the cellular feature comprises a geometric relationship between two or more extracellular molecules. In some embodiments, the cellular feature comprises movement of the extracellular molecule. In some embodiments, the movement is internalization or movement to an extracellular surface. In some embodiments, the cellular feature comprises internalization of the extracellular molecule. In some embodiments, the cellular feature comprises a change in two- or three-dimensional coordinates of the extracellular molecule. In some embodiments, the cellular feature comprises a change in clustering of the extracellular molecule. In some embodiments, the cellular feature comprises a change in cluster formation of the extracellular molecule. In some embodiments, the cellular feature comprises movement of at least one cellular receptor. In some embodiments, the cellular feature comprises an oligomerization state of an extracellular molecule. In some embodiments, the cellular feature comprises a clustering state of an extracellular molecule. In some embodiments, the cellular feature comprises the names of proteins clustering with an extracellular molecule. In some embodiments, the extracellular molecule comprises an extracellular molecule listed in Table 1. In some embodiments, the cellular feature is present on a cell in tissue. In some embodiments, the cellular feature is present on a cell in cancer or on normal tissue. In some embodiments, the cellular feature is present on a normal cell, on a cancer cell or both normal and cancer cells. In some embodiments, the cellular feature is present on a normal cell, a cancer cell or a cell in the tumor microenvironment. In some embodiments, the cellular feature is present on a cell in the tumor microenvironment. In some embodiments, the cell in the tumor microenvironment is selected from a B cell, a T cell, a Natural Killer (NK) cell, a myeloid cell, a fibroblast, and a pericyte. In some embodiments, the cell in the tumor microenvironment is selected from a stromal cell, an epithelial cell or an adipocyte. In some embodiments, the cellular feature predicts the presence of a cancer cell or a cell in the tumor microenvironment. In some embodiments, the cellular feature predicts the presence of a normal cell. In some embodiments, the cellular feature is present on the same cell in tissue. In some embodiments, the cellular feature is present on at least two different cells in tissue. In some embodiments, the cellular feature is within a distance or between cell distances of the at least two different cells. In some embodiments, the cellular feature is present on a tissue structure in a normal or a diseased state. In some embodiments, the diseased state comprises a cancer diseased state, an immunological diseased state, a neurological diseased state, an antiviral diseased state, a cardiovascular diseased state, or an autoimmune diseased state. In some embodiments, the cellular feature comprises a change in expression of the extracellular molecule. In some embodiments, the cellular feature comprises a change in a glycosylation pattern of the extracellular molecule. In some embodiments, the cellular feature comprises a change in enzymatic activity of an extracellular molecule. In some embodiments, the cellular feature comprises a change in intracellular or intercellular communication. In some embodiments, the cellular feature comprises a change in intracellular or intercellular signaling or communication. In some embodiments, the cellular feature comprises a change in cell adhesion, mechanics or movement. In some embodiments, the cellular feature comprises tissue localization of the extracellular molecule. In some embodiments, the tissue localization comprises a diseased tissue structure or microenvironment. In some embodiments, the tissue localization comprises a cancer structure or tumor microenvironment. In some embodiments, the extracellular molecule signals when contacted by the antibody-based therapy. In some embodiments, different samples are assessed for common cellular features. In some embodiments, different samples are assessed for different cellular features. In some embodiments, the method further comprises characterizing the cellular feature based on an intensity of the imaging agent in the image. In some embodiments, the cellular feature is assessed after administration of at least one ligand. In some embodiments, the cellular feature is assessed after administration of at least one at least one therapeutic or therapeutic regimen.

### Super-Resolution Microscopy

In some embodiments, the super-resolution microscope comprises a deterministic super resolution microscope. In some embodiments, the deterministic super-resolution microscopy uses light structuring to determine the sub-diffraction limit spatial information of the sample. For example, the deterministic super-resolution microscope can use a depletion ring to refine the excitation spot of the microscope. In some embodiments, the super-resolution microscope is a stimulated emission depletion (STED) microscope, a ground state depletion (GSD) microscope saturated structed illumination microscopy (SSIM), super-resolution orthogonal deterministic imaging (SODI), or the like. In some embodiments, the super-resolution microscope is a MINFLUX microscope. For example, the super-resolution technique can be the MINFLUX technique developed by Abberior Instruments. MINFLUX is a super-resolution fluorescence microscopy method that can provide resolutions in the range of 1 to 3 nanometers (nm) by localizing individual switchable fluorophores with a probing donut-shaped excitation beam (see Gwosch et al., “MINFLUX nanoscopy delivers 3D multicolor nanometer resolution in cells”, Nature Methods, vol. 17, pp. 217-220, February 2020). In some embodiments, the super-resolution microscope uses a stochastic super-resolution technique (e.g., is a stochastic super-resolution microscope). In some embodiments, the stochastic super-resolution technique uses random chance to improve localization of emitters (e.g., by providing a flux such that only single emitters in a region are activated stochastically). In some embodiments, the super-resolution microscope is a stochastic optical reconstruction microscopy (STORM) microscope. In some embodiments, the stochastic super-resolution technique is direct stochastic optical reconstruction microscopy (dSTORM), photo-activated localization microscopy (PALM), fluorescence photo-activation localization microscopy (FPALM), spectral precision distance microscopy (SPDM), localization microscopy (e.g., single-molecule localization microscopy (SMLM)), cryogenic optical localization in three-dimensions (COLD), DNA-PAINT, or the like. In some embodiments, super-resolution microscopy or multiplexing methods as described by Jungmann et al. (U.S. Pat. No. 10,294,510B2 and EP 3027773B1) are used. In some embodiments, the imaging comprises tracking the antibody-based therapy or the cell surface receptor over a period of time.

To quantify extracellular proteins the fluorescence intensity observed can be normalized using the intrinsic molecular brightness of single fluorophore attached to the antibody. This can be done by labeling double strands of DNA with single fluorophores and attaching these to a small region of the cover slip used in imaging at a low concentration to ensure single molecules are spaced appropriately. During imaging an image is acquired of the small region using identical parameters as used for imaging cells and single molecule intensities from this region are used to calibrate the intensities acquired in the remaining image of the cells.

In some embodiments, the super-resolution microscope can be configured to take a time series of images of the sample. For example, the sample can be contacted with the antibody-based therapy and imaged over time to provide a time series of the cellular feature. In this example, the time resolved data of the cellular feature provides additional data on the mechanism of action of the therapy as well as the efficacy. In some embodiments, the imaging comprises tracking the therapeutic, the cellular feature, or the cell surface receptors for a time period of at least about 0.001, 0.005, 0.01, 0.05, 0.1, 0.5, 1, 5, 10, 50, 100, 200, 300, 400, 500, 600, 700, 800, 900, 1,000, 5,000, 10,000, 50,000 or more milliseconds. In some embodiments, the imaging comprises tracking the therapeutic, the cellular feature, or the cell surface receptors for a time period of at most about 50,000, 10,000, 5,000, 1,000, 900, 800, 700, 600, 500, 400, 300, 200, 100, 50, 10, 5, 1, 0.5, 0.1, 0.05, 0.01, 0.005, 0.001, or fewer milliseconds.

### Cell/Tissue Microarray

In some embodiments, the cell comprises a fixed cell. In some embodiments, the cell is fixed in tissue. In some embodiments, the cell is in live tissue. In some embodiments, the cell comprises a live cell. In some embodiments, the cell comprises a mammalian cell. In some embodiments, the cell is imaged on a tissue microarray, coverslip, flow cell, chip, or microfluidics chamber. In some embodiments, the tissue microarray, coverslip, flow cell, chip, or microfluidics chamber comprises frozen tissue. In some embodiments, the tissue microarray, coverslip, flow cell, chip, or microfluidics chamber comprises live tissue. In some embodiments, the tissue microarray, coverslip, flow cell, chip, or microfluidics chamber comprises fixed tissue. In some embodiments, the tissue microarray, coverslip, flow cell, chip, or microfluidics chamber is formed via a laser microdissection process.

### Antibody

In some embodiments, the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc. In some embodiments, the antibody-based therapy is a bispecific antibody. In some embodiments, the antibody-based therapy is a multispecific antibody. In some embodiments, the antibody-based therapy comprises at least one, at least two, at least three, at least four, at least five, or at least six VHH domains or more. In some embodiments, the antibody-based therapy comprises at least one, at least two, at least three, at least four, at least five, or at least six VL domains or more. In some embodiments, the antibody-based therapy comprises at least one, at least two, at least three, at least four, at least five, or at least six VH domains or more. In some embodiments, the antibody-based therapy is an immune cell engager. In some embodiments, the antibody-based therapy comprises an effector domain (e.g., CD3 or CD16A). In some embodiments, the antibody-based therapy is on a CAR-T cell or myeloid cell or NK cell or other cell-based therapy. In some embodiments, the antibody-based therapy is in a vaccine. In some embodiments, the antibody-based therapy is a translated nucleic acid strand or strands. In some embodiments, the antibody-based therapy comprises a single chain polypeptide. In some embodiments, the antibody-based therapy comprises a homodimer. In some embodiments, the antibody-based therapy comprises a heterodimer. In some embodiments, the antibody-based therapy is present at a picomolar to nanomolar concentration. In some embodiments, the antibody-based therapy is present at a sub-picomolar concentration.

### Resolution

In some embodiments, the database of imaged cellular features are imaged at or above (e.g., at a lower) a resolution of the super-resolution microscope. For example, a super-resolution microscope with a resolution limit of 5 nanometers can image a sample at a resolution of up to 5 nanometers. In some embodiments, the super-resolution microscope has a resolution of at least about 250, 225, 200, 175, 150, 125, 100, 90, 80, 70, 60, 50, 40, 30, 20, 10, 9, 8, 7, 6, 5, 4, 3, 2, 1, or less nanometers. In some embodiments, the super-resolution microscope has a resolution of at most about 1, 2, 3, 4, 5, 6, 7, 8, 9, 10, 20, 30, 40, 50, 60, 70, 80, 90, 100, 125, 150, 175, 200, 225, 250, or more nanometers. In some embodiments, the resolution of the super-resolution microscope is a range as defined by any two of the proceeding values. For example, in some embodiments the resolution of the super-resolution microscope is 1-50 nanometers.

In some embodiments, the cellular feature is imaged at a resolution of at least about 100 nanometers. In some embodiments, the cellular feature is imaged at a resolution of at least about 50 nanometers. In some embodiments, the cellular feature is imaged at a resolution of at least about 40 nanometers. In some embodiments, the cellular feature is imaged at a resolution of at least about 30 nanometers. In some embodiments, the cellular feature is imaged at a resolution of at least about 20 nanometers. In some embodiments, the cellular feature is imaged at a resolution of at least about 10 nanometers. In some embodiments, the cellular feature is imaged at a resolution of at least about 9 nanometers. In some embodiments, the cellular feature is imaged at a resolution of at least about 8 nanometers. In some embodiments, the cellular feature is imaged at a resolution of at least about 7 nanometers. In some embodiments, the cellular feature is imaged at a resolution of at least about 6 nanometers. In some embodiments, the cellular feature is imaged at a resolution of at least about 5 nanometers. In some embodiments, the cellular feature is imaged at a resolution of at least about 4 nanometers. In some embodiments, the cellular feature is imaged at a resolution of at least about 3 nanometers. In some embodiments, the cellular feature is imaged at a resolution of at least about 2 nanometers. In some embodiments, the cellular feature is imaged at a resolution of at least about 1 nanometers. In some embodiments, the cellular feature is imaged at a single molecule resolution. In some embodiments, the cellular feature is imaged at a single fluorophore resolution.

### High-Throughput Format

Ins some embodiments, the cellular feature is imaged in a high throughput format. In some embodiments, the high throughput format comprises use of at least one 96 well plate. In some embodiments, the high throughput format comprises use of at least one 192 well plate. In some embodiments, the high throughput format comprises use of at least one 384 well plate. In some embodiments, the high throughput format comprises use of at least one 1536 well plate. In some embodiments, the high throughput format comprises the use of slides, coverslips, or flow cells containing multiple channels. In some embodiments, multiple cellular features are imaged at substantially the same time. In some embodiments, multiple cellular features are imaged sequentially. In some embodiments, multiple cellular features are imaged at substantially the same time and others are imaged sequentially. In some embodiments, the multiple cellular features are on surfaces of multiple cells. In some embodiments, imaging of a cellular feature is completed in less than about 5 minutes. In some embodiments, the imaging the cellular feature is done autonomously.

### Extracellular Molecule

In some embodiments, the extracellular molecule is a membrane-bound protein, or a ligand bound to a membrane. In some embodiments, the extracellular molecule is selected from a protein ligand, a sugar, a lipid, a ligand, an extracellular receptor, a membrane-bound protein, a soluble protein, a structural protein, and a protein or ligand bound to a membrane protein.

### Samples

In some embodiments, a sample can be tested to determine if the sample has cellular features that are common with a second sample derived from the same source (e.g., patient) or a different source. In some embodiments, the assessment comprises one or more of genetic testing (e.g., nucleic acid detection or sequencing), protein testing (e.g., identification of a presence or absence of a predetermined protein), size testing (e.g., determining a size distribution of the cells present in the sample), label testing (e.g., determining a presence or absence of one or more labels on the cell), or the like, or any combination thereof. In some embodiments, the sample is assessed after administration of at least one therapeutic or at least one therapeutic regimen (e.g., an antibody-based therapeutic regimen, a small molecule-based therapeutic regimen, a biologic-based regimen, etc.). For example, the sample can be contacted with a therapeutic regimen and subsequently assessed in order to determine an effect of the regimen and if the sample is of value to fully characterize. In some embodiments, the sample is assessed after administration of a natural ligand or naturally occurring agent or cofactor. In some embodiments, pre-assessing the sample can reduce the full characterization of samples that do not display a property of interest (e.g., an impact or lack thereof of the therapeutic regimen), reducing wasted time and resources.

In some embodiments, the cell comprises a live cell. For example, the cell can be gathered from a living organism and used as gathered. In another example, the cell can be from a maintained cell line. In some embodiments, the cell is a eukaryotic cell. In some embodiments, the cell comprises a mammalian cell. In some embodiments, the mammalian cell is derived from a mouse, a rat, a rabbit, a dog, a cat, a horse, a cow, a sheep, a pig, a goat, a llama, an alpaca, a yak, a camel, or a human. In some embodiments, the cell comprises a non-living cell. In certain embodiments, the non-living cell is a dead cell. In some embodiments, the cell comprises a fixed cell. In some embodiments, the fixed cell is fixed as described elsewhere herein.

In some embodiments, the disease state cells are derived from a tumor cell line. Examples of tumor cell lines include, but are not limited to 3T3 cells, A549 cells, HeLa cells, Jurkat cells, and those available from commercial suppliers of cell lines such as, for example, MilliporeSigma, ThermoFischer, and the like.

In some embodiments, the cell is imaged on a tissue microarray. For example, the cell is a part of a tissue microarray and imaged as such. In some embodiments, the tissue microarray comprises a plurality to tissue samples contained within a single object. In some embodiments, the plurality of tissue samples is a plurality of different tissue samples. In some embodiments, the use of a tissue microarray enables screening of a variety of different tissue types in shorter time than individually screening each tissue type. In some embodiments, the tissue microarray comprises one or more of frozen tissue (e.g., tissue presently or previously preserved by freezing), live tissue (e.g., currently living tissue), fixed tissue (e.g., tissue which has been fixed in a matrix or otherwise preserved (e.g., by use of aldehydes, alcohols, oxidizing agents, etc.)), or the like, or any combination thereof. In some embodiments, the tissue microarray is formed via a laser microdissection process. In some embodiments, the tissue microarray is formed via a needle-coring process. In some embodiments, the tissue microarray is formed via a microtome process.

### Therapeutic Design

In an aspect, the present disclosure provides a method of designing an antibody-based therapy comprising imaging a cell surface receptor on a cell for therapeutic intervention and characterizing a cellular feature of the imaged cell surface receptor, and identifying an antibody format based upon the characterizing of the cellular features.

In certain embodiments, disclosed herein, are antibodies useful for the treatment of a cancer or tumor. In some embodiments, the antibody-based therapy is useful for the treatment of an immunological, neurological, antiviral, cardiovascular, and/or autoimmune disease. Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will effect remission of a cancer being treated. In certain embodiments, treatment encompasses use as a prophylactic or maintenance dose intended to prevent reoccurrence or progression of a previously treated cancer or tumor. For treating bacterial or viral disease treatment includes, but is not limited to reducing one or more symptoms associated with the viral or bacterial disease, such as reducing fever, nausea, diarrhea, vomiting, sore-throat, cough, runny-nose, and/or rash. Treatment of bacterial or viral disease can reduce overall levels of virus or bacteria in the body, reduce a period which an individual can infect others, or reduce overall disease or convalescence time. Treatment of autoimmune or inflammatory diseases includes but is not limited to reduction in total or self-antibody levels, or reduction in total or self-cellular immune responses. Treatment can also be associate with specific symptoms of autoimmune disease related to an overzealous antibody or cellular immune response. Treatment of fibrotic disease may reduce or slow the appearance of fibrotic tissue or the deposition of collagen in the tissue. Treatment of cardiovascular disease may increase indicia of cardiovascular health including reducing blood pressure, reducing atherosclerotic lesions, or increasing heart function as indicated by the ability to pump blood. It is understood by those of skill in the art that not all individuals will respond equally, or at all, to a treatment that is administered, nevertheless these individuals are considered to be treated.

In certain embodiments, the single chain polypeptides are for use in treating a viral infection. In certain embodiments, the single chain polypeptides are for use in treating a bacterial infection. In certain embodiments, the single chain polypeptides are for use in treating a solid tumor cancer. In certain embodiments, the single chain polypeptides are for use in treating a hematological cancer. In certain embodiments, the single chain polypeptides are for use in treating an inflammatory condition. In certain embodiments, the single chain polypeptides are for use in treating an autoimmune disease. In certain embodiments the single chain polypeptides are for use in treating a cardiovascular disease. In certain embodiments, the single chain polypeptides are for use in treating a fibrotic disease.

The single chain polypeptide molecules described herein are contemplated for use as a medicament. Administration is effected by different ways, e.g. by intravenous, intraperitoneal, subcutaneous, intramuscular, intralesional, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently.

An “effective dose” refers to amounts of the active ingredient that are sufficient to affect the course and the severity of the disease, leading to the reduction or remission of such pathology. For example, an “effective dose” useful for treating and/or preventing a CD33⁺ cancer such as AML may be determined using known methods. Maximum tolerated doses (MTD) and maximum response doses (MRD) can be determined via established animal and human experimental protocols as well as in the examples described herein.

In some embodiments, administration of a polypeptide herein is at a dose level determined and contemplated by a medical practitioner. In certain therapeutic applications, polypeptide is administered to a patient already suffering from a cancer, in an amount sufficient to cure or at least partially arrest the symptoms of the cancer. Amounts effective for this use depend on the severity and course of the cancer, previous therapy, the patient's health status, weight, and response to the drugs, and the judgment of the treating physician. Therapeutically effective amounts are optionally determined by methods including, but not limited to, a dose escalation clinical trial, such as described in the examples.

### Methods of Identifying Cancer Targets for Therapeutic Intervention

Disclosed herein are methods of identifying a cancer target for therapeutic intervention comprising using a super-resolution microscope to image cellular features of a plurality of extracellular molecules that have been contacted by an antibody to generate a database of cellular features and selecting those extracellular molecules comprising at least one cancer target cellular feature when contacted by the antibody as a cancer target for therapeutic intervention.

### Cellular Feature

In some embodiments, wherein the cellular features comprise an image of the extracellular molecule. In some embodiments, the cellular features comprise two- or three-dimensional coordinates of the extracellular molecule. In some embodiments, the cellular features comprise intensities for the extracellular molecule. In some embodiments, the cellular features comprise all or part of a molecular fingerprint, molecular signature or map of the positions for the extracellular molecule. In some embodiments, the cellular features comprise all or part of a schematic or topical representation of an extracellular molecule on a cell or tissue. In some embodiments, the cellular features comprise proximity between two or more extracellular molecules. In some embodiments, the two or more extracellular molecules are of the same target or different targets. In some. embodiments, the cellular features comprise interactions of at least two extracellular molecules. In some embodiments, the cellular features comprise a distance or distances between two or more extracellular molecules. In some embodiments, the cellular features comprises an individual extracellular molecule or pairs of extracellular molecules. In some embodiments, the individual or pairs of extracellular molecules are adjacent or in contact with each other. In some embodiments, the individual or pairs of extracellular molecules are within a distance of about 1 nanometer of each other. In some embodiments, the individual or pairs of extracellular molecules are with a distance of about 1 nanometer to about 100 nanometers of each other. In some embodiments, the cellular features comprise individual, pairs, or triplets of extracellular molecules. In some embodiments, the individual, pairs, or triplets of extracellular molecules are adjacent or in contact with each other. In some embodiments, the individual, pairs, or triplets of extracellular molecules are within a distance of about 1 nanometer of each other. In some embodiments, the individual, pairs, or triplets of extracellular molecules are within a distance of about 1 nanometer to about 100 nanometers of each other. In some embodiments, the cellular features comprise pairs, triplets, quadruplets, quintuplets, or higher order clusters of extracellular molecules. In some embodiments, the pairs, triplets, quadruplets, quintuplets or higher order clusters or extracellular molecules are adjacent or in contact with each other. In some embodiments, the pairs, triplets, quadruplets, quintuplets or higher order clusters of extracellular molecules comprise the same or different extracellular molecules. In some embodiments, the pairs, triplets, quadruplets, quintuplets, or higher order clusters or extracellular molecules are within a distance of about 1 nanometer of each other. In some embodiments, the pairs, triplets, quadruplets, quintuplets, or higher order clusters of extracellular molecules are within a distance of about 1 nanometer to about 100 nanometers of each other. In some embodiments, the cellular features comprise a geometric relationship between two or more extracellular molecules. In some embodiments, the cellular features comprise movement of the extracellular molecule. In some embodiments, the movement is internalization or movement to an extracellular surface. In some embodiments, the cellular features comprise internalization of the extracellular molecule. In some embodiments, the cellular features comprise a change in two- or three-dimensional coordinates of the extracellular molecule. In some embodiments, the cellular features comprise a change in clustering of the extracellular molecule. In some embodiments, the cellular features comprise a change in cluster formation of the extracellular molecule. In some embodiments, the cellular features comprise movement of at least one cellular receptor. In some embodiments, the cellular features comprise an oligomerization state of an extracellular molecule. In some embodiments, the cellular features comprise a clustering state of an extracellular molecule. In some embodiments, the cellular features comprise the names of proteins clustering with an extracellular molecule. In some embodiments, the extracellular receptor comprises an extracellular receptor listed in Table 1. In some embodiments, the cellular features are present on a cell in tissue. In some embodiments, the cellular features are present on a cell in cancer or on normal tissue. In some embodiments, the cellular features are present on a normal cell, on a cancer cell or both normal and cancer cells. In some embodiments, the cellular features are present on a normal cell, a cancer cell or a cell in the tumor microenvironment. In some embodiments, the cellular features are present on a cell in the tumor microenvironment. In some embodiments, the cell in the tumor microenvironment is selected from a B cell, a T cell, a Natural Killer (NK) cell, a myeloid cell, a fibroblast, and a pericyte. In some embodiments, the cell in the tumor microenvironment is selected from a stromal cell, an epithelial cell or an adipocyte. In some embodiments, the cellular features predict the presence of a cancer cell or a cell in the tumor microenvironment. In some embodiments, the cellular features predict the presence of a normal cell. In some embodiments, the cellular features are present on the same cell in tissue. In some embodiments, the cellular features are present on at least two different cells in tissue. In some embodiments, the cellular features are within a distance or between cell distances of the at least two different cells. In some embodiments, the cellular features are present on a tissue structure in a normal or a diseased state. In some embodiments, the diseased state comprises a cancer diseased state, an immunological diseased state, a neurological diseased state, an antiviral diseased state, a cardiovascular diseased state, or an autoimmune diseased state. In some embodiments, the cellular features comprise a change in expression of the extracellular molecule. In some embodiments, the cellular features comprise a change in a glycosylation pattern of the extracellular molecule. In some embodiments, the cellular features comprise a change in enzymatic activity of an extracellular molecule. In some embodiments, the cellular features comprise a change in intracellular or intercellular communication. In some embodiments, the cellular features comprise a change in intracellular or intercellular signaling or communication. In some embodiments, the cellular features comprise a change in cell adhesion, mechanics or movement. In some embodiments, the cellular features comprise tissue localization of an extracellular receptor. In some embodiments, the tissue localization comprises a cancer structure or tumor microenvironment. In some embodiments, the cellular features comprise internalization of at least one extracellular receptor. In some embodiments, the extracellular receptor signals when contacted by the antibody. In some embodiments, different samples are assessed for common cellular features. In some embodiments, different samples are assessed for different cellular features. In some embodiments, the method further comprises characterizing the cellular features based on an intensity of the imaging agent in the image. In some embodiments, the cellular features are assessed after administration of at least one ligand. In some embodiments, the cellular features are assessed after administration of at least one therapeutic or at least one therapeutic regimen. In some embodiments, a cellular feature present in the disease-state cell and not present in the non-disease state cell is subsequently used in a development of the antibody-based therapy.

### Super-Resolution Microscope

In some embodiments, the super-resolution microscope comprises a deterministic super resolution microscope. In some embodiments, the deterministic super-resolution microscopy uses light structuring to determine the sub-diffraction limit spatial information of the sample. For example, the deterministic super-resolution microscope can use a depletion ring to refine the excitation spot of the microscope. In some embodiments, the super-resolution microscope is a stimulated emission depletion (STED) microscope, a ground state depletion (GSD) microscope saturated structed illumination microscopy (SSIM), super-resolution orthogonal deterministic imaging (SODI), or the like. In some embodiments, the super-resolution microscope is a MINFLUX microscope. For example, the super-resolution technique can be the MINFLUX technique developed by Abberior Instruments. MINFLUX is a super-resolution fluorescence microscopy method that can provide resolutions in the range of 1 to 3 nanometers (nm) by localizing individual switchable fluorophores with a probing donut-shaped excitation beam (see Gwosch et al., “MINFLUX nanoscopy delivers 3D multicolor nanometer resolution in cells”, Nature Methods, vol. 17, pp. 217-220, February 2020). In some embodiments, the super-resolution microscope uses a stochastic super-resolution technique (e.g., is a stochastic super-resolution microscope). In some embodiments, the stochastic super-resolution technique uses random chance to improve localization of emitters (e.g., by providing a flux such that only single emitters in a region are activated stochastically). In some embodiments, the super-resolution microscope is a stochastic optical reconstruction microscopy (STORM) microscope. In some embodiments, the stochastic super-resolution technique is direct stochastic optical reconstruction microscopy (dSTORM), photo-activated localization microscopy (PALM), fluorescence photo-activation localization microscopy (FPALM), spectral precision distance microscopy (SPDM), localization microscopy (e.g., single-molecule localization microscopy (SMLM)), cryogenic optical localization in three-dimensions (COLD), DNA-PAINT, or the like. In some embodiments, super-resolution microscopy or multiplexing methods as described by Jungmann et al. (U.S. Pat. No. 10,294,510B2 and EP 3027773B1) are used. In some embodiments, the imaging comprises tracking one or more ligands, therapeutics, or the cell surface receptors over a period of time.

To quantify extracellular proteins the fluorescence intensity observed can be normalized using the intrinsic molecular brightness of single fluorophore attached to the antibody. This can be done by labeling double strands of DNA with single fluorophores and attaching these to a small region of the cover slip used in imaging at a low concentration to ensure single molecules are spaced appropriately. During imaging an image is acquired of the small region using identical parameters as used for imaging cells and single molecule intensities from this region are used to calibrate the intensities acquired in the remaining image of the cells.

In some embodiments, the super-resolution microscope can be configured to take a time series of images of the sample. For example, the sample can be contacted with the antibody and imaged over time to provide a time series of the cellular feature. In this example, the time resolved data of the cellular feature provides additional data on the mechanism of action of the therapy as well as the efficacy. In some embodiments, the imaging comprises tracking the therapeutic, the cellular feature, or the cell surface receptors for a time period of at least about 0.001, 0.005, 0.01, 0.05, 0.1, 0.5, 1, 5, 10, 50, 100, 200, 300, 400, 500, 600, 700, 800, 900, 1,000, 5,000, 10,000, 50,000 or more milliseconds. In some embodiments, the imaging comprises tracking the therapeutic, the cellular feature, or the cell surface receptors for a time period of at most about 50,000, 10,000, 5,000, 1,000, 900, 800, 700, 600, 500, 400, 300, 200, 100, 50, 10, 5, 1, 0.5, 0.1, 0.05, 0.01, 0.005, 0.001, or fewer milliseconds.

### Cell/Tissue Microarray

In some embodiments, the cell comprises a fixed cell. In some embodiments, the cell is fixed in tissue. In some embodiments, the cell is in live tissue. In some embodiments, the cell comprises a live cell. In some embodiments, the cell comprises a mammalian cell. In some embodiments, the cell is derived from a tumor cell line. In some embodiments, the cell is derived from a normal cell line. In some embodiments, the cell is imaged on a tissue microarray, coverslip, flow cell, chip, or microfluidics chamber. In some embodiments, the tissue microarray, coverslip, flow cell, chip, or microfluidics chamber comprises frozen tissue. In some embodiments, the tissue microarray, coverslip, flow cell, chip, or microfluidics chamber comprises live tissue. In some embodiments, the tissue microarray, coverslip, flow cell, chip, or microfluidics chamber comprises fixed tissue. In some embodiments, the tissue microarray, coverslip, flow cell, chip, or microfluidics chamber is formed via a laser microdissection process.

### Antibody

In some embodiments, the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc. In some embodiments, the antibody is a bispecific antibody. In some embodiments, the antibody is a multispecific antibody. In some embodiments, the antibody comprises at least one, at least two, at least three, at least four, at least five, or at least six VHH domains or more. In some embodiments, the antibody comprises at least one, at least two, at least three, at least four, at least five, or at least six VL domains or more. In some embodiments, the antibody comprises at least one, at least two, at least three, at least four, at least five, or at least six VH domains or more. In some embodiments, the antibody is an immune cell engager. In some embodiments, the antibody comprises an effector domain (e.g., CD3 or CD16A). In some embodiments, the antibody-based therapy is on a CAR-T cell or myeloid cell or NK cell or other cell-based therapy. In some embodiments, the antibody is in a vaccine. In some embodiments, the antibody is a translated nucleic acid strand or strands. In some embodiments, the antibody comprises a single chain polypeptide. In some embodiments, the antibody comprises a homodimer. In some embodiments, the antibody comprises a heterodimer.

### Resolution

In some embodiments, the database of imaged cellular features are imaged at or above (e.g., at a lower) a resolution of the super-resolution microscope. For example, a super-resolution microscope with a resolution limit of 5 nanometers can image a sample at a resolution of up to 5 nanometers. In some embodiments, the super-resolution microscope has a resolution of at least about 250, 225, 200, 175, 150, 125, 100, 90, 80, 70, 60, 50, 40, 30, 20, 10, 9, 8, 7, 6, 5, 4, 3, 2, 1, or less nanometers. In some embodiments, the super-resolution microscope has a resolution of at most about 1, 2, 3, 4, 5, 6, 7, 8, 9, 10, 20, 30, 40, 50, 60, 70, 80, 90, 100, 125, 150, 175, 200, 225, 250, or more nanometers. In some embodiments, the resolution of the super-resolution microscope is a range as defined by any two of the proceeding values. For example, in some embodiments the resolution of the super-resolution microscope is 1-50 nanometers.

In some embodiments, the database of cellular features are imaged at a resolution of at least about 100 nanometers. In some embodiments, the database of cellular features are imaged at a resolution of at least about 50 nanometers. In some embodiments, the database of cellular features are imaged at a resolution of at least about 40 nanometers. In some embodiments, the database of cellular features are imaged at a resolution of at least about 30 nanometers. In some embodiments, the database of cellular features are imaged at a resolution of at least about 20 nanometers. In some embodiments, the database of cellular features are imaged at a resolution of at least about 10 nanometers. In some embodiments, the database of cellular features are imaged at a resolution of at least about 9 nanometers. In some embodiments, the database of cellular features are imaged at a resolution of at least about 8 nanometers. In some embodiments, the database of cellular features are imaged at a resolution of at least about 7 nanometers. In some embodiments, the database of cellular features are imaged at a resolution of at least about 6 nanometers. In some embodiments, the database of cellular features are imaged at a resolution of at least about 5 nanometers. In some embodiments, the database of cellular features are imaged at a resolution of at least about 4 nanometers. In some embodiments, the database of cellular features are imaged at a resolution of at least about 3 nanometers. In some embodiments, the database of cellular features are imaged at a resolution of at least about 2 nanometers. In some embodiments, the database of cellular features are imaged at a resolution of at least about 1 nanometers. In some embodiments, the database of cellular features are imaged at a single molecule resolution. In some embodiments, the database of cellular features are imaged at a single fluorophore resolution.

### High-Throughput Format

In some embodiments, the database of cellular features are imaged in a high throughput format. In some embodiments, the high throughput format comprises use of at least one 96 well plate. In some embodiments, the high throughput format comprises use of at least one 192 well plate. In some embodiments, the high throughput format comprises use of at least one 384 well plate. In some embodiments, the high throughput format comprises use of at least one 1536 well plate. In some embodiments, the high throughput format comprises the use of slides, coverslips, or flow cells containing multiple channels. In some embodiments, multiple cellular features are imaged at a substantially same time. In some embodiments, multiple cellular features are imaged sequentially. In some embodiments, multiple cellular features are imaged at substantially the same time and others are imaged sequentially. In some embodiments, the multiple cellular features are on surfaces of multiple cells. In some embodiments, imaging the cellular feature is done autonomously.

### Extracellular Molecule

In some embodiments, the extracellular molecule is a membrane-bound protein or a ligand bound to a membrane. In some embodiments, the extracellular molecule is selected from a protein ligand, a sugar, a lipid, a ligand, an extracellular receptor, a membrane-bound protein, a soluble protein, a structural protein, and a protein or ligand bound to a membrane protein. In some embodiments, the selected extracellular molecules are present in disease-state cells and are not present in non-disease state cells.

### Samples

In some embodiments, a sample can be tested to determine if the sample has cellular features that are common with a second sample derived from the same source (e.g., patient) or a different source. In some embodiments, the assessment comprises one or more of genetic testing (e.g., nucleic acid detection or sequencing), protein testing (e.g., identification of a presence or absence of a predetermined protein), size testing (e.g., determining a size distribution of the cells present in the sample), label testing (e.g., determining a presence or absence of one or more labels on the cell), or the like, or any combination thereof. In some embodiments, the sample is assessed after administration of at least one therapeutic or at least one therapeutic regimen (e.g., an antibody-based therapeutic regimen, a small molecule-based therapeutic regimen, a biologic-based regimen, etc.). For example, the sample can be contacted with a therapeutic regimen and subsequently assessed in order to determine an effect of the regimen and if the sample is of value to fully characterize. In some embodiments, the sample is assessed after administration of a natural ligand or naturally occurring agent or cofactor. In some embodiments, pre-assessing the sample can reduce the full characterization of samples that do not display a property of interest (e.g., an impact or lack thereof of the therapeutic regimen), reducing wasted time and resources.

In some embodiments, the cell comprises a live cell. For example, the cell can be gathered from a living organism and used as gathered. In another example, the cell can be from a maintained cell line. In some embodiments, the cell is a eukaryotic cell. In some embodiments, the cell comprises a mammalian cell. In some embodiments, the mammalian cell is derived from a mouse, a rat, a rabbit, a dog, a cat, a horse, a cow, a sheep, a pig, a goat, a llama, an alpaca, a yak, a camel, or a human. In some embodiments, the cell comprises a non-living cell. In certain embodiments, the non-living cell is a dead cell. In some embodiments, the cell comprises a fixed cell. In some embodiments, the fixed cell is fixed as described elsewhere herein.

In some embodiments, the disease state cells are derived from a tumor cell line. Examples of tumor cell lines include, but are not limited to 3T3 cells, A549 cells, HeLa cells, Jurkat cells, and those available from commercial suppliers of cell lines such as, for example, MilliporeSigma, ThermoFischer, and the like.

In some embodiments, the cell is imaged on a tissue microarray. For example, the cell is a part of a tissue microarray and imaged as such. In some embodiments, the tissue microarray comprises a plurality to tissue samples contained within a single object. In some embodiments, the plurality of tissue samples is a plurality of different tissue samples. In some embodiments, the use of a tissue microarray enables screening of a variety of different tissue types in shorter time than individually screening each tissue type. In some embodiments, the tissue microarray comprises one or more of frozen tissue (e.g., tissue presently or previously preserved by freezing), live tissue (e.g., currently living tissue), fixed tissue (e.g., tissue which has been fixed in a matrix or otherwise preserved (e.g., by use of aldehydes, alcohols, oxidizing agents, etc.)), or the like, or any combination thereof. In some embodiments, the tissue microarray is formed via a laser microdissection process. In some embodiments, the tissue microarray is formed via a needle-coring process. In some embodiments, the tissue microarray is formed via a microtome process.

### Therapeutic Design

In an aspect, the present disclosure provides a method of designing an antibody-based therapy comprising imaging a cell surface receptor on a cell for therapeutic intervention and characterizing a cellular feature of the imaged cell surface receptor, and identifying an antibody format based upon the characterizing of the cellular features.

In certain embodiments, disclosed herein, are antibodies useful for the treatment of a cancer or tumor. In some embodiments, the antibody-based therapy is useful for the treatment of an immunological, neurological, antiviral, cardiovascular, and/or autoimmune disease. Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will effect remission of a cancer being treated. In certain embodiments, treatment encompasses use as a prophylactic or maintenance dose intended to prevent reoccurrence or progression of a previously treated cancer or tumor. For treating bacterial or viral disease treatment includes, but is not limited to reducing one or more symptoms associated with the viral or bacterial disease, such as reducing fever, nausea, diarrhea, vomiting, sore-throat, cough, runny-nose, and/or rash. Treatment of bacterial or viral disease can reduce overall levels of virus or bacteria in the body, reduce a period which an individual can infect others, or reduce overall disease or convalescence time. Treatment of autoimmune or inflammatory diseases includes but is not limited to reduction in total or self-antibody levels, or reduction in total or self-cellular immune responses. Treatment can also be associate with specific symptoms of autoimmune disease related to an overzealous antibody or cellular immune response. Treatment of fibrotic disease may reduce or slow the appearance of fibrotic tissue or the deposition of collagen in the tissue. Treatment of cardiovascular disease may increase indicia of cardiovascular health including reducing blood pressure, reducing atherosclerotic lesions, or increasing heart function as indicated by the ability to pump blood. It is understood by those of skill in the art that not all individuals will respond equally, or at all, to a treatment that is administered, nevertheless these individuals are considered to be treated.

In certain embodiments, the single chain polypeptides are for use in treating a viral infection. In certain embodiments, the single chain polypeptides are for use in treating a bacterial infection. In certain embodiments, the single chain polypeptides are for use in treating a solid tumor cancer. In certain embodiments, the single chain polypeptides are for use in treating a hematological cancer. In certain embodiments, the single chain polypeptides are for use in treating an inflammatory condition. In certain embodiments, the single chain polypeptides are for use in treating an autoimmune disease. In certain embodiments the single chain polypeptides are for use in treating a cardiovascular disease. In certain embodiments, the single chain polypeptides are for use in treating a fibrotic disease.

The single chain polypeptide molecules described herein are contemplated for use as a medicament. Administration is effected by different ways, e.g. by intravenous, intraperitoneal, subcutaneous, intramuscular, intralesional, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently.

An “effective dose” refers to amounts of the active ingredient that are sufficient to affect the course and the severity of the disease, leading to the reduction or remission of such pathology. For example, an “effective dose” useful for treating and/or preventing a CD33″ cancer such as AML may be determined using known methods. Maximum tolerated doses (MTD) and maximum response doses (MRD) can be determined via established animal and human experimental protocols as well as in the examples described herein.

In some embodiments, administration of a polypeptide herein is at a dose level determined and contemplated by a medical practitioner. In certain therapeutic applications, polypeptide is administered to a patient already suffering from a cancer, in an amount sufficient to cure or at least partially arrest the symptoms of the cancer. Amounts effective for this use depend on the severity and course of the cancer, previous therapy, the patient's health status, weight, and response to the drugs, and the judgment of the treating physician. Therapeutically effective amounts are optionally determined by methods including, but not limited to, a dose escalation clinical trial, such as described in the examples.

Methods of Generating Databases of Cellular Features of Disease-State Cells and Non-Disease State Cells

Disclosed herein are methods of generating databases of cellular features of disease-state cells and non-disease state cells, comprising: imaging a cell selected from a disease-state cell and a non-disease state cell after contacting the cell with an antibody by super-resolution microscopy to provide an image of one or more cellular features of the cell; storing the one or more cellular features of the cell in the database; and repeating the imaging and the storing for one or more additional cells selected from disease-state cells and non-disease state cells to generate the database of cellular features of disease-state cells and non-disease state cells.

In some embodiments, the method further comprises determining differences between the one or more cellular features of a disease-state cell and a non-disease state cell stored in the database, and developing a therapeutic strategy to target the disease-state cell based on the differences between the one or more cellular features of the disease-state cell and the non-disease state cell stored in the database. In some embodiments, the disease state cell comprises a cancer disease state cell, an immunological disease state cell, a neurological disease state cell, an antiviral disease state cell, a cardiovascular disease state cell, or an autoimmune disease state cell. In some embodiments, the antibody binds to an antigen on the surface of the cell, and wherein the antigen is associated with a disease. In some embodiments, the antibody is associated with an imaging agent. In some embodiments, the antibody is directly associated with an imaging agent. In some embodiments, the antibody is indirectly associated with an imaging agent. In some embodiments, the method further comprises determining differences between the one or more cellular features of a disease-state cell and a non-disease state cell stored in the database.

### Localized K_{d}

In some embodiments, the method further comprises determining and correlating a localized affinity (K_{d}) for binding of the antibody to the one or more cellular features of the cell stored in the database, and storing the K_{d} and correlation between the K_{d} and the one or more cellular features of the cell in the database. In some embodiments, the method further comprises determining the localized K_{d} by single molecule tracking. In some embodiments, the method further comprises predicting a localized K_{d} for binding of the antibody to a second cell based on a similarity between the one or more cellular features of the cell and one or more cellular features of the second cell.

### Cellular Feature

In some embodiments, the one or more cellular features comprises an image of an extracellular molecule. In some embodiments, wherein the one or more cellular features comprises two- or three-dimensional coordinates of an extracellular molecule. In some embodiments, the one or more cellular features comprises intensities for an extracellular molecule. In some embodiments, the one or more cellular features comprises all or part of a molecular fingerprint, molecular signature or map of the positions for an extracellular molecule. In some embodiments, the one or more cellular features comprises all or part of a schematic or topical representation of an extracellular molecule on a cell or tissue. In some embodiments, the one or more cellular features comprises proximity between two or more extracellular molecules. In some embodiments, the two or more extracellular molecules are of the same target or different targets. In some embodiments, the one or more cellular features comprises interactions of at least two extracellular molecules. In some embodiments, the one or more cellular features comprises a distance or distances between two or more extracellular molecules. In some embodiments, the one or more cellular features comprise an individual extracellular molecule or pairs of extracellular molecules. In some embodiments, the individual or pairs of extracellular molecules are adjacent or in contact with each other. In some embodiments, the individual or pairs of extracellular molecules are within a distance of about 1 nanometer of each other. In some embodiments, the individual or pairs of extracellular molecules are with a distance of about 1 nanometer to about 100 nanometers of each other. In some embodiments, the one or more cellular features comprise individual, pairs, or triplets of extracellular molecules. In some embodiments, the individual, pairs, or triplets of extracellular molecules are adjacent or in contact with each other. In some embodiments, the individual, pairs, or triplets of extracellular molecules are within a distance of about 1 nanometer of each other. In some embodiments, the individual, pairs, or triplets of extracellular molecules are within a distance of about 1 nanometer to about 100 nanometers of each other. In some embodiments, the one or more cellular features comprise pairs, triplets, quadruplets, quintuplets, or higher order clusters of extracellular molecules. In some embodiments, the pairs, triplets, quadruplets, quintuplets or higher order clusters or extracellular molecules are adjacent or in contact with each other. In some embodiments, the pairs, triplets, quadruplets, quintuplets or higher order clusters of extracellular molecules comprise the same or different extracellular molecules. In some embodiments, the pairs, triplets, quadruplets, quintuplets, or higher order clusters or extracellular molecules are within a distance of about 1 nanometer of each other. In some embodiments, the pairs, triplets, quadruplets, quintuplets, or higher order clusters of extracellular molecules are within a distance of about 1 nanometer to about 100 nanometers of each other. In some embodiments, the one or more cellular features comprises a geometric relationship between two or more extracellular molecules. In some embodiments, the one or more cellular features comprises movement of at least one extracellular molecule. In some embodiments, the movement is internalization or movement to an extracellular surface. In some embodiments, the one or more cellular features comprises internalization of at least one extracellular molecule. In some embodiments, the one or more cellular features comprises a change in two- or three-dimensional coordinates of an extracellular molecule. In some embodiments, the one or more cellular features comprises a change in clustering of an extracellular molecule. In some embodiments, the one or more cellular features comprises a change in cluster formation of an extracellular molecule. In some embodiments, the one or more cellular features comprises an oligomerization state of an extracellular molecule. In some embodiments, the one or more cellular features comprises a clustering state of an extracellular molecule. In some embodiments, the one or more cellular features comprises the names of proteins clustering with an extracellular molecule. In some embodiments, an extracellular molecule is an extracellular molecule listed in Table 1. In some embodiments, the one or more cellular features is present on a cell in tissue. In some embodiments, the one or more cellular features is present on a cell in cancer or on normal tissue. In some embodiments, the one or more cellular features comprise an oligomerization state of an extracellular molecule. In some embodiments, the one or more cellular features comprise an oligomerization state of the antigen on the surface of the cell. In some embodiments, the oligomerization state is selected from a dimer, a trimer, a tetramer, a pentamer, and a higher-state oligomer. In some embodiments, the one or more cellular features comprise a clustering state of an extracellular molecule. In some embodiments, the one or more cellular features comprise a clustering state of the antigen on the surface of the cell. In some embodiments, the one or more cellular features comprise interactions between two or more extracellular molecules. In some embodiments, the one or more cellular features comprise tissue localization of an extracellular molecule. In some embodiments, the extracellular molecule is selected from an extracellular molecule listed in Table 1. In some embodiments, the one or more cellular features comprises movement of at least one cellular receptor. In some embodiments, the method further comprises characterizing the one or more cellular features based on an intensity of the imaging agent in the image.

### Antibody

In some embodiments, the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc. In some embodiments, the antibody is a bispecific antibody. In some embodiments, the antibody is a multispecific antibody. In some embodiments, the antibody comprises at least one, at least two, at least three, at least four, at least five, or at least six VHH domains or more. In some embodiments, the antibody comprises at least one, at least two, at least three, at least four, at least five, or at least six VL domains or more. In some embodiments, the antibody comprises at least one, at least two, at least three, at least four, at least five, or at least six VH domains or more. In some embodiments, the antibody is an immune cell engager. In some embodiments, the antibody comprises an effector domain (e.g., CD3 or CD16A). In some embodiments, the antibody-based therapy is on a CAR-T cell or myeloid cell or NK cell or other cell-based therapy. In some embodiments, the antibody is in a vaccine. In some embodiments, the antibody is a translated nucleic acid strand or strands. In some embodiments, the antibody comprises a single chain polypeptide. In some embodiments, the antibody comprises a homodimer. In some embodiments, the antibody comprises a heterodimer.

### Super-Resolution Microscope

In some embodiments, the super-resolution microscopy comprises stimulated emission depletion (STED) microscopy. In some embodiments, the super-resolution microscopy comprises ground state depletion (GSD) microscopy, saturated structed illumination microscopy (SSIM), super-resolution orthogonal deterministic imaging (SODI), or the like. In some embodiments, the super-resolution microscopy comprises MINFLUX microscopy. For example, the deterministic super-resolution technique can be the MINFLUX technique developed by Abberior Instruments. MINFLUX is a super-resolution fluorescence microscopy method that can provide resolutions in the range of 1 to 3 nanometers (nm) by localizing individual switchable fluorophores with a probing donut-shaped excitation beam (see Gwosch et al., “MINFLUX nanoscopy delivers 3D multicolor nanometer resolution in cells”, Nature Methods, vol. 17, pp. 217-220, February 2020). In some embodiments, the super-resolution microscope uses a stochastic super-resolution technique (e.g., is a stochastic super-resolution microscope). In some embodiments, the stochastic super-resolution technique uses random chance to improve localization of emitters (e.g., by providing a flux such that only single emitters in a region are activated stochastically). In some embodiments, the super-resolution microscopy comprises stochastic optical reconstruction microscopy (STORM) microscopy. In some embodiments, the stochastic super-resolution technique is direct stochastic optical reconstruction microscopy (dSTORM), photo-activated localization microscopy (PALM), fluorescence photo-activation localization microscopy (FPALM), spectral precision distance microscopy (SPDM), localization microscopy (e.g., single-molecule localization microscopy (SMLM)), cryogenic optical localization in three-dimensions (COLD), DNA-PAINT, or the like. In some embodiments, super-resolution microscopy or multiplexing methods as described by Jungmann et al. (U.S. Pat. No. 10,294,510B2 and EP 3027773B1) are used. In some embodiments, the imaging comprises tracking extracellular molecules of the cell over a period of time.

To quantify extracellular proteins the fluorescence intensity observed can be normalized using the intrinsic molecular brightness of single fluorophore attached to the antibody. This can be done by labeling double strands of DNA with single fluorophores and attaching these to a small region of the cover slip used in imaging at a low concentration to ensure single molecules are spaced appropriately. During imaging an image is acquired of the small region using identical parameters as used for imaging cells and single molecule intensities from this region are used to calibrate the intensities acquired in the remaining image of the cells.

In some embodiments, the super-resolution microscope can be configured to take a time series of images of the sample. For example, the sample can be contacted with the antibody-based therapy and imaged over time to provide a time series of the cellular feature. In this example, the time resolved data of the cellular feature provides additional data on the mechanism of action of the therapy as well as the efficacy. In some embodiments, the imaging comprises tracking the therapeutic, the cellular feature, or the cell surface receptors for a time period of at least about 0.001, 0.005, 0.01, 0.05, 0.1, 0.5, 1, 5, 10, 50, 100, 200, 300, 400, 500, 600, 700, 800, 900, 1,000, 5,000, 10,000, 50,000 or more milliseconds. In some embodiments, the imaging comprises tracking the therapeutic, the cellular feature, or the cell surface receptors for a time period of at most about 50,000, 10,000, 5,000, 1,000, 900, 800, 700, 600, 500, 400, 300, 200, 100, 50, 10, 5, 1, 0.5, 0.1, 0.05, 0.01, 0.005, 0.001, or fewer milliseconds.

### Cell/Tissue Microarray

In some embodiments, the cell comprises a fixed cell. In some embodiments, the cell is fixed in tissue. In some embodiments, the cell is in live tissue. In some embodiments, the cell comprises a live cell. In some embodiments, the cell comprises a mammalian cell. In some embodiments, the disease state cells are derived from a tumor cell line. In some embodiments, the normal state cells are derived from a normal cell line. In some embodiments, the cell is imaged on a tissue microarray, coverslip, flow cell, chip, or microfluidics chamber. In some embodiments, the tissue microarray, coverslip, flow cell, chip, or microfluidics chamber comprises frozen tissue. In some embodiments, the tissue microarray, coverslip, flow cell, chip, or microfluidics chamber comprises live tissue. In some embodiments, the tissue microarray, coverslip, flow cell, chip, or microfluidics chamber comprises fixed tissue.

### Resolution

In some embodiments, the imaging is performed at or above (e.g., at a lower) a resolution of the super-resolution microscope. For example, a super-resolution microscope with a resolution limit of 5 nanometers can image a sample at a resolution of up to 5 nanometers. In some embodiments, the super-resolution microscope has a resolution of at least about 250, 225, 200, 175, 150, 125, 100, 90, 80, 70, 60, 50, 40, 30, 20, 10, 9, 8, 7, 6, 5, 4, 3, 2, 1, or less nanometers. In some embodiments, the super-resolution microscope has a resolution of at most about 1, 2, 3, 4, 5, 6, 7, 8, 9, 10, 20, 30, 40, 50, 60, 70, 80, 90, 100, 125, 150, 175, 200, 225, 250, or more nanometers. In some embodiments, the resolution of the super-resolution microscope is a range as defined by any two of the proceeding values. For example, in some embodiments the resolution of the super-resolution microscope is 1-50 nanometers.

In some embodiments, the imaging is performed at a resolution of at least about 100 nanometers. In some embodiments, the imaging is performed at a resolution of at least about 50 nanometers. In some embodiments, the imaging is performed at a resolution of at least about 40 nanometers. In some embodiments, the imaging is performed at a resolution of at least about 30 nanometers. In some embodiments, the imaging is performed at a resolution of at least about 20 nanometers. In some embodiments, the imaging is performed at a resolution of at least about 10 nanometers. In some embodiments, the imaging is performed at a resolution of at least about 5 nanometers. In some embodiments, the imaging is performed at a resolution of at least about 2 nanometers. In some embodiments, the imaging is performed at a resolution of at least about 1 nanometer. In some embodiments, the imaging is performed at a single molecule resolution. In some embodiments, the imaging is performed at a single fluorophore resolution.

### High-Throughput Format

In some embodiments, the imaging is performed in a high throughput format. In some embodiments, the high throughput format comprises use of at least one 96 well plate. In some embodiments, the high throughput format comprises use of at least one 192 well plate. In some embodiments, the high throughput format comprises use of at least one 384 well plate. In some embodiments, the high throughput format comprises use of at least one 1536 well plate. In some embodiments, the high throughput format comprises the use of slides, coverslips, or flow cells containing multiple channels.

### Extracellular Molecule

In some embodiments, the extracellular molecule is a membrane-bound protein or a ligand bound to a membrane. In some embodiments, the extracellular molecule is selected from a protein ligand, a sugar, a lipid, a ligand, an extracellular receptor, a membrane-bound protein, a soluble protein, a structural protein, and a protein or ligan bound to a membrane protein.

### Samples

In some embodiments, a sample can be tested to determine if the sample has cellular features in common with a second sample derived from the same source (e.g., patient) or a different source. In some embodiments, the assessment comprises one or more of genetic testing (e.g., nucleic acid detection or sequencing), protein testing (e.g., identification of a presence or absence of a predetermined protein), size testing (e.g., determining a size distribution of the cells present in the sample), label testing (e.g., determining a presence or absence of one or more labels on the cell), or the like, or any combination thereof. In some embodiments, the sample is assessed after administration of at least one therapeutic or at least one therapeutic regimen (e.g., an antibody-based therapeutic regimen, a small molecule-based therapeutic regimen, a biologic-based regimen, etc.). For example, the sample can be contacted with a therapeutic regimen and subsequently assessed in order to determine an effect of the regimen and if the sample is of value to fully characterize. In some embodiments, the sample is assessed after administration of a natural ligand or naturally occurring agent or cofactor. In some embodiments, pre-assessing the sample can reduce the full characterization of samples that do not display a property of interest (e.g., an impact or lack thereof of the therapeutic regimen), reducing wasted time and resources.

In some embodiments, the cell comprises a live cell. For example, the cell can be gathered from a living organism and used as gathered. In another example, the cell can be from a maintained cell line. In some embodiments, the cell is a eukaryotic cell. In some embodiments, the cell comprises a mammalian cell. In some embodiments, the mammalian cell is derived from a mouse, a rat, a rabbit, a dog, a cat, a horse, a cow, a sheep, a pig, a goat, a llama, an alpaca, a yak, a camel, or a human. In some embodiments, the cell comprises a non-living cell. In certain embodiments, the non-living cell is a dead cell. In some embodiments, the cell comprises a fixed cell. In some embodiments, the fixed cell is fixed as described elsewhere herein.

In some embodiments, the disease state cells are derived from a tumor cell line. Examples of tumor cell lines include, but are not limited to 3T3 cells, A549 cells, HeLa cells, Jurkat cells, and those available from commercial suppliers of cell lines such as, for example, MilliporeSigma, ThermoFischer, and the like.

In some embodiments, the cell is imaged on a tissue microarray. For example, the cell is a part of a tissue microarray and imaged as such. In some embodiments, the tissue microarray comprises a plurality to tissue samples contained within a single object. In some embodiments, the plurality of tissue samples is a plurality of different tissue samples. In some embodiments, the use of a tissue microarray enables screening of a variety of different tissue types in shorter time than individually screening each tissue type. In some embodiments, the tissue microarray comprises one or more of frozen tissue (e.g., tissue presently or previously preserved by freezing), live tissue (e.g., currently living tissue), fixed tissue (e.g., tissue which has been fixed in a matrix or otherwise preserved (e.g., by use of aldehydes, alcohols, oxidizing agents, etc.)), or the like, or any combination thereof. In some embodiments, the tissue microarray is formed via a laser microdissection process. In some embodiments, the tissue microarray is formed via a needle-coring process. In some embodiments, the tissue microarray is formed via a microtome process.

### Methods of Characterizing Cell or Tissue Samples

Disclosed herein are methods of characterizing a cell sample or tissue sample, comprising: contacting the cell or tissue sample with an antibody, wherein the antibody binds to at least one extracellular molecule on a cell or tissue sample and is associated with an imaging agent detectable by super-resolution microscopy; imaging the cell or tissue sample by super-resolution microscopy after contacting the cell or tissue sample with the antibody to provide an image of the at least one extracellular molecule on the cell surface of the cell or tissue sample; and characterizing one or more cellular features of the cell or tissue sample based on the image of the at least one extracellular molecule on the cell or tissue sample.

In some embodiments, the method further comprises providing a multiplexed image of the cell or tissue sample by i) contacting the cell or tissue sample with a plurality of antibodies, wherein each of the plurality of antibodies are associated with an imaging agent, and ii) imaging the cell or tissue sample by super-resolution microscopy after contacting the cell or tissue sample with each of the plurality of antibodies; and wherein the method further comprises characterizing the one or more cellular features of the cell or tissue sample based on the multiplexed image.

In some embodiments, the imaging agent is a fluorophore. In some embodiments, the imaging agent is directly or indirectly coupled to a fluorophore. In some embodiments, the method further comprises characterizing the one or more cellular features based on the two- or three-dimensional coordinates of the at least one extracellular molecule or the imaging agent in the image. In some embodiments, the method further comprises characterizing the one or more cellular features based on an intensity of the imaging agent in the image. In some embodiments, the at least one cellular feature is assessed after administration of at least one ligand. In some embodiments, the at least one cellular feature is assessed after administration of at least one therapeutic or therapeutic regimen.

In some embodiments, the method further comprises determining which of the one or more cellular features are consistent on a cancer cell line for one or more indications. In some embodiments, the method further comprises determining which of the one or more cellular features are unique to a cancer cell line for one or more indications. In some embodiments, the method further comprises determining which of the one or more cellular features are consistent on non-disease state cells. In some embodiments, the method further comprises determining which of the one or more cellular features differ on cancer cells and non-disease state cells. In some embodiments, the method further comprises determining which of the one or more cellular features are the same on cancer cells and non-disease state cells. In some embodiments, the method further comprises determining which of the one or more cellular features are consistent on cancer cells. In some embodiments, the method further comprises determining which of the one or more cellular features are consistent on cancer cells among patients in one or more indications. In some embodiments, the method further comprises determining which of the one or more cellular features predict clinical toxicity. In some embodiments, the method further comprises detecting a binding mode of the antibody to the based on the image.

### Cellular Feature

In some embodiments, the one or more cellular features includes clustering of the at least one extracellular molecule. In some embodiments, the clustered extracellular molecule is functional and is involved in signaling, internalization, motion, and/or enzymatic activity. In some embodiments, the clustered extracellular molecule is nonfunctional and is aggregating or has an inability to internalize or be shed. In some embodiments, the one or more cellular features relate to a single extracellular molecule on the cell or tissue sample. In some embodiments, the one or more cellular features relate to an extracellular molecule pair on the cell or tissue sample. In some embodiments, the one or more cellular features relate to an extracellular molecule triplet, an extracellular molecule quadruplet, an extracellular molecule quintuplet, or other higher order extracellular molecule cluster on the cell or tissue sample. In some embodiments, the single extracellular molecule, the extracellular molecule pair, the extracellular molecule triplet, the extracellular molecule quadruplet, the extracellular molecule quintuplet, or the other higher order extracellular molecule cluster includes one or more other proteins. In some embodiments, the extracellular molecule pair, the extracellular molecule triplet, the extracellular molecule quadruplet, the extracellular molecule quintuplet, or other higher order extracellular molecule cluster are each composed of a single extracellular molecule or of a plurality of extracellular molecules. In some embodiments, the one or more cellular features comprise one or more cellular features of multiple extracellular molecules. In some embodiments, the one or more cellular features include a presence of the at least one extracellular molecule on the cell or tissue sample. In some embodiments, the one or more cellular features include an absence of the at least one extracellular molecule on the cell or tissue sample. In some embodiments, the one or more cellular features include an abundance of the at least one extracellular molecule on the cell or tissue sample. In some embodiments, the one or more cellular features include a distribution of the at least one extracellular molecule on the cell or tissue sample. In some embodiments, the one or more cellular features include a density of the at least one extracellular molecule on the cell or tissue sample.

In some embodiments, the one or more cellular features include an oligomeric state of the at least one extracellular molecule. In some embodiments, the one or more cellular features include a heteromeric state of the at least one extracellular molecule. In some embodiments, the one or more cellular features include one or more nearest neighbors to the at least one extracellular molecule. In some embodiments, the one or more nearest neighbors are selected from proteins, extracellular receptors, antigens, carbohydrates, and lipids. In some embodiments, the one or more cellular features include a distance between separate molecules of the single extracellular molecule. In some embodiments, the one or more cellular features include a pairwise distance between extracellular molecules of the extracellular molecule pair. In some embodiments, the one or more cellular features include a pairwise distance between extracellular molecules of the extracellular molecule triplet, the extracellular molecule quadruplet, the extracellular molecule quintuplet, or other higher order extracellular molecule cluster. In some embodiments, the one or more cellular features comprise lateral, anterior, or ventral movement of the at least one extracellular molecule.

In some embodiments, the one or more cellular features include one or more cellular features on a same cell and/or between different cells. In some embodiments, the one or more cellular features include intercellular interactions between the at least one extracellular molecule and one or more extracellular proteins on a separate cell. In some embodiments, the one or more cellular features comprise an absence of intercellular interactions between the at least one extracellular molecule and one or more extracellular proteins on a separate cell. In some embodiments, the intercellular interactions include an oligomer or a cluster of two or more extracellular proteins on separate cells. In some embodiments, the intercellular interactions are selected from tumor cell-tumor cell interactions, tumor cell-tumor microenvironment cell interactions, and tumor microenvironment cell-tumor microenvironment cell interactions. In some embodiments, the cell in the tumor microenvironment is selected from a B cell, a T cell, a Natural Killer (NK) cell, a myeloid cell, a fibroblast, and a pericyte. In some embodiments, the cell in the tumor microenvironment is selected from a stromal cell, an epithelial cell or an adipocyte.

In some embodiments, the one or more cellular features predict the presence of a cancer cell or a cell in the tumor microenvironment. In some embodiments, the one or more cellular features predict the presence of a normal cell. In some embodiments, the one or more cellular features indicate a signaling event. In some embodiments, the one or more cellular features define a molecular signature, cell signature or tissue sample signature. In some embodiments, the one or more cellular features comprises an image of the at least one extracellular molecule. In some embodiments, the one or more cellular features comprises two- or three-dimensional coordinates of the at least one extracellular molecule. In some embodiments, the one or more cellular features comprises intensities for the at least one extracellular molecule. In some embodiments, the one or more cellular features comprises all or part of a molecular fingerprint, molecular signature or map of the positions for the at least one extracellular molecule. In some embodiments, the one or more cellular features comprises all or part of a schematic or topical representation of an extracellular molecule on a cell or tissue. In some embodiments, the one or more cellular features comprises proximity between two or more extracellular molecules. In some embodiments, the two or more extracellular molecules are of the same extracellular molecule or different extracellular molecules. In some embodiments, the one or more cellular features comprises interactions of at least two extracellular molecules. In some embodiments, the one or more cellular features comprises a distance or distances between two or more extracellular molecules. In some embodiments, the one or more cellular features comprise an individual extracellular molecule or pairs of extracellular molecules. In some embodiments, the individual or pairs of extracellular molecules are adjacent or in contact with each other. In some embodiments, the individual or pairs of extracellular molecules are within a distance of about 1 nanometer of each other. In some embodiments, the individual or pairs of extracellular molecules are with a distance of about 1 nanometer to about 100 nanometers of each other. In some embodiments, the one or more cellular features comprise individual, pairs, or triplets of extracellular molecules. In some embodiments, the individual, pairs, or triplets of extracellular molecules are adjacent or in contact with each other. In some embodiments, the individual, pairs, or triplets of extracellular molecules are within a distance of about 1 nanometer of each other. In some embodiments, the individual, pairs, or triplets of extracellular molecules are within a distance of about 1 nanometer to about 100 nanometers of each other. In some embodiments, the one or more cellular features comprise pairs, triplets, quadruplets, quintuplets, or higher order clusters of extracellular molecules. In some embodiments, the pairs, triplets, quadruplets, quintuplets or higher order clusters or extracellular molecules are adjacent or in contact with each other. In some embodiments, the pairs, triplets, quadruplets, quintuplets or higher order clusters of extracellular molecules comprise the same or different extracellular molecules. In some embodiments, the pairs, triplets, quadruplets, quintuplets, or higher order clusters or extracellular molecules are within a distance of about 1 nanometer of each other. In some embodiments, the pairs, triplets, quadruplets, quintuplets, or higher order clusters of extracellular molecules are within a distance of about 1 nanometer to about 100 nanometers of each other. In some embodiments, the one or more cellular features comprises a geometric relationship between two or more extracellular molecules.

In some embodiments, the one or more cellular features comprises movement of at least one extracellular molecule. In some embodiments, the movement is internalization or movement to an extracellular surface. In some embodiments, the at least one cellular feature comprises internalization of at least one extracellular molecule. In some embodiments, the one or more cellular features comprises a change in two- or three-dimensional coordinates of an extracellular molecule. In some embodiments, the one or more cellular features comprises a change in clustering of an extracellular molecule. In some embodiments, the one or more cellular features comprises a change in cluster formation of an extracellular molecule. In some embodiments, the one or more cellular features comprises an oligomerization state of an extracellular molecule. In some embodiments, the one or more cellular features comprises a clustering state of an extracellular molecule. In some embodiments, the one or more cellular features comprises the names of proteins clustering with an extracellular molecule. In some embodiments, an extracellular molecule is an extracellular molecule listed in Table 1.

In some embodiments, the method further comprises: repeating the method for one or more additional cell or tissue samples comprising disease-state cells or non-disease state cells; and storing the one or more cellular features of each of the cell or tissue samples in a database to provide a database of cellular features of disease-state cells and non-disease state cells.

### Localized K_{d}

In some embodiments, the method further comprises correlating a localized affinity (K_{d}) for binding of the antibody to the one or more cellular features of the cell or tissue sample stored in the database, and storing the correlation between the K_{d} and the one or more cellular features of the cell or tissue sample in the database. In some embodiments, the method further comprises determining the localized K_{d} by single molecule tracking. In some embodiments, the method further comprises predicting a localized K_{d} for binding of the antibody to a second cell or tissue sample based on a similarity between the one or more cellular features of the cell or tissue sample and one or more cellular features of the second cell or tissue sample.

### Antibody

In some embodiments, the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc. In some embodiments, the antibody is a bispecific antibody. In some embodiments, the antibody is a multispecific antibody. In some embodiments, the antibody comprises at least one, at least two, at least three, at least four, at least five, or at least six VHH domains or more. In some embodiments, the antibody comprises at least one, at least two, at least three, at least four, at least five, or at least six VL domains or more. In some embodiments, the antibody comprises at least one, at least two, at least three, at least four, at least five, or at least six VH domains or more. In some embodiments, the antibody is an immune cell engager. In some embodiments, the antibody comprises an effector domain (e.g., CD3 or CD16A). In some embodiments, the antibody-based therapy is on a CAR-T cell or myeloid cell or NK cell or other cell-based therapy. In some embodiments, the antibody is in a vaccine. In some embodiments, the antibody is a translated nucleic acid strand or strands. In some embodiments, the antibody comprises a single chain polypeptide. In some embodiments, the antibody comprises a homodimer. In some embodiments, the antibody comprises a heterodimer.

### Super-Resolution Microscope

In some embodiments, the super-resolution microscope is a deterministic super-resolution microscope. In some embodiments, the deterministic super-resolution microscopy uses light structuring to determine the sub-diffraction limit spatial information of the sample. For example, the deterministic super-resolution microscope can use a depletion ring to refine the excitation spot of the microscope. In some embodiments, the super-resolution microscopy comprises stimulated emission depletion (STED) microscopy. In some embodiments, the super-resolution microscopy comprises ground state depletion (GSD) microscopy. In some embodiments, the deterministic super-resolution technique is saturated structed illumination microscopy (SSIM), super-resolution orthogonal deterministic imaging (SODI), or the like. In some embodiments, the super-resolution microscopy comprises MINFLUX microscopy. For example, the deterministic super-resolution technique can be the MINFLUX technique developed by Abberior Instruments. MINFLUX is a super-resolution fluorescence microscopy method that can provide resolutions in the range of 1 to 3 nanometers (nm) by localizing individual switchable fluorophores with a probing donut-shaped excitation beam (see Gwosch et al., “MINFLUX nanoscopy delivers 3D multicolor nanometer resolution in cells”, Nature Methods, vol. 17, pp. 217-220, February 2020). In some embodiments, the super-resolution microscope uses a stochastic super-resolution technique (e.g., is a stochastic super-resolution microscope). In some embodiments, the stochastic super-resolution technique uses random chance to improve localization of emitters (e.g., by providing a flux such that only single emitters in a region are activated stochastically). In some embodiments, the super-resolution microscopy comprises stochastic optical reconstruction microscopy (STORM) microscopy. In some embodiments, the stochastic super-resolution technique is direct stochastic optical reconstruction microscopy (dSTORM), photo-activated localization microscopy (PALM), fluorescence photo-activation localization microscopy (FPALM), spectral precision distance microscopy (SPDM), localization microscopy (e.g., single-molecule localization microscopy (SMLM)), cryogenic optical localization in three-dimensions (COLD), DNA-PAINT, or the like. In some embodiments, super-resolution microscopy or multiplexing methods as described by Jungmann et al. (U.S. Pat. No. 10,294,510B2 and EP 3027773B1) are used.

To quantify extracellular proteins the fluorescence intensity observed can be normalized using the intrinsic molecular brightness of single fluorophore attached to the antibody. This can be done by labeling double strands of DNA with single fluorophores and attaching these to a small region of the cover slip used in imaging at a low concentration to ensure single molecules are spaced appropriately. During imaging an image is acquired of the small region using identical parameters as used for imaging cells and single molecule intensities from this region are used to calibrate the intensities acquired in the remaining image of the cells.

In some embodiments, the super-resolution microscope can be configured to take a time series of images of the sample. For example, the sample can be contacted with the antibody-based therapy and imaged over time to provide a time series of the cellular feature. In this example, the time resolved data of the cellular feature provides additional data on the mechanism of action of the therapy as well as the efficacy. In some embodiments, the imaging comprises tracking the therapeutic, the cellular feature, or the cell surface receptors for a time period of at least about 0.001, 0.005, 0.01, 0.05, 0.1, 0.5, 1, 5, 10, 50, 100, 200, 300, 400, 500, 600, 700, 800, 900, 1,000, 5,000, 10,000, 50,000 or more milliseconds. In some embodiments, the imaging comprises tracking the therapeutic, the cellular feature, or the cell surface receptors for a time period of at most about 50,000, 10,000, 5,000, 1,000, 900, 800, 700, 600, 500, 400, 300, 200, 100, 50, 10, 5, 1, 0.5, 0.1, 0.05, 0.01, 0.005, 0.001, or fewer milliseconds.

### Cell/Tissue Microarray

In some embodiments, the cell or tissue sample comprises a fixed cell. In some embodiments, the cell or tissue sample comprises a live cell. In some embodiments, the cell or tissue sample comprises a mammalian cell. In some embodiments, imaging the cell or tissue sample comprises imaging the cell on a tissue microarray, coverslip, flow cell, chip, or microfluidics chamber. In some embodiments, the tissue microarray, coverslip, flow cell, chip, or microfluidics chamber, comprises frozen tissue. In some embodiments, the tissue microarray, coverslip, flow cell, chip, or microfluidics chamber, comprises live tissue. In some embodiments, the tissue microarray, coverslip, flow cell, chip, or microfluidics chamber comprises fixed tissue.

### Resolution

In some embodiments, the database of imaged cellular features are imaged at or above (e.g., at a lower) a resolution of the super-resolution microscope. For example, a super-resolution microscope with a resolution limit of 5 nanometers can image a sample at a resolution of up to 5 nanometers. In some embodiments, the super-resolution microscope has a resolution of at least about 250, 225, 200, 175, 150, 125, 100, 90, 80, 70, 60, 50, 40, 30, 20, 10, 9, 8, 7, 6, 5, 4, 3, 2, 1, or less nanometers. In some embodiments, the super-resolution microscope has a resolution of at most about 1, 2, 3, 4, 5, 6, 7, 8, 9, 10, 20, 30, 40, 50, 60, 70, 80, 90, 100, 125, 150, 175, 200, 225, 250, or more nanometers. In some embodiments, the resolution of the super-resolution microscope is a range as defined by any two of the proceeding values. For example, in some embodiments the resolution of the super-resolution microscope is 1-50 nanometers.

In some embodiments, the imaging is performed at a resolution of at least about 100 nanometers. In some embodiments, the imaging is performed at a resolution of at least about 50 nanometers. In some embodiments, the imaging is performed at a resolution of at least about 40 nanometers. In some embodiments, the imaging is performed at a resolution of at least about 30 nanometers. In some embodiments, the imaging is performed at a resolution of at least about 20 nanometers. In some embodiments, the imaging is performed at a resolution of at least about 10 nanometers. In some embodiments, the imaging is performed at a resolution of at least about 5 nanometers. In some embodiments, the imaging is performed at a resolution of at least about 2 nanometers. In some embodiments, the imaging is performed at a resolution of at least about 1 nanometer. In some embodiments, the imaging is performed at a single molecule resolution. In some embodiments, the imaging is performed at a single fluorophore resolution.

### High-Throughput Format

In some embodiments, the imaging is performed in high throughput format. In some embodiments, the high throughput format comprises use of at least one 96 well plate. In some embodiments, the high throughput format comprises use of at least one 192 well plate. In some embodiments, the high throughput format comprises use of at least one 384 well plate. In some embodiments, the high throughput format comprises use of at least one 1536 well plate. In some embodiments, the high throughput format comprises use of slides, coverslips, or flow cell containing multiple channels.

### Extracellular Molecule

In some embodiments, the at least one extracellular molecule is a membrane-bound protein or a ligand bound to a membrane. In some embodiments, the at least one extracellular molecule is selected from a protein ligand, a sugar, a lipid, a ligand, an extracellular receptor, a membrane-bound protein, a soluble protein, and a structural protein.

### Samples

In some embodiments, a sample can be tested to determine if the sample has cellular features in common with a second sample derived from the same source (e.g., patient) or a different source. In some embodiments, the assessment comprises one or more of genetic testing (e.g., nucleic acid detection or sequencing), protein testing (e.g., identification of a presence or absence of a predetermined protein), size testing (e.g., determining a size distribution of the cells present in the sample), label testing (e.g., determining a presence or absence of one or more labels on the cell), or the like, or any combination thereof. In some embodiments, the sample is assessed after administration of at least one therapeutic or at least one therapeutic regimen (e.g., an antibody-based therapeutic regimen, a small molecule-based therapeutic regimen, a biologic-based regimen, etc.). For example, the sample can be contacted with a therapeutic regimen and subsequently assessed in order to determine an effect of the regimen and if the sample is of value to fully characterize. In some embodiments, the sample is assessed after administration of a natural ligand or naturally occurring agent or cofactor. In some embodiments, pre-assessing the sample can reduce the full characterization of samples that do not display a property of interest (e.g., an impact or lack thereof of the therapeutic regimen), reducing wasted time and resources.

In some embodiments, the cell comprises a live cell. For example, the cell can be gathered from a living organism and used as gathered. In another example, the cell can be from a maintained cell line. In some embodiments, the cell is a eukaryotic cell. In some embodiments, the cell comprises a mammalian cell. In some embodiments, the mammalian cell is derived from a mouse, a rat, a rabbit, a dog, a cat, a horse, a cow, a sheep, a pig, a goat, a llama, an alpaca, a yak, a camel, or a human. In some embodiments, the cell comprises a non-living cell. In certain embodiments, the non-living cell is a dead cell. In some embodiments, the cell comprises a fixed cell. In some embodiments, the fixed cell is fixed as described elsewhere herein.

In some embodiments, the disease state cells are derived from a tumor cell line. Examples of tumor cell lines include, but are not limited to 3T3 cells, A549 cells, HeLa cells, Jurkat cells, and those available from commercial suppliers of cell lines such as, for example, MilliporeSigma, ThermoFischer, and the like.

In some embodiments, the cell is imaged on a tissue microarray. For example, the cell is a part of a tissue microarray and imaged as such. In some embodiments, the tissue microarray comprises a plurality to tissue samples contained within a single object. In some embodiments, the plurality of tissue samples is a plurality of different tissue samples. In some embodiments, the use of a tissue microarray enables screening of a variety of different tissue types in shorter time than individually screening each tissue type. In some embodiments, the tissue microarray comprises one or more of frozen tissue (e.g., tissue presently or previously preserved by freezing), live tissue (e.g., currently living tissue), fixed tissue (e.g., tissue which has been fixed in a matrix or otherwise preserved (e.g., by use of aldehydes, alcohols, oxidizing agents, etc.)), or the like, or any combination thereof. In some embodiments, the tissue microarray is formed via a laser microdissection process. In some embodiments, the tissue microarray is formed via a needle-coring process. In some embodiments, the tissue microarray is formed via a microtome process.

### Computer Systems

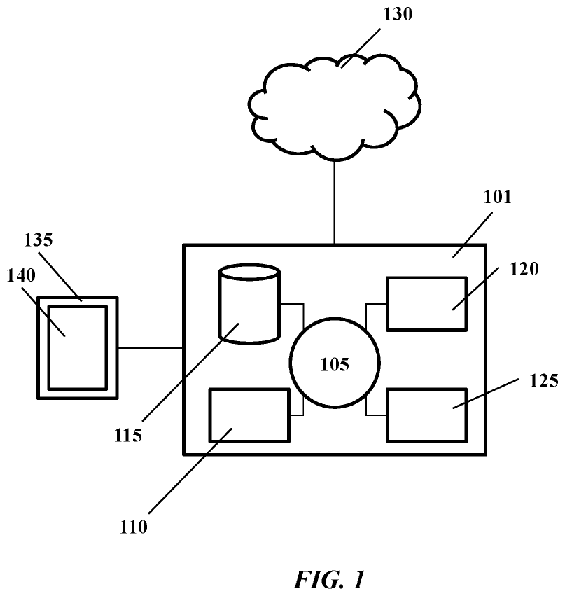

The present disclosure provides computer systems that are programmed to implement methods of the disclosure. In some embodiments, the computer systems of the present disclosure may implement scripting in ImageJ or other similar image processing program. FIG. 1 shows a computer system 101 that is programmed or otherwise configured to direct a super-resolution microscope. The computer system 101 can regulate various aspects of the present disclosure, such as, for example, operation of a microscope and analysis of the data acquired by the microscope. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 130. For example, the computer directs a cloud (e.g., remote) computing resource to execute an imaging processing program. In this example, the cloud computing resource includes one or more GPU's to improve processing speed of the images.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion. The code can comprise pre-compiled libraries. For example, the code can comprise a scripting language configured to utilize compiled libraries.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, a preview of a field of view of a super-resolution microscope. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, identify cellular features in an image.

### Embodiments

Embodiment 1 comprises a method of generating a database comprising cellular features from a disease-state cell and non-disease state cell comprising imaging a sample that has been contacted by an antibody using a super-resolution microscope and generating a database comprising at least one cellular feature of a cell in the sample and determining if the at least one cellular feature is indicative of the disease-state cell or the non-disease-state cell.

Embodiment 2 comprises the method of embodiment 1, wherein the at least one cellular feature comprises an image of an extracellular molecule.

Embodiment 3 comprises the method of embodiments 1 or 2, wherein the at least one cellular feature comprises two- or three-dimensional coordinates of an extracellular molecule.

Embodiment 4 comprises the method of any one of the preceding embodiments, wherein the at least one cellular feature comprises intensities for an extracellular molecule.

Embodiment 5 comprises the method of any one of the preceding embodiments, wherein the at least one cellular feature comprises all or part of a molecular fingerprint, molecular signature, or map of the positions for an extracellular molecule.

Embodiment 6 comprises the method of any one of the preceding embodiments, wherein the at least one cellular feature comprises all or part of a molecular fingerprint, molecular signature, or map of the positions for an extracellular molecule.

Embodiment 7 comprises the method of any one of the preceding embodiments, wherein the at least one cellular feature comprises proximity between two or more extracellular molecules.

Embodiment 8 comprises the method of embodiment 7, wherein the two or more extracellular molecules are of the same target or different targets.

Embodiment 9 comprises the method of any one of the preceding embodiments, wherein the at least one cellular feature comprises interactions of at least two extracellular molecules.

Embodiment 10 comprises the method of any one of the preceding embodiments, wherein the at least one cellular feature comprises a distance or distances between two or more extracellular molecules.

Embodiment 11 comprises the method of any one of the preceding embodiments, wherein the at least one cellular feature comprises the at least one cellular feature comprises an individual extracellular molecule or pairs of extracellular molecules.

Embodiment 12 comprises the method of embodiment 11, wherein the individual extracellular molecule or pairs of extracellular molecules are adjacent or in contact with each other.

Embodiment 13 comprises the method of embodiments 11 or 12, wherein the individual or pairs of extracellular molecules are within a distance of about 1 nanometer of each other.

Embodiment 14 comprises the method of embodiments 11 or 12, wherein the individual or pairs of extracellular molecules are with a distance of about 1 nanometer to about 100 nanometers of each other.

Embodiment 15 comprises the method of any one of the preceding embodiments, wherein the at least one cellular feature comprises an individual extracellular molecule, or pairs or triplets of extracellular molecules.

Embodiment 16 comprises the method of embodiment 15, wherein the individual extracellular molecule, pairs of extracellular molecules, or triplets of extracellular molecules are adjacent or in contact with each other.

Embodiment 17 comprises the method of embodiment 15, wherein the individual extracellular molecule, pairs of extracellular molecules, or triplets of extracellular molecules are within a distance of about 1 nanometer to about 100 nanometers of each other.

Embodiment 18 comprises the method of any one of the preceding embodiments, wherein the at least one cellular feature comprises pairs, triplets, quadruplets, quintuplets, or higher order clusters of extracellular molecules.

Embodiment 19 comprises the method of embodiment 18, wherein the pairs, triplets, quadruplets, quintuplets, or higher order clusters or extracellular molecules are adjacent or in contact with each other.

Embodiment 20 comprises the method of embodiment 18, wherein the pairs, triplets, quadruplets, quintuplets, or higher order clusters of extracellular molecules comprise the same or different molecules.

Embodiment 21 comprises the method of any one of the preceding embodiments, wherein the at least one cellular feature comprises a geometric relationship between two or more extracellular molecules.

Embodiment 22 comprises the method of any one of the preceding embodiments, wherein the at least one cellular feature comprises movement of at least one extracellular molecule.

Embodiment 23 comprises the method of embodiment 22, wherein the movement is internalization or movement to an extracellular surface.

Embodiment 24 comprises the method of any one of the preceding embodiments, wherein the at least one cellular feature comprises internalization of at least one extracellular molecule.

Embodiment 25 comprises the method of any one of the preceding embodiments, wherein the at least one cellular feature comprises a change in two- or three-dimensional coordinates of an extracellular molecule.

Embodiment 26 comprises the method of any one of the preceding embodiments, wherein the at least one cellular feature comprises a change in two- or three-dimensional coordinates of an extracellular molecule.

Embodiment 27 comprises the method of any one of the preceding embodiments, wherein the at least one cellular feature comprises a change in cluster formation of an extracellular molecule.

Embodiment 28 comprises the method of any one of the preceding embodiments, wherein the at least one cellular feature comprises an oligomerization state of an extracellular molecule.

Embodiment 29 comprises the method of any one of the preceding embodiments, wherein the at least one cellular feature comprises a clustering state of an extracellular molecule.

Embodiment 30 comprises the method of any one of the preceding embodiments, wherein the at least one cellular feature comprises the names of proteins clustering with an extracellular molecule.

Embodiment 31 comprises the method of any one of embodiments 2 to 30, wherein an extracellular molecule is an extracellular molecule listed in Table 1.

Embodiment 32 comprises the method of any one of the preceding embodiments, wherein the at least one cellular feature is present on a cell in tissue.

Embodiment 33 comprises the method of any one of embodiments 1 to 31, wherein the at least one cellular feature is present on a cell in cancer or on normal tissue.

Embodiment 34 comprises the method of any one of embodiments 1 to 31, wherein the at least one cellular feature is present on a normal cell, on a cancer cell or both normal and cancer cells.

Embodiment 35 comprises the method of any one of embodiments 1 to 31, wherein the at least one cellular feature is present on a normal cell, a cancer cell or a cell in the tumor microenvironment.

Embodiment 36 comprises the method of any one of embodiments 1 to 31, wherein the at least one cellular feature is present on a cell in the tumor microenvironment.

Embodiment 37 comprises the method of embodiments 35 or 36, wherein the cell in the tumor microenvironment is selected from a B cell, a T cell, a Natural Killer (NK) cell, a myeloid cell, a fibroblast, and a pericyte.

Embodiment 38 comprises the method of embodiments 35 or 36, wherein the cell in the tumor microenvironment is selected from a stromal cell, an epithelial cell or an adipocyte.

Embodiment 39 comprises the method of any one of the preceding embodiments, wherein the at least one cellular feature predicts the presence of a cancer cell or a cell in the tumor microenvironment.

Embodiment 40 comprises the method of any one of embodiments 1 to 38, wherein the at least one cellular feature predicts the presence of a normal cell.

Embodiment 41 comprises the method of any one of the preceding embodiments, wherein the at least one cellular feature is present on the same cell in tissue.

Embodiment 42 comprises the method of any one of embodiments 1 to 40, wherein the at least one cellular feature is present on at least two different cells in tissue.

Embodiment 43 comprises the method of embodiment 42, wherein the at least one cellular feature is within a distance or between cell distances of the at least two different cells.

Embodiment 44 comprises the method of any one of the preceding embodiments, wherein the at least one cellular feature is present on a tissue structure in a normal or a diseased state.

Embodiment 45 comprises the method of embodiment 44, wherein the diseased state comprises a cancer diseased state, an immunological diseased state, a neurological diseased state, an antiviral diseased state, a cardiovascular diseased state, or an autoimmune diseased state.

Embodiment 46 comprises the method of embodiments 44 or 45, wherein the tissue structure comprises epithelium, a duct, or a blood vessel.

Embodiment 47 comprises the method of any one of the preceding embodiments, wherein the at least one cellular feature comprises a change in expression of an extracellular molecule.

Embodiment 48 comprises the method of any one of the preceding embodiments, wherein the at least one cellular feature comprises a change in a glycosylation pattern of an extracellular molecule.

Embodiment 49 comprises the method of any one of the preceding embodiments, wherein the at least one cellular feature comprises a change in enzymatic activity of an extracellular molecule.

Embodiment 50 comprises the method of any one of the preceding embodiments, wherein the at least one cellular feature comprises a change in intracellular or intercellular communication.

Embodiment 51 comprises the method of any one of embodiments 1 to 49, wherein the at least one cellular feature comprises a change in intracellular or intercellular signaling or communication.

Embodiment 52 comprises the method of any one of the preceding embodiments, wherein the at least one cellular feature comprises a change in cell adhesion, mechanics or movement.

Embodiment 53 comprises the method of any one of the preceding embodiments, wherein the at least one cellular feature comprises tissue localization of an extracellular molecule.

Embodiment 54 comprises the method of embodiment 53, wherein the tissue localization comprises a diseased tissue or tumor microenvironment.

Embodiment 55 comprises the method of embodiment 53, wherein the tissue localization comprises a cancer structure or tumor microenvironment.

Embodiment 56 comprises the method of any one of embodiments 2 to 31, 47 to 49, and 53, wherein the extracellular molecule internalizes when contacted by the antibody.

Embodiment 57 comprises the method of any one of the preceding embodiments, wherein different samples are assessed for common cellular features.

Embodiment 58 comprises the method of any one of the preceding embodiments, wherein different samples are assessed for different cellular features.

Embodiment 59 comprises the method of any one of the preceding embodiments, further comprising characterizing the at least one cellular feature based on an intensity of the imaging agent in the image.

Embodiment 60 comprises the method of any one of the preceding embodiments, wherein the at least one cellular feature is assessed after administration of at least one ligand.

Embodiment 61 comprises the method of any one of the preceding embodiments, wherein the at least one cellular feature is assessed after administration of at least one therapeutic or at least one therapeutic regimen.

Embodiment 62 comprises the method of any one of the preceding embodiments, wherein the super-resolution microscope comprises a deterministic super resolution microscope.

Embodiment 63 comprises the method of any one of embodiments 1 to 61, wherein the super-resolution microscope is a stimulated emission depletion (STED) microscope or a ground state depletion (GSD) microscope.

Embodiment 64 comprises the method of any one of embodiments 1 to 61, wherein the super-resolution microscope comprises a stochastic super-resolution microscope.

Embodiment 65 comprises the method of any one of embodiments 1 to 61, wherein the super-resolution microscope is a MINFLUX microscope.

Embodiment 66 comprises the method of any one of embodiments 1 to 61, wherein the super-resolution microscope is a stochastic optical reconstruction microscopy (STORM) microscope.

Embodiment 67 comprises the method of any one of the preceding embodiments, wherein the imaging comprises tracking one or more ligands, therapeutics, or cell surface receptors over a period of time.

Embodiment 68 comprises the method of any one of the preceding embodiments, wherein the cell comprises a fixed cell.

Embodiment 69 comprises the method of any one of the preceding embodiments, wherein the cell is fixed in tissue.

Embodiment 70 comprises the method of any one of the preceding embodiments, wherein the cell is in live tissue.

Embodiment 71 comprises the method of any one of the preceding embodiments, wherein the cell comprises a live cell.

Embodiment 72 comprises the method of any one of the preceding embodiments, wherein the cell comprises a mammalian cell.

Embodiment 73 comprises the method of any one of the preceding embodiments, wherein the disease state cells are derived from a tumor cell line.

Embodiment 74 comprises the method of embodiment 44, wherein the normal state cells are derived from a normal cell line.

Embodiment 75 comprises the method of any one of the preceding embodiments, wherein the cell is imaged on a tissue microarray, coverslip, flow cell, chip, or microfluidics chamber.

Embodiment 76 comprises the method of embodiment 75, wherein the tissue microarray, coverslip, flow cell, chip, or microfluidics chamber comprises frozen tissue.

Embodiment 77 comprises the method of embodiment 75, wherein the tissue microarray, coverslip, flow cell, chip, or microfluidics chamber comprises live tissue.

Embodiment 78 comprises the method of embodiment 75, wherein the tissue microarray, coverslip, flow cell, chip, or microfluidics chamber comprises fixed tissue.

Embodiment 79 comprises the method of embodiment 75, wherein the tissue microarray coverslip, flow cell, chip, or microfluidics chamber is formed via a laser microdissection process.

Embodiment 80 comprises the method of any one of the preceding embodiments, wherein the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc.

Embodiment 81 comprises the method of any one of the preceding embodiments, wherein the antibody is a bispecific antibody.

Embodiment 82 comprises the method of any one of embodiments 1 to 80, wherein the antibody is a multispecific antibody.

Embodiment 83 comprises the method of any one of embodiments 1 to 82, wherein the antibody comprises at least one, at least two, at least three, at least four, at least five, or at least six VHH domains or more.

Embodiment 84 comprises the method of any one of embodiments 1 to 82, wherein the antibody comprises at least one, at least two, at least three, at least four, at least five, or at least six VL domains or more.

Embodiment 85 comprises the method of any one of embodiments 1 to 82, wherein the antibody comprises at least one, at least two, at least three, at least four, at least five, or at least six VH domains or more.

Embodiment 86 comprises the method of any one of the preceding embodiments, wherein the antibody is an immune cell engager.

Embodiment 87 comprises the method of any one of embodiments 1 to 85, wherein the antibody comprises an effector domain (e.g., CD3 or CD16A).

Embodiment 88 comprises the method of any one of embodiments 1 to 85, wherein the antibody-based therapy is on a CAR-T cell or myeloid cell or NK cell or other cell-based therapy.

Embodiment 89 comprises the method of any one of the preceding embodiments, wherein the antibody is in a vaccine.

Embodiment 90 comprises the method of any one of the preceding embodiments, wherein the antibody is a translated nucleic acid strand or strands.

Embodiment 91 comprises the method of any one of embodiments 1 to 89, wherein the antibody comprises a single chain polypeptide.

Embodiment 92 comprises the method of any one of the preceding embodiments, wherein the antibody comprises a homodimer.

Embodiment 93 comprises the method of any one of embodiments 1 to 91, wherein the antibody comprises a heterodimer.

Embodiment 94 comprises the method of any one of the preceding embodiments, wherein the database of imaged cellular features are imaged at a resolution of at least about 50 nanometers.

Embodiment 95 comprises the method of any one of embodiments 1 to 93, wherein the database of imaged cellular features are imaged at a resolution of at least about 40 nanometers.

Embodiment 96 comprises the method of any one of embodiments 1 to 93, wherein the database of imaged cellular features are imaged at a resolution of at least about 30 nanometers.

Embodiment 97 comprises the method of any one of embodiments 1 to 93, wherein the database of imaged cellular features are imaged at a resolution of at least about 20 nanometers.

Embodiment 98 comprises the method of any one of embodiments 1 to 93, wherein the database of imaged cellular features are imaged at a resolution of at least about 10 nanometers.

Embodiment 99 comprises the method of any one of embodiments 1 to 93, wherein the database of imaged cellular features are imaged at a resolution of at least about 9 nanometers.

Embodiment 100 comprises the method of any one of embodiments 1 to 93, wherein the database of imaged cellular features are imaged at a resolution of at least about 8 nanometers.

Embodiment 101 comprises the method of any one of embodiments 1 to 93, wherein the database of imaged cellular features are imaged at a resolution of at least about 7 nanometers.

Embodiment 102 comprises the method of any one of embodiments 1 to 93, wherein the database of imaged cellular features are imaged at a resolution of at least about 6 nanometers.

Embodiment 103 comprises the method of any one of embodiments 1 to 93, wherein the database of imaged cellular features are imaged at a resolution of at least about 5 nanometers.

Embodiment 104 comprises the method of any one of embodiments 1 to 93, wherein the database of imaged cellular features are imaged at a resolution of at least about 4 nanometers.

Embodiment 105 comprises the method of any one of embodiments 1 to 93, wherein the database of imaged cellular features are imaged at a resolution of at least about 3 nanometers.

Embodiment 106 comprises the method of any one of embodiments 1 to 93, wherein the database of imaged cellular features are imaged at a resolution of at least about 2 nanometers.

Embodiment 107 comprises the method of any one of embodiments 1 to 93, wherein the database of imaged cellular features are imaged at a resolution of at least about 1 nanometers.

Embodiment 108 comprises the method of any one of embodiments 1 to 93, wherein the database of imaged cellular features are imaged at a single molecule resolution.

Embodiment 109 comprises the method of any one of embodiments 1 to 93, wherein the database of imaged cellular features are imaged at a single fluorophore resolution.

Embodiment 110 comprises the method of any one of the preceding embodiments, wherein the database of imaged cellular features are imaged in a high throughput format.

Embodiment 111 comprises the method of embodiment 110, wherein the high throughput format comprises use of at least one 96 well plate.

Embodiment 112 comprises the method of embodiment 110, wherein the high throughput format comprises use of at least one 192 well plate.

Embodiment 113 comprises the method of embodiment 110, wherein the high throughput format comprises use of at least one 384 well plate.

Embodiment 114 comprises the method of embodiment 110, wherein the high throughput format comprises use of at least one 1536 well plate.

Embodiment 115 comprises the method of embodiment 110, wherein the high throughput format comprises the use of slides, coverslips, or flow cells containing multiple channels.

Embodiment 116 comprises the method of any one of the preceding embodiments, wherein multiple cellular features are imaged at substantially the same time.

Embodiment 117 comprises the method of any one of embodiments 1 to 115, wherein multiple cellular features are imaged sequentially.

Embodiment 118 comprises the method of any one of embodiments 1 to 115, wherein multiple cellular features are images at substantially the same time and others are imaged sequentially.

Embodiment 119 comprises the method of any one of embodiments 116 to 118, wherein the multiple cellular features are on surfaces of multiple cells.

Embodiment 120 comprises the method of any one of the preceding embodiments, wherein imaging of a cellular feature is completed in less than about 5 minutes.

Embodiment 121 comprises the method of any one of the preceding embodiments, wherein imaging the database of imaged cellular features is done autonomously.

Embodiment 122 comprises the method of any one of embodiments 2 to 121, wherein the extracellular molecule is a membrane-bound protein or a ligand bound to a membrane.

Embodiment 123 comprises the method of any one of embodiments 2 to 121, wherein the extracellular molecule is selected from a protein ligand, a sugar, a lipid, a ligand, an extracellular receptor, a membrane-bound protein, a soluble protein, a structural protein, and a protein or ligand bound to a membrane protein.

Embodiment 124 comprises a method of designing an antibody-based therapy comprising imaging an extracellular molecule on a cell for therapeutic intervention using a super-resolution microscope and characterizing a cellular feature of the imaged extracellular molecule and designing an antibody format based upon the characterizing of the cellular feature.

Embodiment 125 comprises the method of embodiment 124, wherein the cellular feature comprises an image of the extracellular molecule.

Embodiment 126 comprises the method of embodiments 124 or 125, wherein the cellular feature comprises two- or three-dimensional coordinates of the extracellular molecule.

Embodiment 127 comprises the method of any one of embodiments 124 to 126, wherein the cellular feature comprises intensities for the extracellular molecule.

Embodiment 128 comprises the method of any one of embodiments 124 to 127, wherein the cellular feature comprises all or part of a molecular fingerprint, molecular signature, or map of the positions for the extracellular molecule.

Embodiment 129 comprises the method of any one of embodiments 124 to 128, wherein the cellular feature comprises all or part of a schematic or topical representation of an extracellular molecule on a cell or tissue.

Embodiment 130 comprises the method of any one of embodiments 124 to 129, wherein the cellular feature comprises proximity between two or more extracellular molecules.

Embodiment 131 comprises the method of embodiment 130, wherein the two or more extracellular molecules are of the same target or different targets.

Embodiment 132 comprises the method of any one of embodiments 124 to 131, wherein the cellular feature comprises interactions of at least two extracellular molecules.

Embodiment 133 comprises the method of any one of embodiments 124 to 132, wherein the cellular feature comprises a distance or distances between two or more extracellular molecules.

Embodiment 134 comprises the method of any one of embodiments 124 to 133, wherein the cellular feature comprises an individual extracellular molecule or pairs of extracellular molecules.

Embodiment 135 comprises the method of embodiment 134, wherein the individual extracellular molecule or pairs of extracellular molecules are adjacent or in contact with each other.

Embodiment 136 comprises the method of embodiments 134 or 135, wherein the individual extracellular molecule or pairs of extracellular molecules are within a distance of about 1 nanometer of each other.

Embodiment 137 comprises the method of embodiments 134 or 135, wherein the individual extracellular molecule or pairs of extracellular molecules are with a distance of about 1 nanometer to about 100 nanometers of each other.

Embodiment 138 comprises the method of any one of embodiments 124 to 133, wherein the cellular feature comprises an individual extracellular molecule, pairs of extracellular molecules, or triplets of extracellular molecules.

Embodiment 139 comprises the method of embodiment 138, wherein the individual extracellular molecule, pairs of extracellular molecules, or triplets of extracellular molecules are adjacent or in contact with each other.

Embodiment 140 comprises the method of embodiment 138, wherein the individual extracellular molecule, pairs of extracellular molecules, or triplets of extracellular molecules are within a distance of about 1 nanometer to about 100 nanometers of each other.

Embodiment 141 comprises the method of any one of embodiments 124 to 133, wherein the cellular feature comprises pairs, triplets, quadruplets, quintuplets, or higher order clusters of extracellular molecules.

Embodiment 142 comprises the method of embodiment 141, wherein the pairs, triplets, quadruplets, quintuplets, or higher order clusters or extracellular molecules are adjacent or in contact with each other.

Embodiment 143 comprises the method of embodiment 141, wherein the pairs, triplets, quadruplets, quintuplets, or higher order clusters of extracellular molecules comprise the same or different extracellular molecules.

Embodiment 144 comprises the method of any one of embodiments 124 to 143, wherein the cellular feature comprises a geometric relationship between two or more extracellular molecules.

Embodiment 145 comprises the method of any one of embodiments 124 to 144, wherein the cellular feature comprises movement of the extracellular molecule.

Embodiment 146 comprises the method of embodiment 145, wherein the movement is internalization or movement to an extracellular surface.

Embodiment 147 comprises the method of any one of embodiments 124 to 146, wherein the cellular feature comprises internalization of the extracellular molecule.

Embodiment 148 comprises the method of any one of embodiments 124 to 147, wherein the cellular feature comprises a change in two- or three-dimensional coordinates of the extracellular molecule.

Embodiment 149 comprises the method of any one of embodiments 124 to 148, herein the cellular feature comprises a change in clustering of the extracellular molecule

Embodiment 150 comprises the method of any one of embodiments 124 to 149, wherein the cellular feature comprises a change in cluster formation of the extracellular molecule.

Embodiment 151 comprises the method of any one of embodiments 124 to 150, wherein the cellular feature comprises movement of at least one cellular receptor.

Embodiment 152 comprises the method of any one of embodiments 124 to 151, wherein the cellular feature comprises an oligomerization state of an extracellular molecule.

Embodiment 153 comprises the method of any one of embodiments 124 to 152, wherein the cellular feature comprises a clustering state of an extracellular molecule.

Embodiment 154 comprises the method of any one of embodiments 124 to 153, wherein the cellular feature comprises the names of proteins clustering with an extracellular molecule.

Embodiment 155 comprises the method of any one of embodiments 124 to 154, wherein the extracellular molecule comprises an extracellular molecule listed in Table 1.

Embodiment 156 comprises the method of any one of embodiments 124 to 155, wherein the cellular feature is present on a cell in tissue.

Embodiment 157 comprises the method of any one of embodiments 124 to 156, wherein the cellular feature is present on a cell in cancer or on normal tissue.

Embodiment 158 comprises the method of any one of embodiments 124 to 156, wherein the cellular feature is present on a normal cell, on a cancer cell or both normal and cancer cells.

Embodiment 159 comprises the method of any one of embodiments 124 to 156, wherein the cellular feature is present on a normal cell, a cancer cell or a cell in the tumor microenvironment.

Embodiment 160 comprises the method of any one of embodiments 124 to 156, wherein the cellular feature is present on a cell in the tumor microenvironment.

Embodiment 161 comprises the method of embodiments 159 or 160, wherein the cell in the tumor microenvironment is selected from a B cell, a T cell, a Natural Killer (NK) cell, a myeloid cell, a fibroblast, and a pericyte.

Embodiment 162 comprises the method of embodiments 159 or 160, wherein the cell in the tumor microenvironment is selected from a stromal cell, an epithelial cell or an adipocyte.

Embodiment 163 comprises the method of any one of embodiments 124 to 162, wherein the cellular feature predicts the presence of a cancer cell or a cell in the tumor microenvironment.

Embodiment 164 comprises the method of any one of embodiments 124 to 162, wherein the cellular feature predicts the presence of a normal cell.

Embodiment 165 comprises the method of any one of embodiments 124 to 164, wherein the cellular feature is present on the same cell in tissue.

Embodiment 166 comprises the method of any one of embodiments 124 to 164, wherein the cellular feature is present on at least two different cells in tissue.

Embodiment 167 comprises the method of embodiment 166, wherein the cellular feature is within a distance or between cell distances of the at least two different cells.

Embodiment 168 comprises the method of any one of embodiments 124 to 167, wherein the cellular feature is present on a tissue structure in a normal or a diseased state.

Embodiment 169 comprises the method of embodiment 168, wherein the diseased state comprises a cancer diseased state, an immunological diseased state, a neurological diseased state, an antiviral diseased state, a cardiovascular diseased state, or an autoimmune diseased state.

Embodiment 170 comprises the method of any one of embodiments 124 to 169, wherein the cellular feature comprises a change in expression of the extracellular molecule.

Embodiment 171 comprises the method of any one of embodiments 124 to 170, wherein the cellular feature comprises a change in a glycosylation pattern of the extracellular molecule.

Embodiment 172 comprises the method of any one of embodiments 124 to 171, wherein the cellular feature comprises a change in enzymatic activity of an extracellular molecule.

Embodiment 173 comprises the method of any one of embodiments 124 to 172, wherein the cellular feature comprises a change in intracellular or intercellular communication.

Embodiment 174 comprises the method of any one of embodiments 124 to 173, wherein the cellular feature comprises a change in intracellular or intercellular signaling or communication.

Embodiment 175 comprises the method of any one of embodiments 124 to 174, wherein the cellular feature comprises a change in cell adhesion, mechanics or movement.

Embodiment 176 comprises the method of any one of embodiments 124 to 175, wherein the cellular feature comprises tissue localization of the extracellular molecule.

Embodiment 177 comprises the method of embodiment 176, wherein the tissue localization comprises a diseased tissue structure or microenvironment.

Embodiment 178 comprises the method of embodiment 176, wherein the tissue localization comprises a cancer structure or tumor microenvironment.

Embodiment 179 comprises the method of any one of embodiments 124 to 178, wherein the extracellular molecule signals when contacted by the antibody-based therapy.

Embodiment 180 comprises the method of any one of embodiments 124 to 179, wherein different samples are assessed for common cellular features.

Embodiment 181 comprises the method of any one of embodiments 124 to 180, wherein different samples are assessed for different cellular features.

Embodiment 182 comprises the method any one of embodiments 124 to 181, further comprising characterizing the cellular feature based on an intensity of the imaging agent in the image,

Embodiment 183 comprises the method of any one of embodiments 124 to 182, wherein the cellular feature is assessed after administration of at least one ligand.

Embodiment 184 comprises the method of any one of embodiments 124 to 183, wherein the cellular feature is assessed after administration of at least one therapeutic or at least one therapeutic regimen.

Embodiment 185 comprises the method of any one of embodiments 124 to 184, wherein the super-resolution microscope comprises a deterministic super resolution microscope.

Embodiment 186 comprises the method of any one of embodiments 124 to 184, wherein the super-resolution microscope is a stimulated emission depletion (STED) microscope or a ground state depletion (GSD) microscope.

Embodiment 187 comprises the method of any one of embodiments 124 to 184, wherein the super-resolution microscope comprises a stochastic super-resolution microscope.

Embodiment 188 comprises the method of any one of embodiments 124 to 184, wherein the super-resolution microscope is a MINFLUX microscope.

Embodiment 189 comprises the method of any one of embodiments 124 to 184, wherein the super-resolution microscope is a stochastic optical reconstruction microscopy (STORM) microscope.

Embodiment 190 comprises the method of any one of embodiments 124 to 189, wherein the imaging comprises tracking the antibody-based therapy or the cell surface receptor over a period of time.

Embodiment 191 comprises the method of any one of embodiments 124 to 190, wherein the cell comprises a fixed cell.

Embodiment 192 comprises the method of any one of embodiments 124 to 191, wherein the cell is fixed in tissue.

Embodiment 193 comprises the method of any one of embodiments 124 to 190, wherein the cell is in live tissue.

Embodiment 194 comprises the method of any one of embodiments 124 to 190, wherein the cell comprises a live cell.

Embodiment 195 comprises the method of any one of embodiments 124 to 194, wherein the cell comprises a mammalian cell.

Embodiment 196 comprises the method of any one of embodiments 124 to 195, wherein the cell is imaged on a tissue microarray, coverslip, flow cell, chip, or microfluidics chamber.

Embodiment 197 comprises the method of embodiment 196, wherein the tissue microarray, coverslip, flow cell, chip, or microfluidics chamber comprises frozen tissue.

Embodiment 198 comprises the method of embodiment 196, wherein the tissue microarray, coverslip, flow cell, chip, or microfluidics chamber comprises live tissue.

Embodiment 199 comprises the method of embodiment 196, wherein the tissue microarray, coverslip, flow cell, chip, or microfluidics chamber comprises fixed tissue.

Embodiment 200 comprises the method of embodiment 196, wherein the tissue microarray, coverslip, flow cell, chip, or microfluidics chamber is formed via a laser microdissection process.

Embodiment 201 comprises the method of any one of embodiments 124 to 200, wherein the antibody-based therapy comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc.

Embodiment 202 comprises the method of any one of embodiments 124 to 201, wherein the antibody-based therapy is a bispecific antibody.

Embodiment 203 comprises the method of any one of embodiments 124 to 201, wherein the antibody-based therapy is a multispecific antibody.

Embodiment 204 comprises the method of any one of embodiments 124 to 203, wherein the antibody-based therapy comprises at least one, at least two, at least three, at least four, at least five, or at least six VHH domains or more.

Embodiment 205 comprises the method of any one of embodiments 124 to 203, wherein the antibody-based therapy comprises at least one, at least two, at least three, at least four, at least five, or at least six VL domains or more.

Embodiment 206 comprises the method of any one of embodiments 124 to 203, wherein the antibody-based therapy comprises at least one, at least two, at least three, at least four, at least five, or at least six VH domains or more.

Embodiment 207 comprises the method of any one of embodiments 124 to 206, wherein the antibody-based therapy is an immune cell engager.

Embodiment 208 comprises the method of any one of embodiments 124 to 206, wherein the antibody-based therapy comprises an effector domain (e.g., CD3 or CD16A).

Embodiment 209 comprises the method of any one of embodiments 124 206, wherein the antibody-based therapy is on a CAR-T cell or myeloid cell or NK cell or other cell-based therapy.

Embodiment 210 comprises the method of any one of embodiments 124 to 209, wherein the antibody-based therapy is in a vaccine.

Embodiment 211 comprises the method of any one of embodiments 124 to 210, wherein the antibody-based therapy is a translated nucleic acid strand or strands.

Embodiment 212 comprises the method of any one of embodiments 124 to 210, wherein the antibody-based therapy comprises a single chain polypeptide.

Embodiment 213 comprises the method of any one of embodiments 124 to 212, wherein the antibody-based therapy comprises a homodimer.

Embodiment 214 comprises the method of any one of embodiments 124 to 212, wherein the antibody-based therapy comprises a heterodimer.

Embodiment 215 comprises the method of any one of embodiments 124 to 214, wherein the cellular feature is imaged at a resolution of at least about 50 nanometers.

Embodiment 216 comprises the method of any one of embodiments 124 to 214, wherein the cellular feature is imaged at a resolution of at least about 40 nanometers.

Embodiment 217 comprises the method of any one of embodiments 124 to 214, wherein the cellular feature is imaged at a resolution of at least about 30 nanometers.

Embodiment 218 comprises the method of any one of embodiments 124 to 214, wherein the cellular feature is imaged at a resolution of at least about 20 nanometers.

Embodiment 219 comprises the method of any one of embodiments 124 to 214, wherein the cellular feature is imaged at a resolution of at least about 10 nanometers.

Embodiment 220 comprises the method of any one of embodiments 124 to 214, wherein the cellular feature is imaged at a resolution of at least about 9 nanometers.

Embodiment 221 comprises the method of any one of embodiments 124 to 214, wherein the cellular feature is imaged at a resolution of at least about 8 nanometers.

Embodiment 222 comprises the method of any one of embodiments 124 to 214, wherein the cellular feature is imaged at a resolution of at least about 7 nanometers.

Embodiment 223 comprises the method of any one of embodiments 124 to 214, wherein the cellular feature is imaged at a resolution of at least about 6 nanometers.

Embodiment 224 comprises the method of any one of embodiments 124 to 214, wherein the cellular feature is imaged at a resolution of at least about 5 nanometers.

Embodiment 225 comprises the method of any one of embodiments 124 to 214, wherein the cellular feature is imaged at a resolution of at least about 4 nanometers.

Embodiment 226 comprises the method of any one of embodiments 124 to 214, wherein the cellular feature is imaged at a resolution of at least about 3 nanometers.

Embodiment 227 comprises the method of any one of embodiments 124 to 214, wherein the cellular feature is imaged at a resolution of at least about 2 nanometers.

Embodiment 228 comprises the method of any one of embodiments 124 to 214, wherein the cellular feature is imaged at a resolution of at least about 1 nanometers.

Embodiment 229 comprises the method of any one of embodiments 124 to 214, wherein the cellular feature is imaged at a single molecule resolution.

Embodiment 230 comprises the method of any one of embodiments 124 to 214, wherein the cellular feature is imaged at a single fluorophore resolution.

Embodiment 231 comprises the method of any one of embodiments 124 to 230, wherein the cellular feature is imaged in a high throughput format.

Embodiment 232 comprises the method of embodiment 231, wherein the high throughput format comprises use of at least one 96 well plate.

Embodiment 233 comprises the method of embodiment 231, wherein the high throughput format comprises use of at least one 192 well plate.

Embodiment 234 comprises the method of embodiment 231, wherein the high throughput format comprises use of at least one 384 well plate.

Embodiment 235 comprises the method of embodiment 231, wherein the high throughput format comprises use of at least one 1536 well plate.

Embodiment 236 comprises the method of embodiment 231, wherein the high throughput format comprises the use of slides, coverslips, or flow cells containing multiple channels.

Embodiment 237 comprises the method of any one of embodiments 124 to 236, wherein multiple cellular features are imaged at substantially the same time.

Embodiment 238 comprises the method of any one of embodiments 124 to 236, wherein multiple cellular features are imaged sequentially.

Embodiment 239 comprises the method of any one of embodiments 124 to 236, wherein multiple cellular features are imaged at substantially the same time and others are imaged sequentially.

Embodiment 240 comprises the method of any one of embodiments 237 to 239, wherein the multiple cellular features are on surfaces of multiple cells.

Embodiment 241 comprises the method of any one of embodiments 124 to 240, wherein imaging of a cellular feature is completed in less than about 5 minutes.

Embodiment 242 comprises the method of any one of embodiments 124 to 241, wherein the imaging the cellular feature is done autonomously.

Embodiment 243 comprises the method of any one of embodiments 124 to 242, wherein the extracellular molecule is a membrane-bound protein or a ligand bound to a membrane.

Embodiment 244 comprises the method of any one of embodiments 124 to 242, wherein the extracellular molecule is selected from a protein ligand, a sugar, a lipid, a ligand, an extracellular receptor, a membrane-bound protein, a soluble protein, a structural protein, and a protein or ligan bound to a membrane protein.

Embodiment 245 comprises a method of identifying a cancer target for therapeutic intervention comprising using a super-resolution microscope to image cellular features of a plurality of extracellular molecules that have been contacted by an antibody to generate a database of cellular features and selecting those extracellular molecules comprising at least one cancer target cellular feature when contacted by the antibody as a cancer target for therapeutic intervention.

Embodiment 246 comprises the method of embodiment 245, wherein the cellular features comprise an image of the extracellular molecule.

Embodiment 247 comprises the method of embodiments 245 or 246, wherein the cellular features comprise two- or three-dimensional coordinates of the extracellular molecule.

Embodiment 248 comprises the method of any one of embodiments 245 to 247, wherein the cellular features comprise intensities for the extracellular molecule.

Embodiment 249 comprises the method of any one of embodiments 245 to 248, wherein the cellular features comprise all or part of a molecular fingerprint, molecular signature or map of the positions for the extracellular molecule.

Embodiment 250 comprises the method of any one of embodiments 245 to 249, wherein the cellular features comprise all or part of a schematic or topical representation of an extracellular molecule on a cell or tissue.

Embodiment 251 comprises the method of any one of embodiments 245 to 250, wherein the cellular features comprise proximity between two or more extracellular molecules.

Embodiment 252 comprises the method of embodiment 251, wherein the two or more extracellular molecules are of the same target or different targets.

Embodiment 253 comprises the method of any one of embodiments 245 to 252, wherein the cellular features comprise interactions of at least two extracellular molecules.

Embodiment 254 comprises the method of any one of embodiments 245 to 253, wherein the cellular features comprise a distance or distances between two or more extracellular molecules.

Embodiment 255 comprises the method of any one embodiments 245 to 254, wherein the cellular features comprise an individual extracellular molecule or pairs of extracellular molecules.

Embodiment 256 comprises the method of embodiment 255, wherein the individual or pairs of extracellular molecules are adjacent or in contact with each other.

Embodiment 257 comprises the method of embodiments 255 or 256, wherein the individual or pairs of extracellular molecules are within a distance of about 1 nanometer of each other.

Embodiment 258 comprises the method of embodiments 255 or 256, wherein the individual or pairs of extracellular molecules are with a distance of about 1 nanometer to about 100 nanometers of each other.

Embodiment 259 comprises the method of any one of embodiments 245 to 258, wherein the cellular features comprise an individual extracellular molecule or pairs, or triplets of extracellular molecules.

Embodiment 260 comprises the method of embodiment 259, wherein the individual, pairs, or triplets of extracellular molecules are adjacent or in contact with each other.

Embodiment 261 comprises the method of embodiment 259, wherein the individual, pairs, or triplets of extracellular molecules are within a distance of about 1 nanometer to about 100 nanometers of each other.

Embodiment 262 comprises the method of any one of embodiments 245 to 261, wherein the cellular features comprise pairs, triplets, quadruplets, quintuplets, or higher order clusters of extracellular molecules.

Embodiment 263 comprises the method of embodiment 262, wherein the pairs, triplets, quadruplets, quintuplets, or higher order clusters or extracellular molecules are adjacent or in contact with each other.

Embodiment 264 comprises the method of embodiment 262, wherein the pairs, triplets, quadruplets, quintuplets, or higher order clusters of extracellular molecules comprise the same or different molecules.

Embodiment 265 comprises the method of any one of embodiments 245 to 264, wherein the cellular features comprise a geometric relationship between two or more extracellular molecules.

Embodiment 266 comprises the method of any one of embodiments 245 to 265, wherein the cellular features comprise movement of the extracellular molecule.

Embodiment 267 comprises the method of embodiment 266, wherein the movement is internalization or movement to an extracellular surface.

Embodiment 268 comprises the method of any one of embodiments 245 to 267, wherein the cellular features comprise internalization of the extracellular molecule.

Embodiment 269 comprises the method of any one of embodiments 245 to 268, wherein the cellular features comprise a change in two- or three-dimensional coordinates of the extracellular molecule.

Embodiment 270 comprises the method of any one of embodiments 245 to 269, wherein the cellular features comprise a change in clustering of the extracellular molecule.

Embodiment 271 comprises the method of any one of embodiments 245 to 270, wherein the cellular features comprise a change in cluster formation of the extracellular molecule.

Embodiment 272 comprises the method of any one of embodiments 245 to 271, wherein the cellular features comprise movement of at least one cellular receptor.

Embodiment 273 comprises the method of any one of embodiments 245 to 272, wherein the cellular features comprise an oligomerization state of an extracellular molecule.

Embodiment 274 comprises the method of any one of embodiments 245 to 273, wherein the cellular features comprise a clustering state of an extracellular molecule.

Embodiment 275 comprises the method of any one of embodiments 245 to 274, wherein the cellular features comprise the names of proteins clustering with an extracellular molecule.

Embodiment 276 comprises the method of any one of embodiments 245 to 275, wherein the extracellular receptor comprises an extracellular receptor listed in Table 1.

Embodiment 277 comprises the method of any one of embodiments 245 to 276, wherein the cellular features are present on a cell in tissue.

Embodiment 278 comprises the method of any one of embodiments 245 to 277, wherein the cellular features are present on a cell in cancer or on normal tissue.

Embodiment 279 comprises the method of any one of embodiments 245 to 278, wherein the cellular features are present on a normal cell, on a cancer cell or both normal and cancer cells.

Embodiment 280 comprises the method of any one of embodiments 245 to 279, wherein the cellular features are present on a normal cell, a cancer cell or a cell in the tumor microenvironment.

Embodiment 281 comprises the method of any one of embodiments 245 to 280, wherein the cellular features are present on a normal cell, a cancer cell or a cell in the tumor microenvironment.

Embodiment 282 comprises the method of embodiments 280 or 281, wherein the cell in the tumor microenvironment is selected from a B cell, a T cell, a Natural Killer (NK) cell, a myeloid cell, a fibroblast, and a pericyte.

Embodiment 283 comprises the method of embodiments 280 or 281, wherein the cell in the tumor microenvironment is selected from a stromal cell, an epithelial cell or an adipocyte.

Embodiment 284 comprises the method of any one of embodiments 245 to 283, wherein the cellular features predict the presence of a cancer cell or a cell in the tumor microenvironment.

Embodiment 285 comprises the method of any one of embodiments 245 to 283, wherein the cellular features predict the presence of a normal cell.

Embodiment 286 comprises the method of any one of embodiments 245 to 285, wherein the cellular features are present on the same cell in tissue.

Embodiment 287 comprises the method of any one of embodiments 245 to 285, wherein the cellular features are present on at least two different cells in tissue.

Embodiment 288 comprises the method of any one of embodiments 245 to 287, wherein the cellular features are within a distance or between cell distances of the at least two different cells.

Embodiment 289 comprises the method of any one of embodiments 245 to 288, wherein the cellular features are present on a tissue structure in a normal or a diseased state,

Embodiment 290 comprises the method of embodiment 289, wherein the diseased state comprises a cancer diseased state, an immunological diseased state, a neurological diseased state, an antiviral diseased state, a cardiovascular diseased state, or an autoimmune diseased state.

Embodiment 291 comprises the method of any one of embodiments 245 to 290, wherein the cellular features comprise a change in expression of the extracellular molecule.

Embodiment 292 comprises the method of any one of embodiments 245 to 291, wherein the cellular features comprise a change in a glycosylation pattern of the extracellular molecule.

Embodiment 293 comprises the method of any one of embodiments 245 to 292, wherein the cellular features comprise a change in enzymatic activity of an extracellular molecule.

Embodiment 294 comprises the method of any one of embodiments 245 to 293, wherein the cellular features comprise a change in intracellular or intercellular communication.

Embodiment 295 comprises the method of any one of embodiments 245 to 293, wherein the cellular features comprise a change in intracellular or intercellular signaling or communication.

Embodiment 296 comprises the method of any one of embodiments 245 to 295, wherein the cellular features comprise a change in cell adhesion, mechanics or movement.

Embodiment 297 comprises the method of any one of embodiments 245 to 296, wherein the cellular features comprise tissue localization of an extracellular receptor.

Embodiment 298 comprises the method of embodiment 297, wherein the tissue localization comprises a cancer structure or tumor microenvironment.

Embodiment 299 comprises the method of any one of embodiments 245 to 298, wherein the cellular features comprise internalization of at least one extracellular receptor.

Embodiment 300 comprises the method of any one of embodiments 245 to 299, wherein the extracellular receptor signals when contacted by the antibody.

Embodiment 301 comprises the method of any one of embodiments 245 to 300, wherein different samples are assessed for common cellular features.

Embodiment 302 comprises the method of any one of embodiments 245 to 301, wherein different samples are assessed for different cellular features.

Embodiment 303 comprises the method of any one of embodiments 245 to 302, further comprising characterizing the cellular features based on an intensity of the imaging agent in the image.

Embodiment 304 comprises the method of any one of embodiments 245 to 303, wherein the cellular features are assessed after administration of at least one ligand.

Embodiment 305 comprises the method of any one of embodiments 245 to 304, wherein the cellular features are assessed after administration of at least one therapeutic or at least one therapeutic regimen.

Embodiment 306 comprises the method of any one of embodiments 245 to 305, wherein the super-resolution microscope comprises a deterministic super resolution microscope.

Embodiment 307 comprises the method of any one of embodiments 245 to 305, wherein the super-resolution microscope is a stimulated emission depletion (STED) microscope or a ground state depletion (GSD) microscope.

Embodiment 308 comprises the method of embodiment 307, wherein the STED microscope is a MINFLUX microscope.

Embodiment 309 comprises the method of any one of embodiments 245 to 305, wherein the super-resolution microscope comprises a stochastic super-resolution microscope.

Embodiment 310 comprises the method of any one of embodiments 245 to 305, wherein the super-resolution microscope is a stochastic optical reconstruction microscopy (STORM) microscope.

Embodiment 311 comprises the method of any one of embodiments 245 to 310, wherein the imaging comprises tracking one or more ligands, therapeutics, or the cell surface receptors over a period of time.

Embodiment 312 comprises the method of any one of embodiments 245 to 311, wherein the cell comprises a fixed cell.

Embodiment 313 comprises the method of any one of embodiments 245 to 311, wherein the cell is fixed in tissue.

Embodiment 314 comprises the method of any one of embodiments 245 to 311, wherein the cell is in live tissue.

Embodiment 315 comprises the method of any one of embodiments 245 to 311, wherein the cell comprises a live cell.

Embodiment 316 comprises the method of any one of embodiments 245 to 315, wherein the cell comprises a mammalian cell.

Embodiment 317 comprises the method of any one of embodiments 245 to 316, wherein the cell is derived from a tumor cell line.

Embodiment 318 comprises the method of any one of embodiments 245 to 316, wherein the cell is derived from a normal cell line.

Embodiment 319 comprises the method of any one of embodiments 245 to 318, wherein the cell is imaged on a tissue microarray, coverslip, flow cell, chip, or microfluidics chamber.

Embodiment 320 comprises the method of embodiment 319, wherein the tissue microarray, coverslip, flow cell, chip, or microfluidics chamber comprises frozen tissue.

Embodiment 321 comprises the method of embodiment 319, wherein the tissue microarray, coverslip, flow cell, chip, or microfluidics chamber comprises live tissue.

Embodiment 322 comprises the method of embodiment 319, wherein the tissue microarray, coverslip, flow cell, chip, or microfluidics chamber comprises fixed tissue.

Embodiment 323 comprises the method of embodiment 319, wherein the tissue microarray, coverslip, flow cell, chip, or microfluidics chamber is formed via a laser microdissection process.

Embodiment 324 comprises the method of any one of embodiments 245 to 323, wherein the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc.

Embodiment 325 comprises the method of any one of embodiments 245 to 324, wherein the antibody is a bispecific antibody.

Embodiment 326 comprises the method of any one of embodiments 245 to 324, wherein the antibody is a multispecific antibody.

Embodiment 327 comprises the method of any one of embodiments 245 to 326, wherein the antibody comprises at least one, at least two, at least three, at least four, at least five, or at least six VHH domains or more.

Embodiment 328 comprises the method of any one of embodiments 245 to 326, wherein the antibody comprises at least one, at least two, at least three, at least four, at least five, or at least six VL domains or more.

Embodiment 329 comprises the method of any one of embodiments 245 to 326, wherein the antibody comprises at least one, at least two, at least three, at least four, at least five, or at least six VH domains or more.

Embodiment 330 comprises the method of any one of embodiments 245 to 329, wherein the antibody is an immune cell engager.

Embodiment 331 comprises the method of any one of embodiments 245 to 329, wherein the antibody comprises an effector domain (e.g., CD3 or CD16A).

Embodiment 332 comprises the method of any one of embodiments 245 to 329, wherein the antibody is on a CAR-T cell or myeloid cell or NK cell or other cell-based therapy.

Embodiment 333 comprises the method of any one of embodiments 245 to 329, wherein the antibody is in a vaccine.

Embodiment 334 comprises the method of any one of embodiments 245 to 333, wherein the antibody is a translated nucleic acid strand or strands.

Embodiment 335 comprises the method of any one of embodiments 245 to 334, wherein the antibody comprises a single chain polypeptide.

Embodiment 336 comprises the method of any one of embodiments 245 to 335, wherein the antibody comprises a homodimer.

Embodiment 337 comprises the method of any one of embodiments 245 to 335, wherein the antibody comprises a heterodimer.

Embodiment 338 comprises the method of any one of embodiments 245 to 337, wherein the database of cellular features are imaged at a resolution of at least about 50 nanometers.

Embodiment 339 comprises the method of any one of embodiments 245 to 337, wherein the database of cellular features are imaged at a resolution of at least about 40 nanometers.

Embodiment 340 comprises the method of any one of embodiments 245 to 337, wherein the database of cellular features are imaged at a resolution of at least about 30 nanometers.

Embodiment 341 comprises the method of any one of embodiments 245 to 337, wherein the database of cellular features are imaged at a resolution of at least about 20 nanometers.

Embodiment 342 comprises the method of any one of embodiments 245 to 337, wherein the database of cellular features are imaged at a resolution of at least about 10 nanometers.

Embodiment 343 comprises the method of any one of embodiments 245 to 337, wherein the database of cellular features are imaged at a resolution of at least about 9 nanometers.

Embodiment 344 comprises the method of any one of embodiments 245 to 337, wherein the database of cellular features are imaged at a resolution of at least about 8 nanometers.

Embodiment 345 comprises the method of any one of embodiments 245 to 337, wherein the database of cellular features are imaged at a resolution of at least about 7 nanometers.

Embodiment 346 comprises the method of any one of embodiments 245 to 337, wherein the database of cellular features are imaged at a resolution of at least about 6 nanometers.

Embodiment 347 comprises the method of any one of embodiments 245 to 337, wherein the database of cellular features are imaged at a resolution of at least about 5 nanometers.

Embodiment 348 comprises the method of any one of embodiments 245 to 337, wherein the database of cellular features are imaged at a resolution of at least about 4 nanometers.

Embodiment 349 comprises the method of any one of embodiments 245 to 337, wherein the database of cellular features are imaged at a resolution of at least about 3 nanometers.

Embodiment 350 comprises the method of any one of embodiments 245 to 337, wherein the database of cellular features are imaged at a resolution of at least about 2 nanometers.

Embodiment 351 comprises the method of any one of embodiments 245 to 337, wherein the database of cellular features are imaged at a resolution of at least about 1 nanometers.

Embodiment 352 comprises the method of any one of embodiments 245 to 337, wherein the database of cellular features are imaged at a single molecule resolution.

Embodiment 353 comprises the method of any one of embodiments 245 to 337, wherein the database of cellular features are imaged at a single fluorophore resolution.

Embodiment 354 comprises the method of any one of embodiments 245 to 353, wherein the database of cellular features are imaged in a high throughput format.

Embodiment 355 comprises the method of embodiment 354, wherein the high throughput format comprises use of at least one 96 well plate.

Embodiment 356 comprises the method of embodiment 354, wherein the high throughput format comprises use of at least one 192 well plate.

Embodiment 357 comprises the method of embodiment 354, wherein the high throughput format comprises use of at least one 384 well plate.

Embodiment 358 comprises the method of embodiment 354, wherein the high throughput format comprises use of at least one 1536 well plate.

Embodiment 359 comprises the method of embodiment 354, wherein the high throughput format comprises the use of slides, coverslips, or flow cells containing multiple channels.

Embodiment 360 comprises the method of any one of embodiments 245 to 359, wherein multiple cellular features are imaged at a substantially same time.

Embodiment 361 comprises the method of any one of embodiments 245 to 359, wherein multiple cellular features are imaged sequentially.

Embodiment 362 comprises the method of any one of embodiments 245 to 359, wherein multiple cellular features are imaged at substantially the same time and others are imaged sequentially.

Embodiment 363 comprises the method of any one of embodiments 360 to 362, wherein the multiple cellular features are on surfaces of multiple cells.

Embodiment 364 comprises the method of any one of embodiments 245 to 363, wherein imaging the cellular feature is done autonomously.

Embodiment 365 comprises the method of any one of embodiments 245 to 364, wherein the extracellular molecule is a membrane-bound protein or a ligand bound to a membrane.

Embodiment 366 comprises the method of any one of embodiments 245 to 364, wherein the extracellular molecule is selected from a protein ligand, a sugar, a lipid, a ligand, an extracellular receptor, a membrane-bound protein, a soluble protein, a structural protein, and a protein or ligand bound to a membrane protein.

Embodiment 367 comprises the method of any one of embodiments 245 to 366, wherein the selected extracellular molecules are present in disease-state cells and are not present in non-disease state cells.

Embodiment 368 comprises the method of any one of embodiments 245 to 366, wherein a cellular feature present in the disease-state cell and not present in the non-disease state cell is subsequently used in a development of the antibody-based therapy.

Embodiment 369 comprises a method of generating a database of cellular features of disease-state cells and non-disease state cells comprising: imaging a cell selected from a disease-state cell and a non-disease state cell after contacting the cell with an antibody by super-resolution microscopy to provide an image of one or more cellular features of the cell; storing the one or more cellular features of the cell in the database; and repeating the imaging and the storing for one or more additional cells selected from disease-state cells and non-disease state cells to generate the database of cellular features of disease-state cells and non-disease state cells.

Embodiment 370 comprises the method of embodiment 369, wherein the disease state cell comprises a cancer disease state cell, an immunological disease state cell, a neurological disease state cell, an antiviral disease state cell, a cardiovascular disease state cell, or an autoimmune disease state cell.

Embodiment 371 comprises the method of embodiments 369 or 370, wherein the antibody binds to an antigen on the surface of the cell, and wherein the antigen is associated with a disease.

Embodiment 372 comprises the method of any one of embodiments 369 to 371, wherein the antibody is associated with an imaging agent.

Embodiment 373 comprises the method of any one of embodiments 369 to 372, wherein the antibody is directly associated with an imaging agent.

Embodiment 374 comprises the method of any one of embodiments 369 to 372, wherein the antibody is indirectly associated with an imaging agent.

Embodiment 375 comprises the method of any one of embodiments 369 to 374, further comprising: determining differences between the one or more cellular features of a disease-state cell and a non-disease state cell stored in the database; and developing a therapeutic strategy to target the disease-state cell based on the differences between the one or more cellular features of the disease-state cell and the non-disease state cell stored in the database.

Embodiment 376 comprises the method of any one of embodiments 369 to 375, further comprising determining and correlating a localized affinity (K_{d}) for binding of the antibody to the one or more cellular features of the cell stored in the database, and storing the K_{d} and correlation between the K_{d} and the one or more cellular features of the cell in the database.

Embodiment 377 comprises the method of embodiment 376, further comprising determining the localized K_{d} by single molecule tracking.

Embodiment 378 comprises the method of embodiments 376 or 377, further comprising predicting a localized K_{d} for binding of the antibody to a second cell based on a similarity between the one or more cellular features of the cell and one or more cellular features of the second cell.

Embodiment 379 comprises the method of any one of embodiments 369 to 378, wherein the one or more cellular features comprises an image of an extracellular molecule.

Embodiment 380 comprises the method of any one of embodiments 369 to 379, wherein the one or more cellular features comprises two- or three-dimensional coordinates of an extracellular molecule.

Embodiment 381 comprises the method of any one of embodiments 369 to 380, wherein the one or more cellular features comprises intensities for an extracellular molecule.

Embodiment 382 comprises the method of any one of embodiments 369 to 381, wherein the one or more cellular features comprises all or part of a molecular fingerprint, molecular signature or map of the positions for an extracellular molecule.

Embodiment 383 comprises the method of any one of embodiments 369 to 382, wherein the one or more cellular features comprises all or part of a schematic or topical representation of an extracellular molecule on a cell or tissue.

Embodiment 384 comprises the method of any one of embodiments 369 to 382, wherein the one or more cellular features comprises proximity between two or more extracellular molecules.

Embodiment 385 comprises the method of embodiment 384, wherein the two or more extracellular molecules are of the same target or different targets.

Embodiment 386 comprises the method of any one of embodiments 369 to 385, wherein the one or more cellular features comprises interactions of at least two extracellular molecules.

Embodiment 387 comprises the method of any one of embodiments 369 to 386, wherein the one or more cellular features comprises a distance or distances between two or more extracellular molecules.

Embodiment 388 comprises the method of any one of embodiments 369 to 387, wherein the one or more cellular features comprises an individual extracellular molecule or pairs of extracellular molecules.

Embodiment 389 comprises the method of embodiment 388, wherein the individual extracellular molecule or pairs of extracellular molecules are adjacent or in contact with each other.

Embodiment 390 comprises the method of embodiments 388 or 389, wherein the individual extracellular molecule or pairs of extracellular molecules are within a distance of about 1 nanometer of each other.

Embodiment 391 comprises the method of embodiment 388 or 389, wherein the individual or pairs of extracellular molecules are with a distance of about 1 nanometer to about 100 nanometers of each other.

Embodiment 392 comprises the method of any one of embodiments 369 to 391, wherein the one or more cellular features comprises individual extracellular molecules or pairs or triplets of extracellular molecules.

Embodiment 393 comprises the method of embodiment 392, wherein the individual extracellular molecules or pairs or triplets of extracellular molecules are adjacent or in contact with each other.

Embodiment 394 comprises the method of embodiment 392, wherein the individual extracellular molecules or pairs or triplets of extracellular molecules are within a distance of about 1 nanometer to about 100 nanometers of each other.

Embodiment 395 comprises the method of any one of embodiments 369 to 394, wherein the one or more cellular features comprises pairs, triplets, quadruplets, quintuplets, or higher order clusters of extracellular molecules.

Embodiment 396 comprises the method of embodiment 395, wherein the pairs, triplets, quadruplets, quintuplets, or higher order clusters or extracellular molecules are adjacent or in contact with each other.

Embodiment 397 comprises the method of embodiment 395, wherein the pairs, triplets, quadruplets, quintuplets, or higher order clusters of extracellular molecules comprise the same or different extracellular molecules.

Embodiment 398 comprises the method of any one of embodiments 369 to 397, wherein the one or more cellular features comprises a geometric relationship between two or more extracellular molecules.

Embodiment 399 comprises the method of any one of embodiments 369 to 398, wherein the one or more cellular features comprises movement of at least one extracellular molecule.

Embodiment 400 comprises the method embodiment 399, wherein the movement is internalization or movement to an extracellular surface.

Embodiment 401 comprises the method of any one of embodiments 369 to 400, wherein the one or more cellular features comprises internalization of at least one extracellular molecule.

Embodiment 402 comprises the method of any one of embodiments 369 to 401, wherein the one or more cellular features comprises a change in two- or three-dimensional coordinates of an extracellular molecule.

Embodiment 403 comprises the method of any one of embodiments 369 to 402, wherein the one or more cellular features comprises a change in clustering of an extracellular molecule.

Embodiment 404 comprises the method of any of any one of embodiments 369 to 403, wherein the one or more cellular features comprises a change in cluster formation of an extracellular molecule.

Embodiment 405 comprises the method of any one of embodiments 369 to 404, wherein the one or more cellular features comprises an oligomerization state of an extracellular molecule.

Embodiment 406 comprises the method of any one of embodiments 369 to 405, wherein the one or more cellular features comprises a clustering state of an extracellular molecule.

Embodiment 407 comprises the method of any one of embodiments 369 to 406, wherein the one or more cellular features comprises the names of proteins clustering with an extracellular molecule.

Embodiment 408 comprises the method of any one of embodiments 379 to 407, wherein an extracellular molecule is an extracellular molecule listed in Table 1.

Embodiment 409 comprises the method of any one of embodiments 369 to 408, wherein the one or more cellular features is present on a cell in tissue.

Embodiment 410 comprises the method of any one of embodiments 369 to 409, wherein the one or more cellular features is present on a cell in cancer or on normal tissue.

Embodiment 411 comprises the method of any one of embodiments 369 to 410, wherein the one or more cellular features comprise an oligomerization state of an extracellular molecule.

Embodiment 412 comprises the method of any one of embodiments 371 to 411, wherein the one or more cellular features comprise an oligomerization state of the antigen on the surface of the cell.

Embodiment 413 comprises the method of embodiments 411 or 412, wherein the oligomerization state is selected from a dimer, a trimer, a tetramer, a pentamer, and a higher-state oligomer.

Embodiment 414 comprises the method of any one of embodiments 369 to 413, wherein the one or more cellular features comprise interactions between two or more extracellular molecules.

Embodiment 415 comprises the method of any one of embodiments 369 to 414, wherein the one or more cellular features comprise tissue localization of an extracellular molecule.

Embodiment 416 comprises the method of embodiment 415, wherein the tissue localization comprises a diseased tissue or tumor microenvironment.

Embodiment 417 comprises the method of embodiment 415, wherein the tissue localization comprises a cancer structure or tumor microenvironment.

Embodiment 418 comprises the method of any one of embodiments 369 to 417, wherein the one or more cellular features comprises movement of at least one cellular receptor.

Embodiment 419 comprises the method of any one of embodiments 372 to 418, further comprising characterizing the one or more cellular features based on an intensity of the imaging agent in the image.

Embodiment 420 comprises the method of any one of embodiments 369 to 419, wherein the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc.

Embodiment 421 comprises the method of any one of embodiments 369 to 420, wherein the antibody is a bispecific antibody.

Embodiment 422 comprises the method of any one of embodiments 369 to 420, wherein the antibody is a multispecific antibody.

Embodiment 423 comprises the method of any one of embodiments 369 to 422, wherein the antibody comprises at least one, at least two, at least three, at least four, at least five, or at least six VHH domains or more.

Embodiment 424 comprises the method of any one of embodiments 369 to 422, wherein the antibody comprises at least one, at least two, at least three, at least four, at least five, or at least six VL domains or more.

Embodiment 425 comprises the method of any one of embodiments 369 to 422, wherein the antibody comprises at least one, at least two, at least three, at least four, at least five, or at least six VH domains or more.

Embodiment 426 comprises the method of any one of embodiments 369 to 425, wherein the antibody is an immune cell engager.

Embodiment 427 comprises the method of any one of embodiments 369 to 425, wherein the antibody comprises an effector domain (e.g., CD3 or CD16A).

Embodiment 428 comprises the method of any one of embodiments 369 to 425, wherein the antibody is on a CAR-T cell or myeloid cell or NK cell or other cell-based therapy.

Embodiment 429 comprises the method of any one of embodiments 369 to 428, wherein the antibody is in a vaccine.

Embodiment 430 comprises the method of any one of embodiments 369 to 429, wherein the antibody is a translated nucleic acid strand or strands.

Embodiment 431 comprises the method of any one of embodiments 369 to 430, wherein the antibody comprises a single chain polypeptide.

Embodiment 432 comprises the method of any one of embodiments 369 to 431, wherein the antibody comprises a homodimer.

Embodiment 433 comprises the method of any one of embodiments 369 to 432, wherein the antibody comprises a heterodimer.

Embodiment 434 comprises the method of any one of embodiments 369 to 433, wherein the super-resolution microscopy comprises stimulated emission depletion (STED) microscopy.

Embodiment 435 comprises the method of any one of embodiments 369 to 433, wherein the super-resolution microscopy comprises ground state depletion (GSD) microscopy.

Embodiment 436 comprises the method of any one of embodiments 369 to 433, wherein the super-resolution microscopy comprises stochastic optical reconstruction microscopy (STORM) microscopy.

Embodiment 437 comprises the method of any one of embodiments 369 to 433, herein the super-resolution microscopy comprises MINFLUX microscopy.

Embodiment 438 comprises the method of any one of embodiments 369 to 437, wherein the imaging comprises tracking extracellular molecules of the cell over a period of time.

Embodiment 439 comprises the method of any one of embodiments 369 to 438, wherein the cell comprises a fixed cell.

Embodiment 440 comprises the method of any one of embodiments 369 to 438, wherein the cell is fixed in tissue.

Embodiment 441 comprises the method of any one of embodiments 369 to 438, wherein the cell is in live tissue.

Embodiment 442 comprises the method of any one of embodiments 369 to 438, wherein the cell comprises a live cell.

Embodiment 443 comprises the method of any one of embodiments 369 to 442, wherein the cell comprises a mammalian cell.

Embodiment 444 comprises the method of any one of embodiments 369 to 443, wherein the disease state cells are derived from a tumor cell line.

Embodiment 445 comprises the method of any one of embodiments 369 to 444, wherein the non-disease state cells are derived from a normal cell line.

Embodiment 446 comprises the method of any one of embodiments 369 to 445, wherein the cell is imaged on a tissue microarray, coverslip, flow cell, chip, or microfluidics chamber.

Embodiment 447 comprises the method of embodiment 446, wherein the tissue microarray, coverslip, flow cell, chip, or microfluidics chamber comprises frozen tissue.

Embodiment 448 comprises the method of embodiment 446, wherein the tissue microarray, coverslip, flow cell, chip, or microfluidics chamber comprises live tissue.

Embodiment 449 comprises the method of embodiment 446, wherein the tissue microarray, coverslip, flow cell, chip, or microfluidics chamber comprises fixed tissue.

Embodiment 450 comprises the method of any one of embodiments 369 to 449, wherein the imaging is performed at a resolution of at least about 50 nanometers.

Embodiment 451 comprises the method of any one of embodiments 369 to 449, wherein the imaging is performed at a resolution of at least about 40 nanometers.

Embodiment 452 comprises the method of any one of embodiments 369 to 449, wherein the imaging is performed at a resolution of at least about 30 nanometers.

Embodiment 453 comprises the method of any one of embodiments 369 to 449, wherein the imaging is performed at a resolution of at least about 20 nanometers.

Embodiment 454 comprises the method of any one of embodiments 369 to 449, wherein the imaging is performed at a resolution of at least about 10 nanometers.

Embodiment 455 comprises the method of any one of embodiments 369 to 449, wherein the imaging is performed at a resolution of at least about 5 nanometers.

Embodiment 456 comprises the method of any one of embodiments 369 to 449, wherein the imaging is performed at a resolution of at least about 2 nanometers.

Embodiment 457 comprises the method of any one of embodiments 369 to 449, wherein the imaging is performed at a resolution of at least about 1 nanometers.

Embodiment 458 comprises the method of any one of embodiments 369 to 449, wherein the imaging is performed at a single molecule resolution.

Embodiment 459 comprises the method of any one of embodiments 369 to 449, wherein the imaging is performed at a single fluorophore resolution.

Embodiment 460 comprises the method of any one of embodiments 369 to 459, wherein the imaging is performed in a high throughput format.

Embodiment 461 comprises the method of embodiment 460, wherein the high throughput format comprises use of at least one 96 well plate.

Embodiment 462 comprises the method of embodiment 460, wherein the high throughput format comprises use of at least one 192 well plate.

Embodiment 463 comprises the method of embodiment 460, wherein the high throughput format comprises use of at least one 384 well plate.

Embodiment 464 comprises the method of embodiment 460, wherein the high throughput format comprises use of at least one 1536 well plate.

Embodiment 465 comprises the method of embodiment 460, wherein the high throughput format comprises the use of slides, coverslips, or flow cells containing multiple channels.

Embodiment 466 comprises the method of any one of embodiments 379 to 465, wherein the extracellular molecule is a membrane-bound protein or a ligand bound to a membrane.

Embodiment 467 comprises the method of any one of embodiments 379 to 465, wherein the extracellular molecule is selected from a protein ligand, a sugar, a lipid, a ligand, an extracellular receptor, a membrane-bound protein, a soluble protein, a structural protein, and a protein or ligand bound to a membrane protein.

Embodiment 468 comprises a method of characterizing a cell sample or tissue sample, comprising: contacting the cell or tissue sample with an antibody, wherein the antibody binds to at least one extracellular molecule on a cell or tissue sample and is associated with an imaging agent detectable by super-resolution microscopy; imaging the cell or tissue sample by super-resolution microscopy after contacting the cell or tissue sample with the antibody to provide an image of the at least one extracellular molecule on the cell surface of the cell or tissue sample; and characterizing one or more cellular features of the cell or tissue sample based on the image of the at least one extracellular molecule on the cell or tissue sample.

Embodiment 469 comprises the method of embodiment 468, wherein the method further comprises providing a multiplexed image of the cell or tissue sample by i) contacting the cell or tissue sample with a plurality of antibodies, wherein each of the plurality of antibodies are associated with an imaging agent, and ii) imaging the cell or tissue sample by super-resolution microscopy after contacting the cell or tissue sample with each of the plurality of antibodies; and wherein the method further comprises characterizing the one or more cellular features of the cell or tissue sample based on the multiplexed image.

Embodiment 470 comprises the method of embodiments 468 or 469, wherein the imaging agent is a fluorophore.

Embodiment 471 comprises the method of any one of embodiments 468 to 470, wherein the imaging agent is directly or indirectly coupled to a fluorophore.

Embodiment 472 comprises the method of any one of embodiments 468 to 471, further comprising characterizing the one or more cellular features based on the two- or three-dimensional coordinates of the at least one extracellular molecule or the imaging agent in the image.

Embodiment 473 comprises the method of any one of embodiments 468 to 472, further comprising characterizing the one or more cellular features based on an intensity of the imaging agent in the image.

Embodiment 474 comprises the method of any one of embodiments 468 to 473, wherein the one or more cellular features are assessed after administration of at least one ligand.

Embodiment 475 comprises the method of any one of embodiments 468 to 474, wherein the at least one cellular feature is assessed after administration of at least one therapeutic or therapeutic regimen.

Embodiment 476 comprises the method of any one of embodiments 468 to 475, wherein the one or more cellular features includes clustering of the at least one extracellular molecule.

Embodiment 477 comprises the method of embodiment 476, wherein the clustered extracellular molecule is functional and is involved in signaling, internalization, motion, and/or enzymatic activity.

Embodiment 478 comprises the method of embodiment 476, wherein the clustered extracellular molecule is nonfunctional and is aggregating or has an inability to internalize or be shed.

Embodiment 479 comprises the method of any one of embodiments 468 to 478, wherein the one or more cellular features relate to a single extracellular molecule on the cell or tissue sample.

Embodiment 480 comprises the method of any one of embodiments 468 to 478, wherein the one or more cellular features relate to an extracellular molecule pair on the cell or tissue sample.

Embodiment 481 comprises the method of any one of embodiments 468 to 478, wherein the one or more cellular features relate to an extracellular molecule triplet, an extracellular molecule quadruplet, an extracellular molecule quintuplet, or other higher order extracellular molecule cluster on the cell or tissue sample.

Embodiment 482 comprises the method of any one of embodiments 479 to 481, wherein the single extracellular molecule, the extracellular molecule pair, the extracellular molecule triplet, the extracellular molecule quadruplet, the extracellular molecule quintuplet, or the other higher order extracellular molecule cluster includes one or more other proteins.

Embodiment 483 comprises the method of any one of embodiments 480 to 482, wherein the extracellular molecule pair, the extracellular molecule triplet, the extracellular molecule quadruplet, the extracellular molecule quintuplet, or other higher order extracellular molecule cluster are each composed of a single extracellular molecule or of a plurality of extracellular molecules.

Embodiment 484 comprises the method of any one of embodiments 468 to 483, wherein the one or more cellular features comprise one or more cellular features of multiple extracellular molecules.

Embodiment 485 comprises the method of any one of embodiments 468 to 484, wherein the one or more cellular features include a presence of the at least one extracellular molecule on the cell or tissue sample.

Embodiments 486 comprises the method of any one of embodiments 468 to 484, wherein the one or more cellular features include an absence of the at least one extracellular molecule on the cell or tissue sample.

Embodiment 487 comprises the method of any one of embodiments 468 to 486, wherein the one or more cellular features include an abundance of the at least one extracellular molecule on the cell or tissue sample.

Embodiment 488 comprises the method of any one of embodiments 468 to 487, wherein the one or more cellular features include a distribution of the at least one extracellular molecule on the cell or tissue sample.

Embodiment 489 comprises the method of any one of embodiments 468 to 488, wherein the one or more cellular features include a density of the at least one extracellular molecule on the cell or tissue sample.

Embodiment 490 comprises the method of any one of embodiments 468 to 489, wherein the one or more cellular features include an oligomeric state of the at least one extracellular molecule.

Embodiment 491 comprises the method of any one of embodiments 468 to 490, wherein the one or more cellular features include a heteromeric state of the at least one extracellular molecule.

Embodiment 492 comprises the method of any one of embodiments 468 to 491, wherein the one or more cellular features include one or more nearest neighbors to the at least one extracellular molecule.

Embodiment 493 comprises the method of embodiment 492, wherein the one or more nearest neighbors are selected from proteins, extracellular receptors, antigens, carbohydrates, and lipids.

Embodiment 494 comprises the method of any one of embodiments 479 to 493, wherein the one or more cellular features include a distance between separate molecules of the single extracellular molecule.

Embodiment 495 comprises the method of any one of embodiments 480 to 494, wherein the one or more cellular features include a pairwise distance between extracellular molecules of the extracellular molecule pair.

Embodiment 496 comprises the method of any one of embodiments 481 to 495, wherein the one or more cellular features include a pairwise distance between extracellular molecules of the extracellular molecule triplet, the extracellular molecule quadruplet, the extracellular molecule quintuplet, or other higher order extracellular molecule cluster.

Embodiment 497 comprises the method of any one of embodiments 468 to 496, wherein the one or more cellular features comprise lateral, anterior, or ventral movement of the at least one extracellular molecule.

Embodiment 498 comprises the method of any one of embodiments 468 to 497, wherein the one or more cellular features include one or more cellular features on a same cell and/or between different cells.

Embodiment 499 comprises the method of any one of embodiments 468 to 498, wherein the one or more cellular features include intercellular interactions between the at least one extracellular molecule and one or more extracellular proteins on a separate cell.

Embodiment 500 comprises the method of any one of embodiments 468 to 498, wherein the one or more cellular features comprise an absence of intercellular interactions between the at least one extracellular molecule and one or more extracellular proteins on a separate cell.

Embodiment 501 comprises the method of embodiments 499 or 500, wherein the intercellular interactions include an oligomer or a cluster of two or more extracellular proteins on separate cells.

Embodiment 502 comprises the method of any one of embodiments 499 to 501, wherein the intercellular interactions are selected from tumor cell-tumor cell interactions, tumor cell-tumor microenvironment cell interactions, and tumor microenvironment cell-tumor microenvironment cell interactions.

Embodiment 503 comprises the method of embodiment 502, wherein the cell in the tumor microenvironment is selected from a B cell, a T cell, a Natural Killer (NK) cell, a myeloid cell, a fibroblast, and a pericyte.

Embodiment 504 comprises the method of embodiment 503, wherein the cell in the tumor microenvironment is selected from a B cell, a T cell, a Natural Killer (NK) cell, a myeloid cell, a fibroblast, and a pericyte.

Embodiment 505 comprises the method of any one of embodiments 468 to 504, wherein the one or more cellular features predict the presence of a cancer cell or a cell in the tumor microenvironment.

Embodiment 506 comprises the method of any one of embodiments 468 to 504, wherein the one or more cellular features predict the presence of a normal cell.

Embodiment 507 comprises the method of any one of embodiments 468 to 506, wherein the one or more cellular features indicate a signaling event.

Embodiment 508 comprises the method of any one of embodiments 468 to 507, wherein the one or more cellular features define a molecular signature, cell signature or tissue sample signature.

Embodiment 509 comprises the method of any one of embodiments 468 to 508, wherein the one or more cellular features comprises an image of the at least one extracellular molecule.

Embodiment 510 comprises the method of any one of embodiments 468 to 509, wherein the one or more cellular features comprises two- or three-dimensional coordinates of the at least one extracellular molecule.

Embodiment 511 comprises the method of any one of embodiments 468 to 510, wherein the one or more cellular features comprises intensities for the at least one extracellular molecule.

Embodiment 512 comprises the method of any one of embodiments 468 to 511, wherein the one or more cellular features comprises all or part of a molecular fingerprint, molecular signature or map of the positions for the at least one extracellular molecule.

Embodiment 513 comprises the method of any one of embodiments 468 to 512, wherein the one or more cellular features comprises all or part of a schematic or topical representation of an extracellular molecule on a cell or tissue.

Embodiment 514 comprises the method of any one of embodiments 468 to 513, wherein the one or more cellular features comprises proximity between two or more extracellular molecules.

Embodiment 515 comprises the method of embodiment 514, wherein the two or more extracellular molecules are of the same extracellular molecule or different extracellular molecules.

Embodiment 516 comprises the method of any one of embodiments 468 to 515, wherein the one or more cellular features comprises interactions of at least two extracellular molecules.

Embodiment 517 comprises the method of any one of embodiments 468 to 516, wherein the one or more cellular features comprises a distance or distances between two or more extracellular molecules.

Embodiment 518 comprises the method of any one of embodiments 468 to 517, wherein the one or more cellular features comprises an individual extracellular molecule or pairs of extracellular molecules.

Embodiment 519 comprises the method of embodiment 518, wherein the individual extracellular molecule or pairs of extracellular molecules are adjacent or in contact with each other.

Embodiment 520 comprises the method of embodiment 518, wherein individual extracellular molecule or pairs of extracellular molecules are within a distance of about 1 nanometer of each other.

Embodiment 521 comprises the method of embodiment 518, wherein the individual extracellular molecule or pairs of extracellular molecules are with a distance of about 1 nanometer to about 100 nanometers of each other.

Embodiment 522 comprises the method of any one of embodiments 468 to 521, wherein the one or more cellular features comprises an individual extracellular molecule or pairs or triplets of extracellular molecules.

Embodiment 523 comprises the method of embodiment 522, wherein the individual extracellular molecule or pairs or triplets of extracellular molecules are adjacent or in contact with each other.

Embodiment 524 comprises the method of embodiment 522, wherein the individual extracellular molecule or pairs or triplets of extracellular molecules are within a distance of about 1 nanometer to about 100 nanometers of each other.

Embodiment 525 comprises the method of any one of embodiments 468 to 524, wherein the one or more cellular features comprises pairs, triplets, quadruplets, quintuplets, or higher order clusters of extracellular molecules.

Embodiment 526 comprises the method of embodiment 525, wherein the pairs, triplets, quadruplets, quintuplets, or higher order clusters of extracellular molecules are adjacent or in contact with each other.

Embodiment 527 comprises the method of embodiment 525, wherein the pairs, triplets, quadruplets, quintuplets, or higher order clusters of extracellular molecules comprise the same or different molecules.

Embodiment 528 comprises the method of any one of embodiments 468 to 527, wherein the one or more cellular features comprises a geometric relationship between two or more extracellular molecules.

Embodiment 529 comprises the method of any one embodiment 468 to 528, herein the one or more cellular features comprises movement of at least one extracellular molecule.

Embodiment 530 comprises the method of embodiment 529, wherein the movement is internalization or movement to an extracellular surface.

Embodiment 531 comprises the method of any one of embodiments 468 to 530, wherein the at least one cellular feature comprises internalization of at least one extracellular molecule.

Embodiment 532 comprises the method of any one of embodiments 468 to 531, wherein the one or more cellular features comprises a change in two- or three-dimensional coordinates of an extracellular molecule.

Embodiment 533 comprises the method of any one of embodiments 468 to 532, wherein the one or more cellular features comprises a change in clustering of an extracellular molecule.

Embodiment 534 comprises the method of any one of embodiments 468 to 533, wherein the one or more cellular features comprises a change in cluster formation of an extracellular molecule.

Embodiment 535 comprises the method of any one of embodiments 468 to 534, wherein the one or more cellular features comprises an oligomerization state of an extracellular molecule.

Embodiment 536 comprises the method of any one of embodiments 468 to 535, wherein the one or more cellular features comprises a clustering state of an extracellular molecule.

Embodiment 537 comprises the method of any one of embodiments 468 to 536, wherein the one or more cellular features comprises the names of proteins clustering with an extracellular molecule.

Embodiment 538 comprises the method of any one of embodiments 468 to 537, wherein the at least one extracellular molecule is an extracellular molecule listed in Table 1.

Embodiment 539 comprises the method of any one of embodiments 468 to 538, further comprising repeating the method for one or more additional cell or tissue samples comprising disease-state cells or non-disease state cells; and storing the one or more cellular features of each of the cell or tissue samples in a database to provide a database of cellular features of disease-state cells and non-disease state cells.

Embodiment 540 comprises the method of any one of embodiments 468 to 539, further comprising correlating a localized affinity (K_{d}) for binding of the antibody to the one or more cellular features of the cell or tissue sample stored in the database, and storing the correlation between the K_{d} and the one or more cellular features of the cell or tissue sample in the database.

Embodiment 541 comprises the method of embodiment 540, further comprising determining the localized K_{d} by single molecule tracking.

Embodiment 542 comprises the method of embodiments 540 or 541, further comprising predicting a localized K_{d} for binding of the antibody to a second cell or tissue sample based on a similarity between the one or more cellular features of the cell or tissue sample and one or more cellular features of the second cell or tissue sample.

Embodiment 543 comprises the method of any one of embodiments 468 to 542, wherein the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc.

Embodiment 544 comprises the method of any one of embodiments 468 to 543, wherein the antibody is a bispecific antibody.

Embodiment 545 comprises the method of any one of embodiments 468 to 543, wherein the antibody is a multispecific antibody.

Embodiment 546 comprises the method of any one of embodiments 468 to 545, wherein the antibody comprises at least one, at least two, at least three, at least four, at least five, or at least six VHH domains or more.

Embodiment 547 comprises the method of any one of embodiments 468 to 546, wherein the antibody comprises at least one, at least two, at least three, at least four, at least five, or at least six VL domains or more.

Embodiment 548 comprises the method of any one of embodiments 468 to 547, wherein the antibody comprises at least one, at least two, at least three, at least four, at least five, or at least six VH domains or more.

Embodiment 549 comprises the method of any one of embodiments 468 to 548, wherein the antibody is an immune cell engager.

Embodiment 550 comprises the method of any one of embodiments 468 to 548, wherein the antibody comprises an effector domain (e.g., CD3 or CD16A).

Embodiment 551 comprises the method of any one of embodiments 468 to 548, wherein the antibody-based therapy is on a CAR-T cell or myeloid cell or NK cell or other cell-based therapy.

Embodiment 552 comprises the method of any one of embodiments 468 to 551, wherein the antibody is in a vaccine.

Embodiment 553 comprises the method of any one of embodiments 468 to 552, wherein the antibody is a translated nucleic acid strand or strands.

Embodiment 554 comprises the method of any one of embodiments 468 to 553, wherein the antibody comprises a single chain polypeptide.

Embodiment 555 comprises the method of any one of embodiments 468 to 554, wherein the antibody comprises a homodimer.

Embodiment 556 comprises the method of any one of embodiments 468 to 555, wherein the antibody comprises a heterodimer.

Embodiment 557 comprises the method of any one of embodiments 468 to 556, wherein the super-resolution microscopy comprises stimulated emission depletion (STED) microscopy.

Embodiment 558 comprises the method of any one of embodiments 468 to 556, wherein the super-resolution microscopy comprises ground state depletion (GSD) microscopy.

Embodiment 559 comprises the method of any one embodiments 468 to 556, wherein the super-resolution microscopy comprises stochastic optical reconstruction microscopy (STORM) microscopy.

Embodiment 560 comprises the method of any one of embodiments 468 to 556, wherein the super-resolution microscopy comprises MINFLUX microscopy.

Embodiment 561 comprises the method of any one of embodiments 468 to 560, wherein the cell or tissue sample comprises a fixed cell.

Embodiment 562 comprises the method of any one of embodiments 468 to 560, wherein the cell or tissue sample comprises a live cell.

Embodiment 563 comprises the method of any one of embodiments 468 to 562, wherein the cell or tissue sample comprises a mammalian cell.

Embodiment 564 comprises the method of any one of embodiments 468 to 563, wherein imaging the cell or tissue sample comprises imaging the cell on a tissue microarray, coverslip, flow cell, chip, or microfluidics chamber.

Embodiment 565 comprises the method of embodiment 564, wherein the tissue microarray, coverslip, flow cell, chip, or microfluidics chamber comprises frozen tissue.

Embodiment 566 comprises the method of embodiment 564, wherein the tissue microarray, coverslip, flow cell, chip, or microfluidics chamber comprises live tissue.

Embodiment 567 comprises the method of embodiment 565, wherein the tissue microarray, coverslip, flow cell, chip, or microfluidics chamber comprises fixed tissue.

Embodiment 568 comprises the method of any one of embodiments 468 to 567, wherein the imaging is performed at a resolution of at least about 100 nanometers.

Embodiment 569 comprises the method of any one of embodiments 468 to 567, wherein the imaging is performed at a resolution of at least about 50 nanometers.

Embodiment 570 comprises the method of any one of embodiments 468 to 567, wherein the imaging is performed at a resolution of at least about 40 nanometers.

Embodiment 571 comprises the method of any one of embodiments 468 to 567, wherein the imaging is performed at a resolution of at least about 30 nanometers.

Embodiment 572 comprises the method of any one of embodiments 468 to 567, wherein the imaging is performed at a resolution of at least about 20 nanometers.

Embodiment 573 comprises the method of any one of embodiments 468 to 567, wherein the imaging is performed at a resolution of at least about 10 nanometers.

Embodiment 574 comprises the method of any one of embodiments 468 to 567, wherein the imaging is performed at a resolution of at least about 5 nanometers.

Embodiment 575 comprises the method of any one of embodiments 468 to 567, wherein the imaging is performed at a resolution of at least about 2 nanometers.

Embodiment 576 comprises the method of any one of embodiments 468 to 567, wherein the imaging is performed at a resolution of at least about 1 nanometers.

Embodiment 577 comprises the method of any one of embodiments 468 to 567, wherein the imaging is performed at a single molecule resolution.

Embodiment 578 comprises the method of any one of embodiments 468 to 567, wherein the imaging is performed at a single fluorophore resolution.

Embodiment 579 comprises the method of any one of embodiments 468 to 578, wherein the imaging is performed in high throughput format.

Embodiment 580 comprises the method of embodiment 579, wherein the high throughput format comprises use of at least one 96 well plate.

Embodiment 581 comprises the method of embodiment 579, wherein the high throughput format comprises use of at least one 192 well plate.

Embodiment 582 comprises the method of embodiment 579, wherein the high throughput format comprises use of at least one 384 well plate.

Embodiment 583 comprises the method of embodiment 579, wherein the high throughput format comprises use of at least one 1536 well plate.

Embodiment 584 comprises the method of embodiment 579, wherein the high throughput format comprises use of slides, coverslips, or flow cell containing multiple channels.

Embodiment 585 comprises the method of any one of embodiments 468 to 584, wherein the at least one extracellular molecule is a membrane-bound protein or a ligand bound to a membrane.

Embodiment 586 comprises the method of any one of embodiments 468 to 585, wherein the at least one extracellular molecule is selected from a protein ligand, a sugar, a lipid, a ligand, an extracellular receptor, a membrane-bound protein, a soluble protein, and a structural protein.

Embodiment 587 comprises the method of any one of embodiments 468 to 586, further comprising determining which of the one or more cellular features are consistent on a cancer cell line for one or more indications.

Embodiment 588 comprises the method of any one of embodiments 468 to 587, further comprising determining which of the one or more cellular features are unique to a cancer cell line for one or more indications.

Embodiment 589 comprises the method of any one of embodiments 468 to 588, further comprising determining which of the one or more cellular features are consistent on non-disease state cells.

Embodiment 590 comprises the method of any one of embodiments 468 to 589, further comprising determining which of the one or more cellular features differ on cancer cells and non-disease state cells.

Embodiment 591 comprises the method of any one of embodiments 468 to 590, further comprising determining which of the one or more cellular features predict clinical toxicity.

Embodiment 592 comprises the method of any one of embodiments 468 to 591, further comprising detecting a binding mode of the antibody to the at least one extracellular molecule based on the image.

### EXAMPLES

### Example 1: Antibody Sequences

Representative VHH antibody sequences of the present disclosure are listed in Table 2.

TABLE 2
VHH Antibody Sequences

| | | SEQ |
|---|---|---|
| | | ID |
| Description | Sequence | NO. |
| TROP2_1 | QVQLLESGGGLVQPGGSLRLSCAASGRTFSSQSMGWFRQAP | 1 |
| (anti-TROP2 VHH | GKEREFVSAISWTGANPTYADSVKGRFTISRDNSKNTLYLQ | |
| binder 1) | MNSLRAEDTAVYYCAADTSGGSYYYERATAETSYDYWGQ | |
| | GTLVTVSS | |
| TROP2_2 | QVQLLESGGGLVQPGGSLRLSCAASGFTFDYYAIGWFRQAP | 2 |
| (anti-TROP2 VHH | GKEREGVSCISSSHGSTYYADSVKGRFTISRDNSKNTVYLQ | |
| binder 2) | MNSLRAEDTAVYYCATAGDGGDYHCSGLVDYGMDYWGK | |
| | GTLVTVSS | |
| TROP2_3 | QVQLLESGGGLVQPGGSLRLSCLASGRTVGRTAMGWFRQP | 3 |
| (anti-TROP2 VHH | PGKEREFVATISWAGGTTYYADFVKGRFTISRDNSKNTLYL | |
| binder 3) | QMNSLRAEDTAVYYCAASEPYSDYDPSGMVYWGKGTLVT | |
| | VSS | |
| TROP2_4 | QVQLLESGGGLVQPGGSLRLSCAASGRTFGRAAMGWFRQP | 4 |
| (anti-TROP2 VHH | PGKEREFAATISWSGSNTYYADFVKGRFTISRDNSKNTVYL | |
| binder 4) | QMNSLRAEDTAVYYCAASEPYSDYDPSGMVYWGKGTLVT | |
| | VSS | |
| HER2_1 | EVQLVESGGSLVQPGGSLRLSCAASGFTFDDYAMSWVRQV | 5 |
| (anti-HER2 VHH | PGKGLEWVSSINWSGTHTDYADSVKGRFTISRNNANNTLYL | |
| binder 1) | QMNSLKSEDTAVYYCAKNWRDAGTTWFEKSGSAGQGTQV | |
| | TVSS | |
| HER_2 | EVQLVESGGGLVQAGGSLRLSCAASGITFSINTMGWYRQAP | 6 |
| (anti-HER2 VHH | GKQRELVALISSIGDTYYADSVKGRFTISRDNAKNTVYLQM | |
| binder 2) | NSLKPEDTAVYYCKRFRTAAQGTDYWGQGTQVTVSS | |
| HER2_3 | KVQLVESGGGLVQPGGSLRLSCAASGSIFGFNDMAWYRQA | 7 |
| (anti-HER2 VHH | PGKQRELVALISRVGVTSSADSVKGRFTISRVNAKDTVYLQ | |
| binder 3) | MNSLKPEDTAVYYCYMDQRLDGSTLAYWGQGTQVTVSS | |
| EPCAM_1 | QVQLVESGGGLVQPGGSLTLSCAASGTGSIFSINLMGWYRQ | 8 |
| (anti-EPCAM VHH | APGKQRELVARITSGDSTVYADSVKGRFTISRDNSKNTLYL | |
| binder 1) | QMNSLRPEDTAVYYCNLLLRSSPGATTPYWGQGTLVTVSS | |
| EPCAM_2 | QVQLVESGGGLVQPGGSLTLSCVISGSFSALWAMRWYRQA | 9 |
| (anti-EPCAM VHH | PGQQRELVASSRGGTTSYADSVKGRFTISRDNSKNTLYLQM | |
| binder 2) | NSLRPEDTAVYYCNAIDGHLAYWGQGTLVTVSS | |
| EPCAM_3 | QVQLLESGGGLVQPGGSLRLSCAASGRTFSDYDMGWFRQG | 10 |
| (anti-EPCAM VHH | PGKEREFVAAISWSGGHTNYADSVKGRFTISRDNSKNTLYL | |
| binder 3) | QMNSLRAEDTAVYYCAADLRFTGGDTTTPETYDYWGQGT | |
| | LVTVSS | |
| EPCAM_4 | QVQLVESGGGLVQPGRSLRLSCAASGRTLDNYDMGWFRQG | 11 |
| (anti-EPCAM VHH | PGKEREFVAAISWSGGSTDYAYSVTGRFTISRDNAKNSLYL | |
| binder 4) | QMNSLRAEDTALYYCAADLRFTGGDTMTPETYDYWGQGT | |
| | LVTVSS | |
| BABA 1 | QVQLQESGGGLVQPGGSLRLSCAASGSIYSLIAMGWYRQAP | 12 |
| (anti-H. pylori | GKEHELVATISSGSTTYYADSVKGRFTISRDNAKNTLYLQM | |
| BabA VHH) | NSLKPEDTAMYYCAAYSDRLTDCSNCEADYWGQGTQVTV | |
| | SH | |

VHH antibodies were fluorescently labeled with KK114 via a linker sequence. KK114 has excitation wavelengths between 630 to 650 nanometers and depletion wavelengths around 750 to 800 nm).

### Example 2: STED Imaging of Cells Stained with Fluorescently Labeled Antibodies

STED imaging experiments with cells stained with antibodies was carried out using an Abberior STED instrument (model STEDYCON). For STED and Confocal imaging, a 640 nanometer (nm) excitation laser was used and its attenuation was set to 0.9. For STED imaging, a 775 nm laser was used for stimulated depletion. Images were acquired at 16 bits per pixel via line accumulation with an avalanche photodiode (APD) detector, a 40 mm pinhole size, an emission detection wavelength set to 675 nm, and an objective with 60× magnification. On the inverted microscope, the focal plane was aligned with the solvent facing membrane of the adherent cell by adjusting the Z-axis. Cells were identified by course scan confocal imaging and selected and queued as regions of interest. Each region of interest was imaged in confocal and in STED mode, resulting in two images.

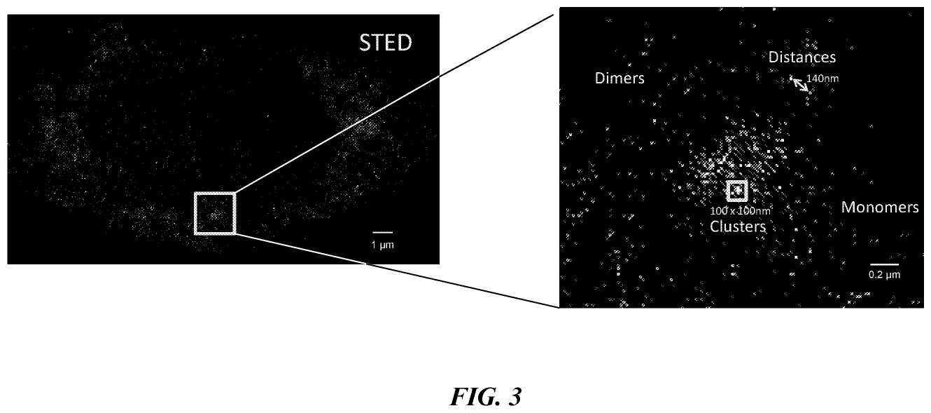

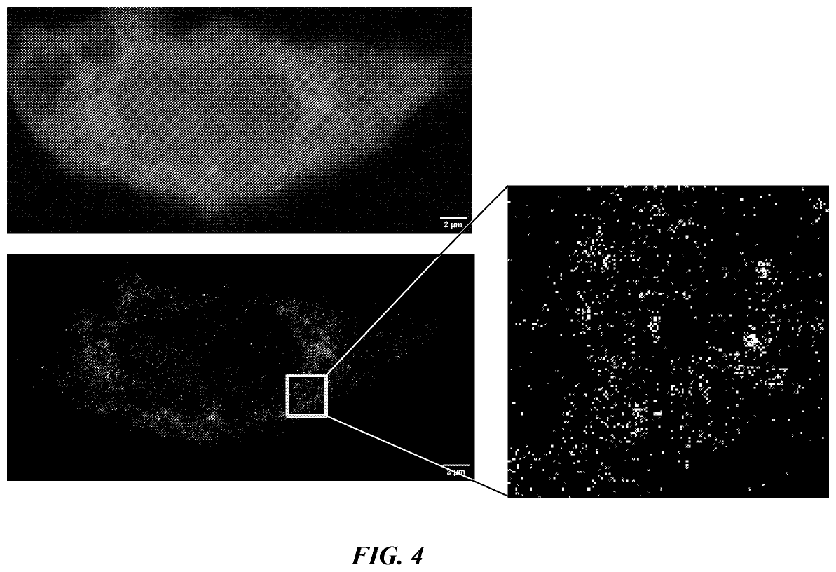

FIG. 3 shows a STED image of A549 cells stained with labeled anti-TROP2 VHH binder 1 (TROP2_1), wherein the image is 34.68×17.04 micrometers (μm) (1734×852 pixels) with a resolution of 20 nanometers (nm)/pixel. FIG. 4 shows a STED image of A549 cells stained with labeled TROP2_1, wherein the image is 34.68×17.04 μm (1734×852 pixels) with a resolution of 20 nm/pixel. The images were acquired with emission at 660 nm, excitation laser at 640 nm set to 20% power, STED depletion laser at 775 nm set to 5% power. 50 acquisitions were accumulated. Pinhole was set to 1.0 AU and a dwell time of 5 microseconds (usec) was used.

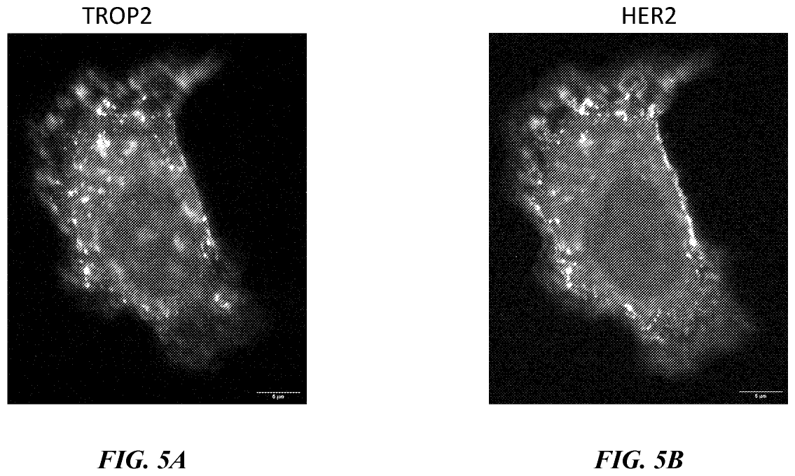

FIGS. 5A-5B show SKBR3 cells stained with labeled TROP2_1 (FIG. 5A) and labeled anti-HER2 VHH binder 3 (HER2_3) (FIG. 5B), wherein the image is 34.60×43.06 μm (1730×2153 pixels) with a resolution of 20 nm/pixel. Pinhole was set to 1.0 AU with a dwell time of 5 usec. The TROP2 image was acquired with emission at 660 nm, excitation laser at 640 nm set to 20% power, STED depletion laser at 775 nm set to 5% power, 25 acquisitions were accumulated. The HER2 image was acquired with emission at 616 nm, excitation laser at 561 nm set to 20% power, STED depletion laser at 775 nm set to 5% power, 25 acquisitions were accumulated.

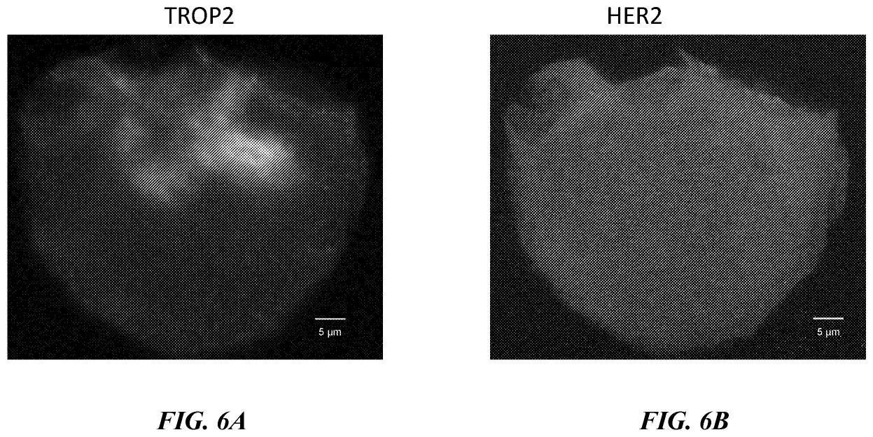

FIGS. 6A-6B show MCF7 cells stained with labeled TROP2_1 (FIG. 6A) and labeled HER2_3 (FIG. 6B), wherein the images are 63.86×58.30 μm (3193×2915 pixels) with a resolution of 20 nm/pixel. Pinhole was set to 1.0 AU with a dwell time of 5 usec. The TROP2 image was acquired with emission at 660 nm, excitation laser at 640 nm set to 20% power, STED depletion laser at 775 nm set to 5% power, 25 acquisitions were accumulated. The HER2 image was acquired with emission at 616 nm, excitation laser at 561 nm set to 20% power, STED depletion laser at 775 nm set to 10% power, 25 acquisitions were accumulated.

### Example 3: Exemplary Mixing Experiment to Increase Resolution

The antibody (e.g., VHH) will be labeled to a particular target with red and orange fluorophores (e.g., K114 and Abberior STAR ORANGE) in two separate coupling reactions. The red and orange labeled antibodies will be mixed at a 1:1 ratio and used to stain cells/tissues. Cells/tissues will be subsequently fixed and the coverslip will be mounted on a microscope slide. Super-resolution microscopy images will be recorded using two colors, i.e., creating a red image and an orange image and the images will be overlaid. In the overlayed SRM image, a monomer will be either red or orange and be of relative intensity 1, and a dimer will be either red of intensity 2, orange of intensity 2, or show a red and orange signal of intensity 1 each. The frequency of observed dimers will be 1:1:2 for red:orange:red+orange. Trimer will have the following distribution of colors:red (intensity 3)| orange (intensity 3)| red (intensity 2)+orange (intensity 1)| red (intensity 1)+orange (intensity 2) with a frequency distribution as 1:1:3:3 for the 4 different color signatures. Tetramers may be more complex. Depending on signal intensities and distribution of signals, one can fit the observed peaks to either monomers, dimers, tetramers, or higher multimer.

### Example 4: Characterization of Normal and Cancer Cell Lines

Differences and similarities between normal and cancer cell lines will be determined. It will be confirmed whether protein configuration, protein-protein interactions, molecular complexes, and molecular signatures directly translate to therapeutic strategies (e.g., target discovery, drug design, etc.) in a model system (cell lines). Further, it will be determined whether these features can predict clinical toxicities.

30 VHHs will be multiplexed to cancer proteins on fixed cancer and normal cell lines. Four different cancer indications will be used including lung, colorectal, breast, and prostate cancer cells, and eight cancer cell lines per indication will be used. As many normal cell lines as possible will be used including normal colon, lung, breast, and prostate tissue, as well as known sites of toxicity for antibody drugs in cancer clinical trials (e.g., skin, eye, heart, gastrointestinal tissue). Controls will include marketed/development-stage antibodies, different protein target binders, non-binders, staining concentration and procedure, sample washing procedure, amount of fixative and fixing procedure, and sample integrity after multiplexing. Considerations include nanobody selection, nanobody interference in multiplexing, and oligomeric protein state.

### Example 5: Drug Design and Lead Development Candidate Selection (Cells)

An understanding of protein structure in its cellular context may guide drug design and lead development candidate selection. It will be determined whether different antibody formats have distinct binding modes on the surface of cancer and normal cells. Further, it will be determined whether known drugs bind to the same features on cancer cells and normal cells where toxicity is observed in cancer patients. It will be determined if certain bispecific antibody formats can distinguish cancer and normal cells.

Different concentrations of three antibody drugs, their corresponding target VHHs, and lead candidates will be titrated on cancer and normal cell lines. Four different cancer indications will be used including lung, colorectal, breast, and prostate cancers. Eight cancer cell lines will be used per indication. As many normal cell lines as possible will be used including normal colon, lung, breast, and prostate tissue, as well as known sites of toxicity for antibody drugs in cancer clinical trials (e.g., skin, eye, heart, stomach/colorectal). Considerations include antibody saturation vs. detecting antibody binding modes, and the role of ligand binding for some protein targets.

### Example 6: Case Study-Avidity/Drug Selectivity in Acute Myeloid Leukemia (AML) Clinical Trials

AMV564 is bivalent, affinity-matured version of the monovalent AMG330. Both T cell engagers target CD33, a myeloid cell lineage marker expressed on leukemic blasts, myeloid-derived suppressor cells (MDSCs), and normal myeloid cells (monocytes, macrophages, neutrophils). AMV564 was selected for clinical trials based on its ability to potentially kill HL60 and KGla cells which have different levels of CD33 expression. Some lead candidates in the AMV564 preclinical program had weak killing activity against KGla cells. AMV564, but not AMG330, is highly selective for leukemic blasts and MDSCs in AML clinical trials.

The HL60 and KGla cancer cell lines and normal myeloid cells/cell lines will be characterized. Different concentrations of AMV564 and related antibodies (i.e., which don't bind to KGla cells), AMG330, CD33 VHH, and certain CD33 targeted antibodies will be titrated on the cancer and normal cell lines. It will be determined whether differences in the way the CD33 target is presented on HL60, KGla, MDSCs versus normal myeloid cells predict drug selectivity in the clinic of AMV564 versus AMG330. Considerations include accessing key myeloid cell types (e.g., neutrophils), and correlation with clinical safety data.

### Example 7: Characterization of Normal and Cancer Tissue and/or Disseminated Cells

Studies similar to Example 4 will be carried out except tissue samples or disseminated cells from tumor biopsies, laser microdissection, or other tissue model will be used. Normal and cancer tissue and/or disseminated cells will be characterized. Differences and similarities will be determined. It will be confirmed that protein configuration, protein-protein interactions, molecular complexes, and molecular signatures directly translate to therapeutic strategies (e.g., target discovery, drug design) in tissue and/or disseminated cells. Further, it will be determined whether these features can predict clinical toxicities. Considerations include profiling tissue (i.e., single cell, single molecule), tissue heterogeneity, impact of tumor microenvironment, prior drug treatment, identification of cell types in the tumor microenvironment, analytical tools (that capture location, cell type, adjacent cell types, molecules interacting on different cells, and have an ability to distinguish cell membranes), impact of ligand binding to protein, and analysis to include comparison of cell and tissue data.

### Example 8: Drug Design and Lead Development Candidate Selection (Tissue Samples or Disseminated Cells)

Studies similar to Example 5 will be carried out except that tissue samples or disseminated cells from tumor biopsies, laser microdissection, or other tissue model samples will be used. It will be determined whether different antibody formats have distinct binding modes on the surface of cancer and normal tissue or disseminated cells. Further, it will be determined if known drugs bind to the same features normal or disseminated cells where toxicity is observed in cancer patients. It will be determined if certain bispecific antibody formats can distinguish cancer and normal tissue or disseminated cells. Considerations include tissue heterogeneity, impact of tumor microenvironment, prior drug treatment, identifying cell types in the tumor microenvironment, analytical tools (that capture location, cell type, adjacent cell types, molecules interacting on different cells, and that have an ability to distinguish cell membranes), impact of ligand binding to protein, and analysis to include comparison of cell and tissue data.

Additional studies similar to Examples 4-8 may incorporate live cell or live tissue. Furthermore, additional investigations may involve single molecule tracking including internalization and cell surface motion, as well as binding studies with ligands on cell lines or tissue samples.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method of generating a database comprising cellular features from a disease-state cell and non-disease state cell comprising imaging a sample that has been contacted by an antibody using a super-resolution microscope and generating a database comprising at least one cellular feature of a cell in the sample and determining if the at least one cellular feature is indicative of the disease-state cell or the non-disease-state cell.

2. The method of claim 1, wherein the at least one cellular feature comprises an image of an extracellular molecule.

3. The method of claim 1, wherein the at least one cellular feature comprises two- or three-dimensional coordinates of an extracellular molecule.

4. The method of claim 1, wherein the at least one cellular feature comprises intensities for an extracellular molecule.

5. The method of claim 1, wherein the at least one cellular feature comprises all or part of a molecular fingerprint, molecular signature, or map of the positions for an extracellular molecule.

6. The method of claim 1, wherein the at least one cellular feature comprises all or part of a schematic or topical representation of an extracellular molecule on a cell or tissue.

7. The method of claim 1, wherein the at least one cellular feature comprises proximity between two or more extracellular molecules.

8. The method of claim 7, wherein the two or more extracellular molecules are of the same target or different targets.

9. The method of claim 1, wherein the at least one cellular feature comprises interactions of at least two extracellular molecules.

10. The method of claim 1, wherein the at least one cellular feature comprises a distance or distances between two or more extracellular molecules.

11. The method of claim 1, wherein the at least one cellular feature comprises an individual extracellular molecule or pairs of extracellular molecules.

12. The method of claim 11, wherein the individual extracellular molecule or pairs of extracellular molecules are adjacent or in contact with each other.

13. The method of claim 11, wherein the individual extracellular molecule or pairs of extracellular molecules are within a distance of about 1 nanometer of each other.

14. The method of claim 11, wherein the individual extracellular molecule or pairs of extracellular molecules are with a distance of about 1 nanometer to about 100 nanometers of each other.

15. The method of claim 1, wherein the at least one cellular feature comprises an individual extracellular molecule, pairs of extracellular molecules, or triplets of extracellular molecules.

16. The method of claim 15, wherein the individual extracellular molecule, pairs of extracellular molecules, or triplets of extracellular molecules are adjacent or in contact with each other.

17. The method of claim 15, wherein the individual extracellular molecule, pairs of extracellular molecules, or triplets of extracellular molecules are within a distance of about 1 nanometer to about 100 nanometers of each other.

18. The method of claim 1, wherein the at least one cellular feature comprises pairs, triplets, quadruplets, quintuplets, or higher order clusters of extracellular molecules.

19. The method of claim 18, wherein the pairs, triplets, quadruplets, quintuplets, or higher order clusters are adjacent or in contact with each other.

20. The method of claim 18, wherein the pairs, triplets, quadruplets, quintuplets, or higher order clusters comprise the same or different extracellular molecules.

21. The method of claim 1, wherein the at least one cellular feature comprises a geometric relationship between two or more extracellular molecules.

22. The method of claim 1, wherein the at least one cellular feature comprises movement of at least one extracellular molecule.

23. The method of claim 22, wherein the movement is internalization or movement to an extracellular surface.

24. The method of claim 1, wherein the at least one cellular feature comprises internalization of at least one extracellular molecule.

25. The method of claim 1, wherein the at least one cellular feature comprises a change in two- or three-dimensional coordinates of an extracellular molecule.

26. The method of claim 1, wherein the at least one cellular feature comprises a change in clustering of an extracellular molecule.

27. The method of claim 1, wherein the at least one cellular feature comprises a change in cluster formation of an extracellular molecule.

28. The method of claim 1, wherein the at least one cellular feature comprises an oligomerization state of an extracellular molecule.

29. The method of claim 1, wherein the at least one cellular feature comprises a clustering state of an extracellular molecule.

30. The method of claim 1, wherein the at least one cellular feature comprises the names of proteins clustering with an extracellular molecule.

31. The method of claim 2, wherein the extracellular molecule is an extracellular molecule listed in Table 1.

32. The method of claim 1, wherein the at least one cellular feature is present on a cell in tissue.

33. The method of claim 1, wherein the at least one cellular feature is present on a cell in cancer or on normal tissue.

34. The method of claim 1, wherein the at least one cellular feature is present on a normal cell, on a cancer cell or both normal and cancer cells.

35. The method of claim 1, wherein the at least one cellular feature is present on a normal cell, a cancer cell or a cell in the tumor microenvironment.

36. The method of claim 1, wherein the at least one cellular feature is present on a cell in the tumor microenvironment.

37. The method of claim 36, wherein the cell in the tumor microenvironment is selected from a B cell, a T cell, a Natural Killer (NK) cell, a myeloid cell, a fibroblast, and a pericyte.

38. The method of claim 36, wherein the cell in the tumor microenvironment is selected from a stromal cell, an epithelial cell or an adipocyte.

39. The method of claim 1, wherein the at least one cellular feature predicts the presence of a cancer cell or a cell in the tumor microenvironment.

40. The method of claim 1, wherein the at least one cellular feature predicts the presence of a normal cell.

41. The method of claim 1, wherein the at least one cellular feature is present on the same cell in tissue.

42. The method of claim 1, wherein the at least one cellular feature is present on at least two different cells in tissue.

43. The method of claim 42, wherein the at least one cellular feature is within a distance or between cell distances of the at least two different cells.

44. The method of claim 1, wherein the at least one cellular feature is present on a tissue structure in a normal or a diseased state.

45. The method of claim 44, wherein the diseased state comprises a cancer diseased state, an immunological diseased state, a neurological diseased state, an antiviral diseased state, a cardiovascular diseased state, or an autoimmune diseased state.

46. The method of claim 44, wherein the tissue structure comprises epithelium, a duct, or a blood vessel.

47. The method of claim 1, wherein the at least one cellular feature comprises a change in expression of an extracellular molecule.

48. The method of claim 1, wherein the at least one cellular feature comprises a change in a glycosylation pattern of an extracellular molecule.

49. The method of claim 1, wherein the at least one cellular feature comprises a change in enzymatic activity of an extracellular molecule.

50. The method of claim 1, wherein the at least one cellular feature comprises a change in intracellular or intercellular communication.

51. The method of claim 1, wherein the at least one cellular feature comprises a change in intracellular or intercellular signaling or communication.

52. The method of claim 1, wherein the at least one cellular feature comprises a change in cell adhesion, mechanics or movement.

53. The method of claim 1, wherein the at least one cellular feature comprises tissue localization of an extracellular molecule.

54. The method of claim 53, wherein the tissue localization comprises a diseased tissue or tumor microenvironment.

55. The method of claim 53, wherein the tissue localization comprises a cancer structure or tumor microenvironment.

56. The method of claim 53, wherein the extracellular molecule internalizes when contacted by the antibody.

57. The method of claim 1, wherein different samples are assessed for common cellular features.

58. The method of claim 1, wherein different samples are assessed for different cellular features.

59. The method of claim 1, further comprising characterizing the at least one cellular feature based on an intensity of an imaging agent in the image.

60. The method of claim 1, wherein the at least one cellular feature is assessed after administration of at least one ligand.

61. The method of claim 1, wherein the at least one cellular feature is assessed after administration of at least one therapeutic or at least one therapeutic regimen.

62. The method of claim 1, wherein the super-resolution microscope comprises a deterministic super resolution microscope.

63. The method of claim 1, wherein the super-resolution microscope is a stimulated emission depletion (STED) microscope or a ground state depletion (GSD) microscope.

64. The method of claim 1, wherein the super-resolution microscope comprises a stochastic super-resolution microscope.

65. The method of claim 1, wherein the super-resolution microscope is a MINFLUX microscope.

66. The method of claim 1, wherein the super-resolution microscope is a stochastic optical reconstruction microscopy (STORM) microscope.

67. The method of claim 1, wherein the imaging comprises tracking the one ore more ligands, therapeutics, or cell surface receptors over a period of time.

68. The method of claim 1, wherein the cell comprises a fixed cell.

69. The method of claim 1, wherein the cell is fixed in tissue.

70. The method of claim 1, wherein the cell is in live tissue.

71. The method of claim 1, wherein the cell comprises a live cell.

72. The method of claim 1, wherein the cell comprises a mammalian cell.

73. The method of claim 1, wherein the disease state cells are derived from a tumor cell line.

74. The method of claim 1, wherein the normal state cells are derived from a normal cell line.

75. The method of claim 1, wherein the cell is imaged on a tissue microarray, coverslip, flow cell, chip, or microfluidics chamber.

76. The method of claim 75, wherein the tissue microarray, coverslip, flow cell, chip, or microfluidics chamber comprises frozen tissue.

77. The method of claim 75, wherein the tissue microarray, coverslip, flow cell, chip, or microfluidics chamber comprises live tissue.

78. The method of claim 75, wherein the tissue microarray, coverslip, flow cell, chip, or microfluidics chamber comprises fixed tissue.

79. The method of claim 75, wherein the tissue microarray coverslip, flow cell, chip, or microfluidics chamber is formed via a laser microdissection process.

80. The method of claim 1, wherein the antibody comprises an antibody selected from a single chain variable fragment (scFv), a heavy chain variable domain (VH), a light chain variable domain (VL), a variable domain (VHH) of camelid derived single domain antibody, a synthetically derived VHH, a single domain antibody created in a mouse, a shark antibody, an antigen binding fragment (Fab), a monoclonal antibody, a F(ab′) fragment, a F(ab′)2 fragment, single chain antibodies, diabodies, and a scFv-Fc.

81. The method of claim 80, wherein the antibody is a bispecific antibody.

82. The method of claim 80, wherein the antibody is a multispecific antibody.

83. The method of claim 80, wherein the antibody comprises at least one, at least two, at least three, at least four, at least five, or at least six VHH domains or more.

84. The method of claim 80, wherein the antibody comprises at least one, at least two, at least three, at least four, at least five, or at least six VL domains or more.

85. The method of claim 80, wherein the antibody comprises at least one, at least two, at least three, at least four, at least five, or at least six VH domains or more.

86. The method of claim 80, wherein the antibody is an immune cell engager.

87. The method of claim 80, wherein the antibody comprises an effector domain (e.g., CD3 or CD16A).

88. The method of claim 80, wherein the antibody-based therapy is on a CAR-T cell or myeloid cell or NK cell or other cell-based therapy.

89. The method of claim 80, wherein the antibody is in a vaccine.

90. The method of claim 80, wherein the antibody is a translated nucleic acid strand or strands.

91. The method of claim 80, wherein the antibody comprises a single chain polypeptide.

92. The method of claim 80, wherein the antibody comprises a homodimer.

93. The method of claim 80, wherein the antibody comprises a heterodimer.

94. The method of claim 1, wherein the database of imaged cellular features are imaged at a resolution of at least about 50 nanometers.

95. The method of claim 1, wherein the database of imaged cellular features are imaged at a resolution of at least about 40 nanometers.

96. The method of claim 1, wherein the database of imaged cellular features are imaged at a resolution of at least about 30 nanometers.

97. The method of claim 1, wherein the database of imaged cellular features are imaged at a resolution of at least about 20 nanometers.

98. The method of claim 1, wherein the database of imaged cellular features are imaged at a resolution of at least about 10 nanometers.

99. The method of claim 1, wherein the database of imaged cellular features are imaged at a resolution of at least about 9 nanometers.

100. The method of claim 1, wherein the database of imaged cellular features are imaged at a resolution of at least about 8 nanometers.

101. The method of claim 1, wherein the database of imaged cellular features are imaged at a resolution of at least about 7 nanometers.

102. The method of claim 1, wherein the database of imaged cellular features are imaged at a resolution of at least about 6 nanometers.

103. The method of claim 1, wherein the database of imaged cellular features are imaged at a resolution of at least about 5 nanometers.

104. The method of claim 1, wherein the database of imaged cellular features are imaged at a resolution of at least about 4 nanometers.

105. The method of claim 1, wherein the database of imaged cellular features are imaged at a resolution of at least about 3 nanometers.

106. The method of claim 1, wherein the database of imaged cellular features are imaged at a resolution of at least about 2 nanometers.

107. The method of claim 1, wherein the database of imaged cellular features are imaged at a resolution of at least about 1 nanometers.

108. The method of claim 1, wherein the database of imaged cellular features are imaged at a single molecule resolution.

109. The method of claim 1, wherein the database of imaged cellular features are imaged at a single fluorophore resolution.

110. The method of claim 1, wherein the database of imaged cellular features are imaged in a high throughput format.

111. The method of claim 110, wherein the high throughput format comprises use of at least one 96 well plate.

112. The method of claim 110, wherein the high throughput format comprises use of at least one 192 well plate.

113. The method of claim 110, wherein the high throughput format comprises use of at least one 384 well plate.

114. The method of claim 110, wherein the high throughput format comprises use of at least one 1536 well plate.

115. The method of claim 110, wherein the high throughput format comprises the use of slides, coverslips, or flow cells containing multiple channels.

116. The method of claim 1, wherein multiple cellular features are imaged at substantially the same time.

117. The method of claim 1, wherein multiple cellular features are imaged sequentially.

118. The method of claim 1, wherein multiple cellular features are images at substantially the same time and others are imaged sequentially.

119. The method of claim 118, wherein the multiple cellular features are on surfaces of multiple cells.

120. The method of claim 1, wherein imaging of a cellular feature is completed in less than about 5 minutes.

121. The method of claim 1, wherein imaging the database of imaged cellular features is done autonomously.

122. The method of claim 2, wherein the extracellular molecule is a membrane-bound protein, a ligand bound to a membrane, or a protein or ligand bound to a membrane protein.

123. The method of claim 2, wherein the extracellular molecule is selected from a protein ligand, a sugar, a lipid, a ligand, an extracellular receptor, a membrane-bound protein, a soluble protein, a structural protein, and a protein or ligand bound to a membrane protein.


SEQUENCE LISTING (12 sequences)

SEQ ID NO: 1 - protein, 129 aa, synthetic construct
QVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSS

SEQ ID NO: 2 - protein, 128 aa, synthetic construct
QVQLLESGGGLVQPGGSLRLSCAASGFTFDYYAIGWFRQAPGKEREGVSCISSSHGSTYYADSVKGRFTISRDNSKNTVYLQMNSLRAEDTAVYYCATAGDGGDYHCSGLVDYGMDYWGKGTLVTVSS

SEQ ID NO: 3 - protein, 123 aa, synthetic construct
QVQLLESGGGLVQPGGSLRLSCLASGRTVGRTAMGWFRQPPGKEREFVATISWAGGTTYYADFVKGRFTISRDNSKNTLYLQMNSLRAEDTAVYYCAASEPYSDYDPSGMVYWGKGTLVTVSS

SEQ ID NO: 4 - protein, 123 aa, synthetic construct
QVQLLESGGGLVQPGGSLRLSCAASGRTFGRAAMGWFRQPPGKEREFAATISWSGSNTYYADFVKGRFTISRDNSKNTVYLQMNSLRAEDTAVYYCAASEPYSDYDPSGMVYWGKGTLVTVSS

SEQ ID NO: 5 - protein, 124 aa, synthetic construct
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 6 - protein, 118 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 7 - protein, 119 aa, synthetic construct
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 8 - protein, 122 aa, synthetic construct
QVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSS

SEQ ID NO: 9 - protein, 114 aa, synthetic construct
QVQLVESGGGLVQPGGSLTLSCVISGSFSALWAMRWYRQAPGQQRELVASSRGGTTSYADSVKGRFTISRDNSKNTLYLQMNSLRPEDTAVYYCNAIDGHLAYWGQGTLVTVSS

SEQ ID NO: 10 - protein, 126 aa, synthetic construct
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSS

SEQ ID NO: 11 - protein, 126 aa, synthetic construct
QVQLVESGGGLVQPGRSLRLSCAASGRTLDNYDMGWFRQGPGKEREFVAAISWSGGSTDYAYSVTGRFTISRDNAKNSLYLQMNSLRAEDTALYYCAADLRFTGGDTMTPETYDYWGQGTLVTVSS

SEQ ID NO: 12 - protein, 123 aa, synthetic construct
QVQLQESGGGLVQPGGSLRLSCAASGSIYSLIAMGWYRQAPGKEHELVATISSGSTTYYADSVKGRFTISRDNAKNTLYLQMNSLKPEDTAMYYCAAYSDRLTDCSNCEADYWGQGTQVTVSH